(19) United States
(12) Patent Application Publication
(10) Pub. No.: US 2010/0215670 A1
(43) Pub. Date: Aug. 26, 2010

(54) Immunotoxin Fusions Comprising An Antibody Fragment and a Plant Toxin Linked by Protease Cleavable Linkers

(76) Inventors: Jeannick Cizeau, Winnipeg (CA); Glen MacDonald, Winnipeg (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/447,748

(22) PCT Filed: Oct. 30, 2007
(86) PCT No.: PCT/CA07/01927

§ 371 Date: Dec. 9, 2009

Related U.S. Application Data
(60) Provisional application No. 60/855,132, filed on Oct. 30, 2006.

(51) Int. Cl. A61K 39/395; C07K 16/00; C12N 15/13; C12N 15/63; C12N 1/00; G01N 33/574; A61P 35/00

(52) U.S. Cl. 424/178.1; 530/391.1; 530/391.7; 536/23.53; 435/320.1; 435/243; 435/7.23

(57) ABSTRACT

Novel conjugates are disclosed which comprise (a) a ligand that binds to a surface molecule on a target cell, such as a cancer cell; (b) an effector molecule that is to be delivered into the cell, such as a toxin; and (c) a linker sequence that couples the ligand and the effector molecule wherein the linker comprises at least one protease cleavage site corresponding to a protease found in the intracellular trafficking pathway of the effector molecule; wherein the cleavage of the linker by the protease uncouples the effector molecule from the ligand.

### FIELD OF THE INVENTION

The present invention relates to modified conjugates for optimal release of an effector having an improved cytoxicity comprising a protease sensitive linker selected according to the cellular trafficking pathway of the conjugate.

### BACKGROUND OF THE INVENTION

Targeted drug delivery has emerged as a potentially effective method for the treatment of disease. Specific effectors (i.e. chemotherapeutics, radionuclides, cytotoxins, cytokines) can be delivered to target cells by creating drug conjugates combining ligands that recognize particular cell surface proteins. Examples of these ligands are receptor recognized proteins or antibodies specific to epitopes on the cell surface. The effector can be directly conjugated to the ligand through recombinant means or indirectly through a carrier (e.g. liposome).

Antibodies specific to cell surface proteins can be used as a vehicle to carry and specifically deliver an effector such as a cytotoxin to cells. A recombinant antibody carrying a cytotoxin is referred to as an immunotoxin. Typically, peptide linkers are required between the effector and the antibody moiety to allow for the release of the effector from the antibody or ligand. Such peptide linkers are usually cleaved by intracellular proteases.

Proteins such as Ribosome Inactivating Proteins (RIPs) (review see Stirpe, 2000) are proteins that, upon internalisation, target the protein synthesis machinery of eukaryotic cells, thereby leading to cell death. Bouganin (Bolognesi et al., 1997) (den Hartog et al., 2002), is a type I RIP able to arrest protein synthesis by deadenylation of ribosomal RNA. (Monzingo and Robertus, 1992) (Ready et al., 1991). Bouganin lacks a cell binding subunit and therefore has a very low intrinsic cytotoxicity making it a suitable effector for the preparation of cytotoxic conjugates. Compared to other type I RIPs, bouganin has the lowest toxicity of any of the RIPs identified to date with the highest dose tested: 32 mg/kg in mice (den Hartog et al., 2002).

Upon internalization proteins traffic through intracellular organelles where they are degraded and/or modified. The trafficking pathway of certain protein cytotoxins such RIPs have been studied and defined to various extent. For example, saporin, diphtheria toxin anthrax toxin and PE upon endocytosis reach the endosome which latterly fuses to the lysosome. Others, after reaching the endosome, (ricin, shiga toxin and cholera toxin) traffic through the ER via the Golgi apparatus. Recently, PE has been shown to use the lysosome as well as the ER pathway to reach the cytosol (Smith et al., 2006). PE and cholera toxin use retrograde pathways to move from the Golgi apparatus to the ER. Once in the ER, cytotoxins still need to translocate from the ER compartment to the cytosol. Ricin and cholera toxin are known to use the ER Associated Degradation (ERAD) pathway to translocate from the ER to the cytosol (Deeks et al., 2002) (Tsai et al., 2001) (Rodighiero et al., 2002).

It is during the trafficking of a conjugate that an effector is separated from the ligand or moiety that allowed it to enter the cells. For synthetic conjugates, a cleavable linker may be used to allow the separation of the effector from the ligand. The effector is then expected to follow its usual trafficking pathway.

VB6-845-F-de-bouganin is an immunoconjugate consisting of a Fab fragment of an Ep-Cam binding antibody (VB6), a furin sensitive linker (F) and a deimmunized bouganin cytotoxin (de-bouganin). This conjugate binds to the Ep-CAM antigen found on the surface of carcinoma cells and is internalized. After internalization the immunoconjugate is cleaved by furin such that the de-bouganin effector is released from the Fab fragment and proceeds along its normal trafficking. The release of the de-bouganin from the Fab fragment is critical. See PCT/CA2005/000410 which is incorporated by reference.

The choice of linker for conjugate has been typically dictated by the type of enzymes that are either present in most cells, known to be upregulated in target cells, or selected from different regions of the cell (Fuchs et al., 2006) (Heisler et al., 2003) (Keller et al., 2001). Some proteases are ubiquitously expressed. Others are linked to particular cell types or areas within or around the cells. A furin-sensitive linker is commonly used because furin is a ubiquitous enzyme known to be present in both the endosome and the Golgi apparatus of most cells and is thus expected to cleave a conjugate in any cell it enters.

### SUMMARY OF THE INVENTION

The inventors have prepared modified conjugates, such as immunotoxins, that have improved cytotoxicity. The modified conjugates are prepared by inserting a protease sensitive linker comprising one or more protease cleavage or recognition sites that correspond to a protease or multiple proteases associated with or found in an intracellular trafficking pathway of the effector molecule.

Accordingly, the present application describes a conjugate comprising:
(a) a ligand that binds to a surface molecule on a target cell;
(b) an effector molecule; and
(c) a linker that couples the ligand and the effector molecule, the linker comprising at least one protease cleavage site corresponding to a protease found in an intracellular trafficking pathway of the effector molecule;
wherein cleavage of the linker by the protease uncouples the effector molecule from the ligand.

The invention also includes a pharmaceutical composition comprising a conjugate of the invention and a suitable diluent or carrier.

The invention further includes a method of diagnosing or treating a disease comprising administering an effective amount of a conjugate to a cell or animal in need thereof.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 is a schematic showing the nucleotide and amino acid sequences of VB6-845-F-de-bouganin-ALA70 (SEQ ID NOS:3 and 4).

FIG. 2 is a blot demonstrating the engineering of VB6-845-F-de-bouganin-Ala70. Supernatant of three independently induced VB6-845-F-de-bouganin-Ala70 clones transformed in E104 (lane 2, 3 and 4) were loaded under non-reducing conditions on a SDS-PAGE gel. Lane 1 corresponds to the induced parental clone of the Master Cell Bank. Lanes 5 and 6 correspond to the ladder and the supernatant of a non-induced culture, respectively. Lanes 7 and 8 were loaded with VB6-845-F-de-bouganin and VB6-845-F-gelonin, respectively. The nitrocellulose membrane was immunoblotted with a goat anti-human kappa light chain peroxidase conjugate antibody (1/1000). The upper arrow indicates the full-length protein at 65 kDa while the lower ones indicate the various truncated fragments of the immunocytotoxins.

FIG. 3 is a series of graphs showing reactivity of VB6-845-F-de-bouganin-Ala70. The reactivity and specificity of VB6-845-F-de-bouganin-Ala70 was assessed at 4° C. with Ep-CAM-positive cell lines, CAL-27 and MCF7 and Ep-CAM-negative cell line A375. A shift in median fluorescence with Ep-CAM-positive cell lines, CAL-27 and MCF7, was observed after incubation with VB6-845-F-de-bouganin-Ala70 (red line) and VB6-845-F-de-bouganin (green line). In contrast, no shift was observed with PBS (black line) as well as in A375 cell line.

FIG. 4 is a series of graphs showing internalisation of VB6-845-F-de-bouganin-Ala70. The internalisation of VB6-845-F-de-bouganin-Ala70 was assessed at 37° C. with Ep-CAM-positive cell lines, CAL-27 and MCF7. The pattern of internalisation of VB6-845-F-de-bouganin-Ala70 (red line) and VB6-845-F-de-bouganin (green line) are similar. No shift was observed with PBS (black line).

FIG. 5 is a series of graphs showing In vitro cytotoxicity of VB6-845-F-de-bouganin-Ala70. MTS assay of VB6-845-F-de-bouganin-Ala70 and VB6-845-F-de-bouganin with A375, CAL-27 and MCF7. A) A375 cells B) CAL-27 cells and C) MCF7 cells seeded at 5000 cells per well, were incubated with an equimolar concentration of VB6-845-F-de-bouganin-Ala70 (empty circles), VB6-845-F-de-bouganin (filled cones) and de-bouganin (filled circles) ranging from 500 to 0.5 nM. After 3 days incubation, the cell viability was measured and IC₅₀ determined.

FIG. 6 is a schematic showing nucleotide and amino acid sequences A:Nucleotide and amino acid sequences of VB6-845-F-gelonin (SEQ ID NOS: 9 and 10) and B: Nucleotide and amino acid sequences of VB6-845-F-PE (SEQ ID NOS: 11 and 12).

FIG. 7 is a series of images of showing VB6-845-F-de-bouganin-Ala70 colocalises with endosomal and lysosomal markers. CAL-27 cells were treated for 15, 30, 45, 60 and 120 min with 5 nM of VB6-845-F-de-bouganin-Ala70. These pictures are the representative view of five independent experiments. Following fixation and permeabilisation, CAL-27 cells were stained with anti-bouganin antibody and with antibodies for various subcellular markers: A) EEA1 (early endosome), B) LAMP-2 (late endosome/lysosomes). Scale bars, 10 μm.

FIG. 8 is a series of images showing localisation of VB6-845-F-gelonin in endosome and lysosome. CAL-27 cells were treated for 15, 30, 45, 60 and 120 min with 5 nM of VB6-845-F-gelonin. These pictures are the representative view of two independent experiments. Following fixation and permeabilisation, CAL-27 cells were stained with anti-gelonin and with antibodies for various subcellular markers: A) EEA1 (early endosome), B) LAMP-2 (late endosome/lysosomes). Scale bars, 10 μm.

FIG. 9 is a series of images VB6-845-F-de-bouganin-Ala70 weakly colocalises with the Golgi apparatus. CAL-27 cells were treated for 3 h with 5 nM of VB6-845-F-de-bouganin-Ala70, VB6-845-F-gelonin or VB6-845-F-PE. These pictures are the representative view of three to four independent experiments. Following fixation and permeabilisation, CAL-27 cells were stained with anti-bouganin, anti-gelonin or anti-PE antibodies, respectively, and with anti-p230 trans Golgi antibody, a marker of trans Golgi network. VB6-845-F-de-bouganin-Ala70 shows the weakest colocalisation with the trans Golgi network while VB6-845-F-PE shows the strongest. Scale bars, 10 μm.

FIG. 10 is a series of graphs showing the effect of alkalinisation of endosomal and lysosomal pH on VB6-845-F-de-bouganin cytotoxicity on CAL-27 cells. CAL-27 cells were exposed 3 days at 37° C. to serial logarithmic dilutions of cytotoxins and in the presence of 6.25 μM of chloroquine, 10 mM of NH₄Cl or 300 nM of monensin (empty circles, filled cones and empty triangles, respectively) or in the absence of any drugs (filled circles). Cell proliferation was assessed by MTS uptake. Results are expressed in percentage of viable cells compared to untreated cells. The dose-response curves are shown with standard deviations and are the representative example of two to four independent experiments. Cytotoxins used in this experiment were A) VB6-845-F-de-bouganin, B) de-bouganin, C) VB6-845-F-gelonin, D) gelonin, E) saporin, F) VB6-845-F-PE or G) ricin.

FIG. 11 is a series of graphs showing the effect of lactacystin on VB6-845-F-de-bouganin cytotoxicity on MCF7 cells. MCF7 cells were exposed 3 days at 37° C. to serial logarithmic dilutions of cytotoxins and in the presence of 5 or 10 μM of lactacystin (empty circles and filled cones, respectively) or in the absence of any drugs (filled circles). Cell proliferation was assessed by MTS uptake. Results are expressed in percentage of viable cells compared to cells un-treated. The dose-response curves are shown with standard deviations. Cytotoxins used in this experiment were A) VB6-845-F-de-bouganin, B) de-bouganin or C) VB6-845-F-PE.

FIG. 12 is a schematic showing the nucleotide and amino acids sequence of original VB6-845-F-de-bouganin (SEQ ID NOS:1 and 2) with linker sequences A through H (SEQ ID NOS:36 to 51) that are substituted in the conjugate replacing the original furin linker sequence. Nucleotide and amino acids numbering for the linkers indicates the changes that occur with the sequence substitution in the full immunoconjugate format.

FIG. 13 is a schematic showing nucleotide and amino acids sequence of nucleotide sequence optimized VB6-845-F-de-bouganin (SEQ ID NOS:52 and 53) with linker sequences A through G (SEQ ID NOS:76 to 89) that are substituted in the conjugate replacing the original furin linker sequence. Nucleotide and amino acids numbering for the linkers indicates the changes that occur with the sequence substitution in the full immunoconjugate format.

FIG. 14 is a western blot of VB6-845-F-Leg-de-bouganin VB6-845-F-CB-CD-de-bouganin and VB6-845-F-CB-CD-Leg-de-bouganin expression in culture supernatant. Supernatant of two independently induced cultures of clones transformed in E104 (lane 4 and 9) were loaded under non-reducing conditions on a SDS-PAGE gel. Lane 1 corresponds to the SeeBlueladder (Invitrogen™), supernatant of the induced culture of VB6-845-F-de-bouganin is on lane 3. Lane 2 corresponds to the VB6-845-F-de-bouganin purified product (100 ng/μL). The upper arrow indicates the full-length protein at 65 kDa while the lower ones indicate the various truncated fragments of the immunocytotoxins.

FIG. 15 is a graph showing quantification of Linkers expression by ELISA.

FIG. 16 is a flow cytometry assessment of binding of VB6-845-L-de-bouganin linker constructs to EpCAM positive (CAL-27) and negative (A-375) cell types in comparison to VB6-845-F-de-bouganin containing a furin site alone.

FIG. 17 is a series of graphs showing cytotoxicity of VB6-845-F-de-bouganin, VB6-845-Leg-de-bouganin, VB6-845-F-Leg-de-bouganin, VB6-845-CB-de-bouganin, VB6-845-CD-de-bouganin and VB6-845-F-CB-CD-de-bouganin on several EpCAM positive cell lines.

### DETAILED DESCRIPTION OF THE INVENTION

The application describes modified conjugates, such as immunotoxins, comprising a ligand, an effector molecule and a linker, which have improved cytotoxicity. The inventors have demonstrated that the inclusion of additional protease sensitive sites within the linker that correlate to proteases accessible or present in a trafficking pathway of the effector molecule, increases the efficacy and range of the conjugate, and increases the release of the effector molecule in a wider range of cell types.

### I. DEFINITIONS

The term “a cell” includes a single cell as well as a plurality or population of cells. Administering an agent to a cell includes both in vitro and in vivo administrations.

The term “administered systemically” as used herein means that the conjugate and/or other cancer therapeutic may be administered systemically in a convenient manner such as by injection (subcutaneous, intravenous, intramuscular, etc.), oral administration, inhalation, transdermal administration or topical application (such as topical cream or ointment, etc.), suppository applications, or means of an implant. An implant can be of a porous, non-porous, or gelatinous material, including membranes, such as sialastic membranes, or fibers. Suppositories generally contain active ingredients in the range of 0.5% to 10% by weight.

The term “amino acid” includes all of the naturally occurring amino acids as well as modified amino acids.

The term “antibody” as used herein is intended to include monoclonal antibodies, polyclonal antibodies, and chimeric antibodies. The antibody may be from recombinant sources and/or produced in transgenic animals. The term “antibody fragment” as used herein is intended to include without limitations Fab, Fab′, F(ab′)2, scFv, dsFv, ds-scFv, dimers, minibodies, diabodies, and multimers thereof, multispecific antibody fragments and Domain Antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques.

By “at least moderately stringent hybridization conditions” it is meant that conditions are selected which promote selective hybridization between two complementary nucleic acid molecules in solution. Hybridization may occur to all or a portion of a nucleic acid sequence molecule. The hybridizing portion is typically at least 15 (e.g. 20, 25, 30, 40 or 50) nucleotides in length. Those skilled in the art will recognize that the stability of a nucleic acid duplex, or hybrids, is determined by the Tm, which in sodium containing buffers is a function of the sodium ion concentration and temperature (Tm=81.5° C.−16.6 (Log 10 [Na+])+0.41 (% (G+C)−600/l), or similar equation). Accordingly, the parameters in the wash conditions that determine hybrid stability are sodium ion concentration and temperature. In order to identify molecules that are similar, but not identical, to a known nucleic acid molecule a 1% mismatch may be assumed to result in about a 1° C. decrease in Tm, for example if nucleic acid molecules are sought that have a >95% identity, the final wash temperature will be reduced by about 5° C. Based on these considerations those skilled in the art will be able to readily select appropriate hybridization conditions. In preferred embodiments, stringent hybridization conditions are selected. By way of example the following conditions may be employed to achieve stringent hybridization: hybridization at 5× sodium chloride/sodium citrate (SSC)/5×Denhardt's solution/1.0% SDS at Tm−5° C. based on the above equation, followed by a wash of 0.2×SSC/0.1°)/0.1% SDS at 60° C. Moderately stringent hybridization conditions include a washing step in 3×SSC at 42° C. It is understood, however, that equivalent stringencies may be achieved using alternative buffers, salts and temperatures. Additional guidance regarding hybridization conditions may be found in: Current Protocols in Molecular Biology, John Wiley & Sons, N.Y., 2002, and in: Sambrook et al., Molecular Cloning: a Laboratory Manual, Cold Spring Harbor Laboratory Press, 2001.

By “biologically compatible form suitable for administration in vivo” is meant a form of the substance to be administered in which any toxic effects are outweighed by the therapeutic effects.

The term “cancer” as used herein includes any cancer that can be bound by a ligand disclosed herein, preferably an antibody or antibody fragment disclosed herein.

The term “cancer cell” includes cancer or tumor-forming cells, transformed cells or a cell that is susceptible to becoming a cancer or tumor-forming cell.

The term “complementary” refers to nucleic acid sequences capable of base-pairing according to the standard Watson-Crick complementary rules, or being capable of hybridizing to a particular nucleic acid segment under stringent conditions.

A “conservative amino acid substitution” as used herein, is one in which one amino acid residue is replaced with another amino acid residue without abolishing the protein's desired properties.

The term “control” as used herein refers to a sample from a subject or a group of subjects who are either known as having cancer or not having cancer, or known as having a specific grade or severity of cancer.

The term “controlled release system” as used means the conjugate and/or other cancer therapeutic disclosed herein that can be administered in a controlled fashion. For example, a micropump may deliver controlled doses directly into the area of the tumor, thereby finely regulating the timing and concentration of the pharmaceutical composition (see, e.g., Goodson, 1984, in Medical Applications of Controlled Release, vol. 2, pp. 115-138).

The term “de-bouganin” as used here refers to a modified bouganin that has a reduced propensity to activate an immune response as described in PCT/CA2005/000410 and U.S. patent application Ser. No. 11/084,080, which published as US2005-0238642 A1. In one example, the modified bouganin has the amino acid sequence (SEQ ID NO: 90):

YNTVSFNLGEAYEYPTFIQDLRNELAKGTPVCQLPVTLQTIADDKRFVLV
DITTTSKKTVKVAIDVTDVYVVGYQDKWDGKDRAVFLDKVPTVATSKLFP
GVTNRVTLTFDGSYQKLVNAAKADRKALELGVNKLEFSIEAIHGKTINGQ
EAAKFFLIVIQMVSEAARFKYIETEWDRGLYGSFKPNFKVLNLENNWGDI
SDAIHKSSPQCTTINPALQLISPSNDPWVVNKVSQISPDMGILKFKSSK.

The term “derivative of a peptide” refers to a peptide having one or more residues chemically derivatized by reaction of a functional side group. Such derivatized molecules include for example, those molecules in which free amino groups have been derivatized to form amine hydrochlorides, p-toluene sulfonyl groups, carbobenzoxy groups, t-butyloxycarbonyl groups, chloroacetyl groups or formyl groups. Free carboxyl groups may be derivatized to form salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as derivatives are those peptides which contain one or more naturally occurring amino acid derivatives of the twenty standard amino acids. For examples: 4-hydroxyproline may be substituted for proline; 5-hydroxylysine may be substituted for lysine; 3-methylhistidine may be substituted for histidine; homoserine may be substituted for serine; and ornithine may be substituted for lysine.

The phrase “detecting or monitoring cancer” refers to a method or process of determining if a subject has or does not have cancer, the extent of cancer, the severity of cancer and/or grade of cancer.

The term “direct administration” as used herein means the cancer therapeutic may be administered, without limitation, intratumorally, intravascularly, and peritumorally. For example, the cancer therapeutic may be administered by one or more direct injections into the tumor, by continuous or discontinuous perfusion into the tumor, by introduction of a reservoir of the cancer therapeutic, by introduction of a slow-release apparatus into the tumor, by introduction of a slow-release formulation into the tumor, and/or by direct application onto the tumor. By the mode of administration “into the tumor,” introduction of the cancer therapeutic to the area of the tumor, or into a blood vessel or lymphatic vessel that substantially directly flows into the area of the tumor, is included.

As used herein, the phrase “effective amount” means an amount effective, at dosages and for periods of time necessary to achieve the desired result. Effective amounts of therapeutic may vary according to factors such as the disease state, age, sex, weight of the animal. Dosage regime may be adjusted to provide the optimum therapeutic response. For example, several divided doses may be administered daily or the dose may be proportionally reduced as indicated by the exigencies of the therapeutic situation.

The term “isolated nucleic acid sequences” as used herein refers to a nucleic acid substantially free of cellular material or culture medium when produced by recombinant DNA techniques, or chemical precursors, or other chemicals when chemically synthesized. An isolated nucleic acid is also substantially free of sequences which naturally flank the nucleic acid (i.e. sequences located at the 5′ and 3′ ends of the nucleic acid) from which the nucleic acid is derived. The term “nucleic acid” is intended to include DNA and RNA and can be either double stranded or single stranded, and represents the sense or antisense strand. Further, the term “nucleic acid” includes the complementary nucleic acid sequences.

The term “isolated polypeptides” refers to a polypeptide substantially free of cellular material or culture medium when produced by recombinant DNA techniques, or chemical precursors or other chemicals when chemically synthesized.

The term “nucleic acid sequence” as used herein refers to a sequence of nucleoside or nucleotide monomers consisting of naturally occurring bases, sugars and intersugar (backbone) linkages. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine.

The term “sample” as used herein refers to any fluid, cell or tissue sample from a subject which can be assayed.

The term “sequence identity” as used herein refers to the percentage of sequence identity between two polypeptide sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions.times.100%). In one embodiment, the two sequences are the same length. The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to a nucleic acid molecules of the present application. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The term “subject” as used herein refers to any member of the animal kingdom, preferably a mammal, more preferably a human being. In a preferred embodiment, the subject is suspected of having or has cancer.

As used herein, the phrase “treating or preventing cancer” refers to inhibiting of cancer cell replication, preventing transformation of a cell to a cancer-forming cell, inhibiting of cancer spread (metastasis), inhibiting of tumor growth, reducing cancer cell number or tumor growth, decreasing in the malignant grade of a cancer (e.g., increased differentiation), or improving cancer-related symptoms.

The term “variant” as used herein includes modifications or chemical equivalents of the amino acid and nucleic acid sequences disclosed herein that perform substantially the same function as the polypeptides or nucleic acid molecules disclosed herein in substantially the same way. For example, variants of polypeptides disclosed herein include, without limitation, conservative amino acid substitutions. Variants of polypeptides also include additions and deletions to the polypeptide sequences disclosed herein. In addition, variant des and variant nucleotide sequences include analogs and derivatives thereof. A variant of the cancer-associated antigen means a protein sequence that is expressed on or in cancer cells but not on or in normal cells or that is overexpressed on or in cancer cells relative to normal cells.

### II. CONJUGATES

The present application describes modified conjugates that comprise:
(a) a ligand that binds to a surface molecule on a target cell;
(b) an effector molecule; and
(c) a linker that couples the ligand and the effector molecule, the linker comprising at least one protease cleavage sequence corresponding to a protease found in an intracellular trafficking pathway of the effector molecule;

wherein cleavage of the linker by the protease uncouples the effector molecule from the ligand.

In one embodiment, the target cell is a cancer cell.

### a) Ligand

The term “ligand” as used herein refers to any molecule that binds a cell surface molecule on a target cell.

In one embodiment, the ligand is an antibody or antibody fragment. The antibody or fragment may be from any species including mice, rats, rabbits, hamsters and humans. Chimeric antibody derivatives, i.e., antibody molecules that combine a non-human animal variable region and a human constant region are also contemplated within the scope of the invention. Chimeric antibody molecules can include, for example, humanized antibodies which comprise the antigen binding domain from an antibody of a mouse, rat, or other species, with human constant regions. Conventional methods may be used to make chimeric antibodies. (See, for example, (Morrison et al., 1984); (Takeda et al., 1985), (Cabilly et al., 1989) U.S. Pat. No. 4,816,567; (Boss et al., 1989)., U.S. Pat. No. 4,816,397; (Taniguchi et al., 1986), European Patent Publication EP171496; (Morrison et al., 1986) European Patent Publication 0173494, (Neuberger and Rabbitts, 1989) United Kingdom patent GB 2177096B). The preparation of humanized antibodies is described in (Winter, 1987) EP-B1 239400. Humanized antibodies can also be commercially produced (Scotgen Limited, 2 Holly Road, Twickenham, Middlesex, Great Britain.). It is expected that chimeric antibodies would be less immunogenic in a human subject than the corresponding non-chimeric antibody. The humanized antibodies can be further stabilized for example as described in (Pluckthun et al., 2006) WO 00/61635.

The ligand may be immunoglobulin derived, i.e., can be traced to a starting molecule that is an immunoglobulin (or antibody). For example, the ligand may be produced by modification of an immunoglobulin scaffold using standard techniques known in the art. In another, non-limiting example, immunoglobulin domains (e.g., variable heavy and/or light chains) may be linked to a non-immunoglobulin scaffold. Further, the ligand may be developed by, without limitation, chemical reaction or genetic design.

The ligand need not be immunoglobulin based. Accordingly, a ligand may comprise a non-immunoglobulin polypeptide (e.g., Affibody®), or variant thereof. Such non-immunoglobulin polypeptide ligands can be designed to bind to a target cell surface molecule, such as a tumor associated molecule. Moreover, non-immunoglobulin polypeptide ligands can be engineered to a desired affinity or avidity, and can be designed to tolerate a variety of physical conditions, including extreme pH ranges and relatively high temperature.

Indeed, for use in a pharmaceutical composition, the design of a non-immunoglobulin polypeptide with a relatively long half-life at physiological conditions (e.g., 37° C. in the presence of peptidases) can be advantageous. Furthermore, such molecules, or variants thereof, may demonstrate good solubility, small size, proper folding and can be expressed in readily available, low-cost bacterial systems, and thus manufactured in commercially reasonable quantities. The ability to design a non-immunoglobulin polypeptide is within the skill of the ordinary artisan. See, e.g., U.S. Pat. Nos. 5,831,012 (Nilsson et al., 1998) and (Nilsson et al., 2003) 6,534,628 for techniques generally adaptable to design, manufacture, and select desired binding partners.

Examples of epitope-binding polypeptides include, without limitation, ligands comprising a fibronectin type III domain (see, e.g., International Publication Nos. (Lipovsek et al., 2001) WO 01/64942, (Lipovsek, 2000) WO 00/34784, (Lipovsek et al., 2002) WO 02/32925). Protein A-based affinity libraries have also been used to identify epitope-binding polypeptides (see, e.g., U.S. Pat. Nos. (Nilsson et al., 1998) 5,831,012 and (Nilsson et al., 2003) 6,534,628).

Other types of binding molecules are known in the art including, without limitation, binding molecules based on assembly of repeat protein domains (see, e.g., (Forrer et al., 2003), “A novel strategy to design binding molecules harnessing the modular nature of repeat proteins.” FEBS Lett. 539:2-6; (Kohl et al., 2003), “Designed to be stable: crystal structure of a consensus ankyrin repeat protein.” Proc Natl Acad Sci USA. 100:1700-1705).

Several non-immunoglobulin based, epitope-binding polypeptides and methods for making and using such polypeptides are known in the art (see, e.g., (Eklund et al., 2002) et al., 2002, “Anti-idiotypic protein domains selected from Protein A-based affibody libraries.” Prot. Struct. Funct. 48:454-462; (Gunneriusson et al., 1999), “Affinity maturation of a Taq DNA polymerase specific affibody by helix shuffling.” Prot. Eng. 12:873-878; (Hansson et al., 1999), “An in vitro selected binding protein (affibody) shows conformation-dependent recognition of the respiratory syncytial virus (RSV) G protein.” Immunotechnol. 4: 237-252; (Henning et al., 2002), “Genetic modification of adenovirus 5 tropism by a novel class of ligands based on a three-helix bundle scaffold derived from staphylococcal protein A.” Human Gene Therapy 13:1427-1439; (Hogbom et al., 2003), “Structural basis for recognition by an in vitro evolved affibody. Proc Natl Acad Sci USA. 100(6):3191-3196; (Nord et al., 1997), “Binding proteins selected from combinatorial libraries of an-helical bacterial receptor domain.” Nature Biotechnol. 15:772-777; (Nord et al., 2000), “Ligands selected from combinatorial libraries of protein A for use in affinity capture of apolipoprotein A-1M and Taq DNA polymerase.” J. Biotechnol. 80:45-54; (Nord et al., 1995), “A combinatorial library of an alpha-helical bacterial receptor domain.” Prot. Eng. 8:601-608; (Nord et al., 2001), “Recombinant human factor VIII-specific affinity ligands selected from phage-displayed combinatorial libraries of protein A.” Eur. J. Biochem. 268:1-10; (Nygren and Uhlen, 1997) “Scaffolds for engineering novel binding sites in proteins.” Curr. Opin. Struct. Biol. 7:463-469; (Ronnmark et al., 2002a), “Human immunoglobin A (IgA)-specific ligands from combinatorial engineering of protein A.” Eur. J. Biochem. 269:2647-2655; (Ronnmark et al., 2002b) et al., 2002, “Construction and characterization of affibody-Fc chimeras produced in Escherichia coli.” J. Immunol. Meth. 261:199-211; (Wahlberg et al., 2003), “An affibody in complex with a target protein: structure and coupled folding.” Proc Natl Acad Sci USA. 100(6):3185-3190; (Gotz et al., 2002), “Ultrafast electron transfer in the complex between fluorescein and a cognate engineered lipocalin protein, a so-called anticalin.” Biochemistry. 41:4156-4164; (Skerra, 2001), “Anticalins: a new class of engineered ligand-binding proteins with antibody-like properties.” J Biotechnol. 2001 74:257-275; (Skerra, 2000b), 2000, “Lipocalins as a scaffold.” Biochim Biophys Acta. 1482:337-350; (Skerra, 2000a), “Engineered protein scaffolds for molecular recognition.” J Mol. Recognit. 13:167-187; (Schlehuber et al., 2000), “A novel type of receptor protein, based on the lipocalin scaffold, with specificity for digoxigenin.” J Mol Biol. 297:1105-1120; (Beste et al., 1999), “Small antibody-like proteins with prescribed ligand specificities derived from the lipocalin fold.” Proc Natl Acad Sci USA. 96:1898-1903; (Bruhn et al., 1997) WO97/45538 entitled “Novel Synthetic Protein Structural Templates For The Generation, Screening And Evolution Of Functional Molecular Surfaces” (relating to production of libraries of peptide sequences in the framework of a structural template derived from Pleckstrin-Homology (PH) domains).

In a preferred embodiment, the ligand is an antibody or antibody fragment that binds to a cell surface molecule present on a cancer cell but not on normal cells. Examples of cancer or tumor associated antigens include, but are not limited to, Ep-CAM, CD44E (v8-10) (Glover et al., 2005), (PCTC/2005/000899), Scratch (Chahal et al., 2007) (PCTCA2005/000410) and Glut8 (Glover et al., 2006) (PCT/CA2005/001953) incorporated herein by reference. Preferably, the tumor associated antigen is Ep-CAM which is Epithelial Cell Adhesion Molecule and is also known as 17-1A, KSA, EGP-2 and GA733-2. Ep-CAM is highly expressed in many solid tumors including carcinomas of the bladder, lung, breast, ovary, colorectum and squamous cell carcinoma of the head and neck.

### b) Effector Molecule

The term “effector molecule” as used herein refers to any molecule that would be useful to deliver to a target cell, such as a cancer cell. In certain embodiments, the effector molecule activity is enhanced and/or requires cleavage from the ligand in order to exert its desired effect, namely cytotoxicity of target cells. In a preferred embodiment, the effector molecule is (i) a label, which can generate a detectable signal, directly or indirectly, or (ii) a therapeutic agent that one wishes to deliver to the cell. In a specific embodiment, the therapeutic agent is a cancer therapeutic agent, which is either cytotoxic, cytostatic or otherwise prevents or reduces the ability of the cancer cells to divide and/or metastasize.

One aspect provides a conjugate comprising (a) a ligand that binds to a cancer cell, preferably an antibody or antibody fragment, (b) a cancer therapeutic agent, such as a toxin, and (c) a linker that attaches the ligand and the cancer therapeutic; the linker comprising at least one protease cleavage sequence of a protease found in an intracellular trafficking pathway of the cancer therapeutic, wherein cleavage of the linker uncouples the effector molecule from the ligand.

In preferred embodiments, the cancer therapeutic agent is a toxin that comprises a polypeptide having ribosome-inactivating activity including, without limitation, gelonin, bouganin, saporin, ricin, ricin A chain, bryodin, diphtheria toxin, restrictocin, Pseudomonas exotoxin A, combinations, modified forms and variants thereof. In an embodiment, the effector molecule is a toxin and the conjugate is internalized by the cancer cell.

In one embodiment of the invention, the toxin is bouganin modified forms and/or variants thereof. In certain embodiments, the toxin is modified bouganin as described in the Examples. In a specific embodiment, modified bouganin is de-bouganin.

In another embodiment, the toxin is a truncated form of Pseudomonas exotoxin A that lacks the cell binding domain. For example, consists of amino acids 252-608.

In other nonlimiting embodiments, the cancer therapeutic agent comprises an agent that acts to disrupt DNA. Thus, the cancer therapeutic agents may be selected, without limitation, from enediynes (e.g., calicheamicin and esperamicin) and non-enediyne small molecule agents (e.g., bleomycin, methidiumpropyl-EDTA-Fe(II)). Other cancer therapeutic agents useful in accordance with the invention include, without limitation, daunorubicin, doxorubicin, distamycin A, cisplatin, mitomycin C, ecteinascidins, duocarmycin/CC-1065, and bleomycin/pepleomycin.

In other nonlimiting embodiments, the cancer therapeutic agent comprises an agent that acts to disrupt tubulin. Such agents may comprise, without limitation, rhizoxin/maytansine, paclitaxel, vincristine and vinblastine, colchicine, auristatin dolastatin 10 MMAE, and peloruside A.

In other nonlimiting embodiments, the cancer therapeutic portion may comprise an alkylating agent including, without limitation, Asaley NSC 167780, AZQ NSC 182986, BCNU NSC 409962, Busulfan NSC 750, carboxyphthalatoplatinum NSC 271674, CBDCA NSC 241240, CCNU NSC 79037, CHIP NSC 256927, chlorambucil NSC 3088, chlorozotocin NSC 178248, cis-platinum NSC 119875, clomesone NSC 338947, cyanomorpholinodoxorubicin NSC 357704, cyclodisone NSC 348948, dianhydrogalactitol NSC 132313, fluorodopan NSC 73754, hepsulfam NSC 329680, hycanthone NSC 142982, melphalan NSC 8806, methyl CCNU NSC 95441, mitomycin C NSC 26980, mitozolamide NSC 353451, nitrogen mustard NSC 762, PCNU NSC 95466, piperazine NSC 344007, piperazinedione NSC 135758, pipobroman NSC 25154, porfiromycin NSC 56410, spirohydantoin mustard NSC 172112, teroxirone NSC 296934, tetraplatin NSC 363812, thio-tepa NSC 6396, triethylenemelamine NSC 9706, uracil nitrogen mustard NSC 34462, and Yoshi-864 NSC 102627.

In other nonlimiting embodiments, the cancer therapeutic agent may comprise an antimitotic agent including, without limitation, allocolchicine NSC 406042, Halichondrin B NSC 609395, colchicine NSC 757, colchicine derivative NSC 33410, dolastatin 10 NSC 376128 (NG—auristatin derived), maytansine NSC 153858, rhizoxin NSC 332598, taxol NSC 125973, taxol derivative NSC 608832, thiocolchicine NSC 361792, trityl cysteine NSC 83265, vinblastine sulfate NSC 49842, and vincristine sulfate NSC 67574.

In other nonlimiting embodiments, the cancer therapeutic agent may comprise an topoisomerase I inhibitor including, without limitation, camptothecin NSC 94600, camptothecin, Na salt NSC 100880, aminocamptothecin NSC 603071, camptothecin derivative NSC 95382, camptothecin derivative NSC 107124, camptothecin derivative NSC 643833, camptothecin derivative NSC 629971, camptothecin derivative NSC 295500, camptothecin derivative NSC 249910, camptothecin derivative NSC 606985, camptothecin derivative NSC 374028, camptothecin derivative NSC 176323, camptothecin derivative NSC 295501, camptothecin derivative NSC 606172, camptothecin derivative NSC 606173, camptothecin derivative NSC 610458, camptothecin derivative NSC 618939, camptothecin derivative NSC 610457, camptothecin derivative NSC 610459, camptothecin derivative NSC 606499, camptothecin derivative NSC 610456, camptothecin derivative NSC 364830, camptothecin derivative NSC 606497, and morpholinodoxorubicin NSC 354646.

In other nonlimiting embodiments, cancer therapeutic agent may comprise an topoisomerase II inhibitor including, without limitation, doxorubicin NSC 123127, amonafide NSC 308847, m-AMSA NSC 249992, anthrapyrazole derivative NSC 355644, pyrazoloacridine NSC 366140, bisantrene HCL NSC 337766, daunorubicin NSC 82151, deoxydoxorubicin NSC 267469, mitoxantrone NSC 301739, menogaril NSC 269148, N,N-dibenzyl daunomycin NSC 268242, oxanthrazole NSC 349174, rubidazone NSC 164011, VM-26 NSC 122819, and VP-16 NSC 141540.

In other nonlimiting embodiments, the cancer therapeutic agent may comprise an RNA or DNA antimetabolite including, without limitation, L-alanosine NSC 153353, 5-azacytidine NSC 102816, 5-fluorouracil NSC 19893, acivicin NSC 163501, aminopterin derivative NSC 132483, aminopterin derivative NSC 184692, aminopterin derivative NSC 134033, an antifol NSC 633713, an antifol NSC 623017, Baker's soluble antifol NSC 139105, dichlorallyl lawsone NSC 126771, brequinar NSC 368390, ftorafur (pro-drug) NSC 148958, 5,6-dihydro-5-azacytidine NSC 264880, methotrexate NSC 740, methotrexate derivative NSC 174121, N-(phosphonoacetyl)-L-aspartate (PALA) NSC 224131, pyrazofurin NSC 143095, trimetrexate NSC 352122, 3-HP NSC 95678, 2′-deoxy-5-fluorouridine NSC 27640, 5-HP NSC 107392, alpha-TGDR NSC 71851, aphidicolin glycinate NSC 303812, ara-C NSC 63878, 5-aza-2′-deoxycytidine NSC 127716, beta-TGDR NSC 71261, cyclocytidine NSC 145668, guanazole NSC 1895, hydroxyurea NSC 32065, inosine glycodialdehyde NSC 118994, macbecin II NSC 330500, pyrazoloimidazole NSC 51143, thioguanine NSC 752, and thiopurine NSC 755.

In another nonlimiting embodiment, the therapeutic portion of the conjugate may be a nucleic acid. Nucleic acids that may be used include, but are not limited to, anti-sense RNA, genes or other polynucleotides, nucleic acid analogs such as thioguanine and thiopurine.

The present application further describes conjugates wherein the effector molecule is a label, which can generate a detectable signal, indirectly or directly. These conjugates can be used for research or diagnostic applications, such as for the in vivo detection of cancer. The label is preferably capable of producing, either directly or indirectly, a detectable signal. For example, the label may be radio-opaque or a radioisotope, such as ³H, ¹⁴C, ³²P, ³⁵S, ¹²³I, ¹²⁵I, ¹³¹I; a fluorescent (fluorophore) or chemiluminescent (chromophore) compound, such as fluorescein isothiocyanate, rhodamine or luciferin; an enzyme, such as alkaline phosphatase, beta-galactosidase or horseradish peroxidase; an imaging agent; or a metal ion.

### c) Linker

The term “linker” as used herein refers to a sequence, including nucleic acid sequence that encodes and amino acid sequence that comprises at least one protease cleavage site. The linker is optionally referred to herein as “L”.

A “protease cleavage site” is a sequence that is recognized and cleaved by a protease. For example, a furin cleavage site is cleaved by a furin protease, and a cathepsin B cleavage site is cleaved by a cathepsin B protease. A protease cleavage site may be recognized by more than one protease, including related proteases. A protease cleavage site for a specific protease is optionally referred to as the protease specific site, for example a “furin cleavage site” is alternatively referred to as a “furin specific site”. Additionally, a linker comprising a specific protease cleavage site is optionally referred to as a specific protease sensitive linker, for example a linker comprising a furin cleavage site is optionally referred to as a furin sensitive linker.

The linker sequence is chosen based on the trafficking pathway utilized by the effector molecule. In particular, the linker sequence will be cleavable by a protease that is present in an intracellular compartment in the cell where the conjugate is processed. Examples of such proteases include, but are not limited to furin, cathepsins, matrix metalloproteinases, and legumain.

In one embodiment, the linker includes multiple protease cleavage sites. In certain embodiments the linker comprises at least two protease cleavage sites. In one embodiment, the two protease cleavage sites are recognized by the same protease. In another embodiment, the two protease sites are recognized by different proteases. In other embodiments, the linker comprises three or more cleavage protease sites.

As mentioned above, the linker sequence is selected based on the trafficking pathway of the effector molecule. This is determined using techniques known in the art and methods disclosed herein. For conjugates trafficked through the endosomal/lysosomal system, a cathepsin protease specific linker is optionally used. For conjugates trafficked through the golgi apparatus, a furin specific linker is optionally used. For many of the common toxins much is known about their trafficking pathway and this knowledge is used to design the linker. For example, saporin and anthrax toxin traffick through the lysosome (Boquet et al., 1976) (Blaustein et al., 1989) (Vago et al., 2005). Accordingly, at least one protease cleavage site for a lysosome/endosome specific protease such as a cathepsin is comprised by the linker. Ricin, shiga toxin, and cholera toxin traffick through the Golgi apparatus (reviewed in (Sandvig and van Deurs, 2002), and (Spooner et al., 2006)). Accordingly, at least one protease cleavage site for a Golgi apparatus associated protease such as a furin is comprised by the linker.

The inventors have demonstrated that the therapeutic agent, bouganin and modified forms such as de-bouganin traffic through the lysosome and endosome. Inclusion of protease sites for endosomal and lysosomal proteases in the linker, increases the cytotoxicity of conjugates. Accordingly, in one embodiment, the effector molecule comprises bouganin, de-bouganin, or modified forms thereof; the linker comprises a protease cleavage site for an endosomal or lysosmal protease. In certain embodiments, the linker comprises a cathepsin specific site. In one embodiment, the cathepsin specific site is a cathepsin B specific site. In another embodiment the cathepsin specific site is a cathepsin D specific site. In other embodiments, the linker comprises at least two endosomal or lysosomal protease cleavage sites. In a preferred embodiment, the linker comprises a cathenpsin B specific site and a cathepsin D specific site.

In one embodiment, the linker comprises an amino acid sequence of SEQ ID NOS: 39, 41, 43, 45, 47, 49, 51, 79, 81, 83, 85, 87, or 89. In another embodiment, the linker consists of an amino acid sequence of SEQ ID NOS: 39, 41, 43, 45, 47, 49, 51, 79, 81, 83, 85, 87, or 89.

The application also discloses variants of the linker sequences disclosed above, including chemical equivalents. Such equivalents include proteins that perform substantially the same function as the specific proteins disclosed herein in substantially the same way. For example, the linker of SEQ ID NO:83 comprises a cathepsin B cleavage site. Thus, a variant of SEQ ID NO:83 will also be recognized and cleaved by cathespin B. For example, equivalents include, without limitation, conservative amino acid substitutions.

In one embodiment, the variant linker has at least 50%, preferably at least 60%, more preferably at least 70%, most preferably at least 80%, even more preferably at least 90%, and even most preferably 95% sequence identity to SEQ ID NOS: 39, 41, 43, 45, 47, 49, 51, 79, 81, 83, 85, 87, or 89.

The application also discloses the use of a nucleic acid sequence encoding the linkers disclosed herein. In one embodiment, the nucleic acid sequence encodes the linker having the amino acid sequence of SEQ ID NOS: 39, 41, 43, 45, 47, 49, 51, 79, 81, 83, 85, 87, or 89. In a further embodiment, the nucleic acid sequence encoding a linker comprises the nucleic acid sequence of SEQ ID NOS: 38, 40, 42, 44, 46, 48, 50, 78, 80, 82, 84, 86, or 88.

The application also discloses variants of the nucleic acid sequences that encode for a linker disclosed herein. For example, the variants include nucleotide sequences that hybridize to the nucleic acid sequences encoding a linker disclosed herein under at least moderately stringent hybridization conditions. In another embodiment, the variant nucleic acid sequences have at least 50%, preferably at least 70%, most preferably at least 80%, even more preferably at least 90% and even most preferably at least 95% sequence identity to SEQ ID NOS: 38, 40, 42, 44, 46, 48, 50, 78, 80, 82, 84, 86, or 88.

As the inventors have shown, if the trafficking pathway of the effector molecule is not already known it is determined using methods disclosed herein and techniques known in the art. An initial step for trafficking analysis of cytotoxic effectors is to render them inactive through, for example, a mutation in their active sites as described in (Morris and Wool, 1992) and (Rajamohan et al., 2000) or by using drug resistant cell types or through expression of the drug resistant gene as reviewed in (Tsuruo et al., 2003) and (Barrand et al., 1997), provided that, in the latter two, the trafficking pathway is not modified compared to normal susceptible cells. The inactivation of the toxin will prevent cell death that would render the analysis more difficult. If the effector molecule has a cell binding domain, the study can be conducted with the effector molecule alone, but trafficking should also be studied with the effector molecule attached to the ligand to form the conjugate of interest. After verification that the inactivating modification of the effector molecule does not alter its binding or internalization by the target cells through comparison with the active wild type by flow cytometry analysis, cells are then exposed to the effector and over time its position within the cell is visualized. Visualization can be done through standard immunohistochemical techniques as described in (John Wiley & Sons, 2006) Current Protocols in Cell Biology 2006 in which cells are co-incubated with antibodies specific to the effector molecule and antibodies specific to markers of cellular compartments (e.g. EEA1 for the endosome, LAMP-2 for the lysosome or p230 trans Golgi for the Golgi) attached to different detectable labels such as coloured fluorescent labels. The fluorescence is visualized microscopically and the compartments containing the effector are identified by the colocalization of the fluorescent indicators. Some effector molecules can also directly labelled with a detectable tag (ie fluorescent) as described in (Tavare et al., 2001). The localization of the effector molecule to cellular compartments indicates which trafficking pathway is being used, either endosome/lysosome or Golgi. To verify these results, cells that are sensitive to the effector molecule can be treated with agents that alter the environment in the compartment of interest. For example, agents that alter the pH of the endosome (e.g. chloroquine, monensin) or inhibit the proteasome linked to the Golgi pathway (e.g. lactacystin) can be used. Changes in the biological activity of an active form of the effector molecule on cells pre or co-incubated with these agents further indicates which cellular compartments are involved and therefore which trafficking pathway is used.

### d) Conjugate

In one embodiment, the conjugate comprises the amino acid sequence of SEQ ID NO:2 with a substitution at positions 489-500 with a linker consisting of the amino acid sequence of SEQ ID NOS: 39, 41, 43, 45, 47, 49, or 51. In another embodiment, the conjugate comprises the amino acid sequence of SEQ ID NO:53 with a substitution at positions 489-500 with a linker consisting of the amino acid sequence of SEQ ID NOS: 79, 81, 83, 85, 87, or 89.

The application also discloses variants of the conjugate sequences disclosed above, including chemical equivalents. Such equivalents include proteins that perform substantially the same function as the specific proteins disclosed herein in substantially the same way. For example, a variant of the conjugate comprising the amino acid sequence of SEQ ID NO:53 with a substitution at positions 489-500 with a linker consisting of the amino acid sequence of SEQ ID NO: 83 will have the same ligand binding function, effector function and protease cleavage site as the protein having the amino acid sequence of SEQ ID NO:53 with a substitution at positions 489-500 with a linker consisting of the amino acid sequence of SEQ ID NO: 83 (i.e. bind to Ep-CAM, has the function of de-bouganin, and is recognized and cleaved by cathespin B). For example, equivalents include, without limitation, conservative amino acid substitutions.

In one embodiment, the variant conjugate has at least 50%, preferably at least 60%, more preferably at least 70%, most preferably at least 80%, even more preferably at least 90%, and even most preferably 95% sequence identity to the amino acid sequence of SEQ ID NO:2 with a substitution at positions 489-500 with a linker consisting of the amino acid sequence of SEQ ID NOS: 39, 41, 43, 45, 47, 49, or 51 or SEQ ID NO:53 with a substitution at positions 489-500 with a linker consisting of the amino acid sequence of SEQ ID NOS: 79, 81, 83, 85, 87, or 89.

The application also discloses the use of a nucleic acid sequence encoding the conjugates disclosed herein. In one embodiment, the nucleic acid sequence encodes a conjugate comprising the amino acid sequence of SEQ ID NO:2 with a substitution at positions 489-500 with a linker consisting of the amino acid sequence of SEQ ID NOS: 39, 41, 43, 45, 47, 49, or 51. In another embodiment, the nucleic acid sequence encodes a conjugate comprising the amino acid sequence of SEQ ID NO:53 with a substitution at positions 489-500 with a linker consisting of the amino acid sequence of SEQ ID NOS: 79, 81, 83, 85, 87, or 89.

In another embodiment, the nucleic acid sequence comprises the nucleic acid sequence of SEQ ID NO:1 with a substitution at positions 1607-1642 with a nucleic acid sequence consisting of SEQ ID NO:38, 40, 42, 44, 46, 48 or 50. In a further embodiment, the nucleic acid sequence comprises the nucleic acid sequence of SEQ ID NO:52 with a substitution at positions 1557-1592 with a nucleic acid sequence consisting of SEQ ID NO:76, 78, 80, 82, 84, 86 or 88.

The application also discloses variants of the nucleic acid sequences that encode for a conjugate disclosed herein. For example, the variants include nucleotide sequences that hybridize to the nucleic acid sequences encoding a conjugate disclosed herein under at least moderately stringent hybridization conditions. In another embodiment, the variant nucleic acid sequences have at least 50%, preferably at least 70%, most preferably at least 80%, even more preferably at least 90% and even most preferably at least 95% sequence identity to the nucleic acid sequence comprising the nucleic acid sequence of SEQ ID NO:1 with a substitution at positions 1607-1642 with a nucleic acid sequence consisting of SEQ ID NO:38, 40, 42, 44, 46, 48 or 50 or the nucleic acid sequence comprising the nucleic acid sequence of SEQ ID NO:52 with a substitution at positions 1557-1592 with a nucleic acid sequence consisting of SEQ ID NO:76, 78, 80, 82, 84, 86 or 88.

In a specific embodiment, the conjugate comprises (a) an antibody or antibody fragment that binds to a cancer cell; (b) a cancer toxin; and (c) a linker comprising at least two protease cleavage sites. In one embodiment, the protease cleavage sites are selected from a furin specific site and a cathepsin specific site. In a specific embodiment, the cancer toxin is a bouganin or modified bouganin, preferably de-bouganin.

The ligand, preferably an antibody or antibody fragment, will be “attached to” or “coupled to” the effector molecule by the linker using techniques known in the art. For example, the ligand may be attached to the effector molecule through the linker by chemical or recombinant means. Chemical means for preparing fusions or conjugates are known in the art and can be used to prepare the conjugate. The method used to conjugate or couple the ligand and effector molecule through the linker must be capable of coupling the ligand with the effector molecule without interfering with the ability of the ligand to bind to the cell.

In certain embodiments, the conjugate is a protein fusion molecule. A ligand-linker-effector molecule protein fusion is optionally prepared using recombinant DNA techniques. A DNA sequence encoding the ligand protein is coupled to a DNA sequence encoding the linker sequence which is coupled to a DNA sequence encoding the effector molecule, resulting in a chimeric DNA molecule. The chimeric DNA molecule is transfected into a host cell and expresses the fusion protein. The fusion protein can be recovered from the cell culture and purified using techniques known in the art.

### III. PREPARATION OF PROTEINS

A person skilled in the art will appreciate that the polypeptides disclosed herein, such as the linkers and conjugates disclosed herein, may be prepared in any of several ways, but is most preferably prepared using recombinant methods.

Accordingly, the nucleic acid molecules disclosed herein may be incorporated in a known manner into an appropriate expression vector which ensures good expression of the polypeptides. Possible expression vectors include but are not limited to cosmids, plasmids, or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule and regulatory sequences selected on the basis of the host cells to be used for expression, which is operatively linked to the nucleic acid molecule. Operatively linked is intended to mean that the nucleic acid is linked to regulatory sequences in a manner which allows expression of the nucleic acid.

The application therefore includes a recombinant expression vector containing a nucleic acid molecule disclosed herein, or a fragment thereof, and the necessary regulatory sequences for the transcription and translation of the inserted protein-sequence.

Suitable regulatory sequences may be derived from a variety of sources, including bacterial, fungal, viral, mammalian, or insect genes (For example, see the regulatory sequences described in Goeddel, Gene Expression Technology Methods in Enzymology 185, Academic Press, San Diego, Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors may also contain a selectable marker gene which facilitates the selection of host cells transformed or transfected with a recombinant molecule disclosed herein. Examples of selectable marker genes are genes encoding a protein such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. Transcription of the selectable marker gene is monitored by changes in the concentration of the selectable marker protein such as β-galactosidase, chloramphenicol acetyltransferase, or firefly luciferase. If the selectable marker gene encodes a protein conferring antibiotic resistance such as neomycin resistance transformant cells can be selected with G418. Cells that have incorporated the selectable marker gene will survive, while the other cells die. This makes it possible to visualize and assay for expression of the recombinant expression vectors disclosed herein and in particular to determine the effect of a mutation on expression and phenotype. It will be appreciated that selectable markers can be introduced on a separate vector from the nucleic acid of interest.

The recombinant expression vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp., Melbourne, Australia), pMal (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors can be introduced into host cells to produce a transformed host cell. The terms “transformed with”, “transfected with”, “transformation” and “transfection” are intended to encompass introduction of nucleic acid (e.g. a vector) into a cell by one of many possible techniques known in the art. The term “transformed host cell” as used herein is intended to also include cells capable of glycosylation that have been transformed with a recombinant expression vector disclosed herein. Prokaryotic cells can be transformed with nucleic acid by, for example, electroporation or calcium-chloride mediated transformation. For example, nucleic acid can be introduced into mammalian cells via conventional techniques such as calcium phosphate or calcium chloride co-precipitation, DEAE-dextran mediated transfection, lipofectin, electroporation or microinjection. Suitable methods for transforming and transfecting host cells can be found in Sambrook et al. (Molecular Cloning: A Laboratory Manual, 3rd Edition, Cold Spring Harbor Laboratory Press, 2001), and other laboratory textbooks.

Suitable host cells include a wide variety of eukaryotic host cells and prokaryotic cells. For example, polypeptides disclosed herein may be expressed in yeast cells or mammalian cells. Other suitable host cells can be found in Goeddel, Gene Expression Technology: Methods in Enzymology 185, Academic Press, San Diego, Calif. (1990). In addition, the polypeptides disclosed herein may be expressed in prokaryotic cells, such as Escherichia coli (Zhang et al., Science 303(5656): 371-3 (2004)). In addition, a Pseudomonas based expression system such as Pseudomonas fluorescens can be used (US Patent Application Publication No. US 2005/0186666, Schneider, Jane C et al.).

Yeast and fungi host cells suitable for carrying out the methods disclosed herein include, but are not limited to Saccharomyces cerevisiae, the genera Pichia or Kluyveromyces and various species of the genus Aspergillus. Examples of vectors for expression in yeast S. cerevisiae include pYepSec1 (Baldari. et al., Embo J. 6:229-234 (1987)), pMFa (Kurjan and Herskowitz, Cell 30:933-943 (1982)), pJRY88 (Schultz et al., Gene 54:113-123 (1987)), and pYES2 (Invitrogen Corporation, San Diego, Calif.). Protocols for the transformation of yeast and fungi are well known to those of ordinary skill in the art (see Hinnen et al., Proc. Natl. Acad. Sci. USA 75:1929 (1978); Ito et al., J. Bacteriology 153:163 (1983), and Cullen et al. (Nat Bio/Tech 5:369 (1987)).

Suitable mammalian cells include, among others: COS (e.g., ATCC No. CRL 1650 or 1651), BHK (e.g. ATCC No. CRL 6281), CHO (ATCC No. CCL 61), HeLa (e.g., ATCC No. CCL 2), 293 (ATCC No. 1573) and NS-1 cells. Suitable expression vectors for directing expression in mammalian cells generally include a promoter (e.g., derived from viral material such as polyoma, Adenovirus 2, cytomegalovirus and Simian Virus 40), as well as other transcriptional and translational control sequences. Examples of mammalian expression vectors include pCDM8 (Seed, B., Nature 329:840 (1987)) and pMT2PC (Kaufman et al., EMBO J. 6:187-195 (1987)).

Given the teachings provided herein, promoters, terminators, and methods for introducing expression vectors of an appropriate type into plant, avian, and insect cells may also be readily accomplished. For example, within one embodiment, the polypeptides disclosed herein may be expressed from plant cells (see Sinkar et al., J. Biosci (Bangalore) 11:47-58 (1987), which reviews the use of Agrobacterium rhizogenes vectors; see also Zambryski et al., Genetic Engineering, Principles and Methods, Hollaender and Setlow (eds.), Vol. VI, pp. 253-278, Plenum Press, New York (1984), which describes the use of expression vectors for plant cells, including, among others, PAPS2022, PAPS2023, and PAPS2034).

Suitable insect cells include cells and cell lines from Bombyx, Trichoplusia or Spodotera species. Baculovirus vectors available for expression of proteins in cultured insect cells (SF 9 cells) include the pAc series (Smith et al., Mol. Cell. Biol. 3:2156-2165 (1983)) and the pVL series (Luckow, V. A., and Summers, M. D., Virology 170:31-39 (1989).

Alternatively, the polypeptides disclosed herein may also be expressed in non-human transgenic animals such as rats, rabbits, sheep and pigs (Hammer et al. Nature 315:680-683 (1985); Palmiter et al. Science 222:809-814 (1983); Brinster et al. Proc. Natl. Acad. Sci. USA 82:4438-4442 (1985); Palmiter and Brinster Cell 41:343-345 (1985) and U.S. Pat. No. 4,736,866).

The polypeptides disclosed herein may also be prepared by chemical synthesis using techniques well known in the chemistry of proteins such as solid phase synthesis (Merrifield, J. Am. Chem. Assoc. 85:2149-2154 (1964); Frische et al., J. Pept. Sci. 2(4): 212-22 (1996)) or synthesis in homogenous solution (Houbenweyl, Methods of Organic Chemistry, ed. E. Wansch, Vol. 15 I and II, Thieme, Stuttgart (1987)).

N-terminal or C-terminal fusion proteins comprising the polypeptides disclosed herein conjugated with other molecules, such as proteins may be prepared by fusing, through recombinant techniques. The resultant fusion proteins contain polypeptides disclosed herein fused to the selected protein or marker protein as described herein. The recombinant polypeptides disclosed herein may also be conjugated to other proteins by known techniques. For example, the proteins may be coupled using heterobifunctional thiol-containing linkers as described in WO 90/10457, N-succinimidyl-3-(2-pyridyldithio-proprionate) or N-succinimidyl-5 thioacetate. Examples of proteins which may be used to prepare fusion proteins or conjugates include cell binding proteins such as immunoglobulins, hormones, growth factors, lectins, insulin, low density lipoprotein, glucagon, endorphins, transferrin, bombesin, asialoglycoprotein glutathione-S-transferase (GST), hemagglutinin (HA), and truncated myc.

Accordingly, the application provides a recombinant expression vector comprising the nucleic acid sequences that encode the polypeptides disclosed herein, such as the linkers and conjugates disclosed herein. Further, the application provides a host cell comprising the nucleic acid sequences or recombinant expression vectors disclosed herein.

### IV. THERAPEUTIC METHODS AND PHARMACEUTICAL COMPOSITIONS

The inventors have shown that the conjugates disclosed herein show specificity for target cells, such as cancer cells. In addition, the inventors have shown that the conjugates disclosed herein are internalized by target cells. Thus, the conjugates disclosed herein can be used for the targeted delivery of bioactive or medically relevant agents, such as imaging, radioactive or cytotoxic agents.

One embodiment is a method of treating or preventing cancer, comprising administering to a subject having or suspected of having cancer an effective amount of a conjugate disclosed herein. Another embodiment is the use of an effective amount of a conjugate disclosed herein for the manufacture of a medicament for treating or preventing cancer. Furthermore, the application provides the use of an effective amount of a conjugate disclosed herein, further comprising the use of an additional cancer therapeutic agent for the manufacture of a medicament for simultaneous, separate or sequential treatment or prevention of cancer. The application also provides the use of an effective amount of a conjugate disclosed herein for treating or preventing cancer. Further, the application provides the use of an effective amount of a conjugate disclosed herein, further comprising the use of an additional cancer therapeutic agent for simultaneous, separate or sequential treatment or prevention of cancer.

In one embodiment, cancer includes, without limitation, stomach cancer, colon cancer, prostate cancer as well as cervical cancer, uterine cancer, ovarian cancer, pancreatic cancer, kidney cancer, liver cancer, head and neck cancer, squamous cell carcinoma, gastrointestinal cancer, breast cancer (such as carcinoma, ductal, lobular, and nipple), lung cancer, non-Hodgkin's lymphoma, multiple myeloma, leukemia (such as acute lymphocytic leukemia, chronic lymphocytic leukemia, acute myelogenous leukemia, and chronic myelogenous leukemia), brain cancer, neuroblastoma, sarcomas, rectum cancer, bladder cancer, pancreatic cancer, endometrial cancer, plasmacytoma, lymphoma, and melanoma. In one embodiment the cancer is colorectal cancer, breast cancer, ovarian cancer, pancreatic cancer, head and neck cancer, bladder cancer, gastrointestinal cancer, prostate cancer, liver cancer, renal cancer, melanomas, small cell and non small cell lung cancer, sarcomas, gliomas, or T- and B-cell lymphomas. In a further embodiment, the cancer is colon cancer, ovarian cancer, small cell lung cancer, prostate cancer or breast cancer. In another embodiment, the cancer is bladder cancer or head and neck squamous cell carcinoma.

The ability of a conjugate disclosed herein to selectively inhibit or destroy cells having cancer may be readily tested in vitro using cancer cell lines. The selective inhibitory effect of the conjugate disclosed herein may be determined, for example by demonstrating the selective inhibition of cellular proliferation of the cancer cells.

Toxicity may also be measured based on cell viability, for example, the viability of cancer and normal cell cultures exposed to the conjugate may be compared. Cell viability may be assessed by known techniques, such as trypan blue exclusion assays.

In another example, a number of models may be used to test the effectiveness of a conjugate disclosed herein. Thompson, E. W. et al. (Breast Cancer Res. Treatment 31:357-370 (1994)) has described a model for the determination of invasiveness of human breast cancer cells in vitro by measuring tumor cell-mediated proteolysis of extracellular matrix and tumor cell invasion of reconstituted basement membrane (collagen, laminin, fibronectin, Matrigel or gelatin). Other applicable cancer cell models include cultured ovarian adenocarcinoma cells (Young, T. N. et al. Gynecol. Oncol. 62:89-99 (1996); Moore, D. H. et al. Gynecol. Oncol. 65:78-82 (1997)), human follicular thyroid cancer cells (Demeure, M. J. et al., World J. Surg. 16:770-776 (1992)), human melanoma (A-2058) and fibrosarcoma (HT-1080) cell lines (Mackay, A. R. et al. Lab. Invest. 70:781 783 (1994)), and lung squamous (HS-24) and adenocarcinoma (SB-3) cell lines (Spiess, E. et al. J. Histochem. Cytochem. 42:917-929 (1994)). An in vivo test system involving the implantation of tumors and measurement of tumor growth and metastasis in athymic nude mice has also been described (Thompson, E. W. et al., Breast Cancer Res. Treatment 31:357-370 (1994); Shi, Y. E. et al., Cancer Res. 53:1409-1415 (1993)).

The conjugates may be formulated into pharmaceutical compositions for administration to subjects in a biologically compatible form suitable for administration in vivo. The substances may be administered to living organisms including humans, and animals. Administration of a therapeutically active amount of the pharmaceutical compositions is defined as an amount effective, at dosages and for periods of time necessary to achieve the desired result. For example, a therapeutically active amount of a substance may vary according to factors such as the disease state, age, sex, and weight of the individual, and the ability of the recombinant protein to elicit a desired response in the individual. Dosage regime may be adjusted to provide the optimum therapeutic response. For example, several divided doses may be administered daily or the dose may be proportionally reduced as indicated by the exigencies of the therapeutic situation.

Accordingly, the present application provides a pharmaceutical composition for treating or preventing cancer comprising the conjugate disclosed herein, and a pharmaceutically acceptable carrier, diluent or excipient. In a preferred embodiment, the effector molecule of the conjugate in the pharmaceutical composition is a cancer therapeutic agent, more preferably a toxin.

The pharmaceutical preparation comprising the conjugate may be administered systemically. The pharmaceutical preparation may be administered directly to the cancer site. Depending on the route of administration, the conjugate may be coated in a material to protect the compound from the action of enzymes, acids and other natural conditions that may inactivate the compound.

In accordance with one aspect of the present application, the conjugate is delivered to the patient by direct administration. The application contemplates the pharmaceutical composition being administered in at least an amount sufficient to achieve the endpoint, and if necessary, comprises a pharmaceutically acceptable carrier.

The application also provides methods for reducing the risk of post-surgical complications comprising administering an effective amount of the conjugate before, during, or after surgery to treat cancer.

The compositions described herein can be prepared by per se known methods for the preparation of pharmaceutically acceptable compositions that can be administered to subjects, such that an effective quantity of the active substance is combined in a mixture with a pharmaceutically acceptable vehicle. Suitable vehicles are described, for example, in (Gennaro, 2000) (Remington's Pharmaceutical Sciences (Remington's Pharmaceutical Sciences, 20th ed., Mack Publishing Company, Easton, Pa., USA, 2000). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include water, surfactants (such as Tween), alcohols, polyols, glycerin and vegetable oils, for example. Extemporaneous injection solutions and suspensions may be prepared from sterile powders, granules, tablets, or concentrated solutions or suspensions. conjugate may be supplied, for example but not by way of limitation, as a lyophilized powder which is reconstituted with sterile water or saline prior to administration to the patient.

Pharmaceutical compositions may comprise a pharmaceutically acceptable carrier. Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound, together with a suitable amount of carrier so as to provide the form for direct administration to the patient.

The composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

In various embodiments, the pharmaceutical composition is directly administered systemically or directly to the area of the tumor(s).

The pharmaceutical compositions may be used in methods for treating animals, including mammals, preferably humans, with cancer. The dosage and type of conjugate to be administered will depend on a variety of factors which may be readily monitored in human subjects. Such factors include the etiology and severity (grade and stage) of the cancer.

Clinical outcomes of cancer treatments using a conjugate disclosed herein are readily discernable by one of skill in the relevant art, such as a physician. For example, standard medical tests to measure clinical markers of cancer may be strong indicators of the treatment's efficacy. Such tests may include, without limitation, physical examination, performance scales, disease markers, 12-lead ECG, tumor measurements, tissue biopsy, cytoscopy, cytology, longest diameter of tumor calculations, radiography, digital imaging of the tumor, vital signs, weight, recordation of adverse events, assessment of infectious episodes, assessment of concomitant medications, pain assessment, blood or serum chemistry, urinalysis, CT scan, and pharmacokinetic analysis. Furthermore, synergistic effects of a combination therapy comprising a conjugate disclosed herein and another cancer therapeutic may be determined by comparative studies with patients undergoing monotherapy.

In the majority of approved cancer therapies, the cancer therapy is used in combination with other cancer therapies. Accordingly, the application provides a method of preventing or treating cancer using a conjugate disclosed herein in combination with at least one additional cancer therapy. The other cancer therapy may be administered prior to, overlapping with, concurrently, and/or after administration of the conjugate. When administered concurrently, the conjugate and the other cancer therapeutic may be administered in a single formulation or in separate formulations, and if separately, then optionally, by different modes of administration. The combination of one or more conjugates and one or more other cancer therapies may synergistically act to combat the tumor or cancer. The other cancer therapies include, without limitation, other cancer therapeutic agents including, without limitation, 2,2,2 trichlorotriethylamine, 3-HP, 5,6-dihydro-5-5-azacytidine, 5-aza-2′-deoxycytidine, 5-azacytidine, 5-fluorouracil, 5-HP, 5-propagermanium, 6-azauridine, 6-diazo-5-0×0-L-norleucine, 6-mercaptopurine, 6-thioguanine, abrin, aceglarone, acivicin, aclacinomycin, actinomycin, actinomycin D, aldesleukin, allocolchicine, allutamine, alpha-fetoprotein, alpha-TGDR, altretamine, aminocamptothecin, aminoglutethimide, aminopterin derivative, amonafide, amsacrine, an antifol, anastrozole, ancitabine, angiogenin antisense oligonucleotide, angiostatin, anthramycin, anthrapyrazole derivative, anti-thrombin, aphidicolin glycinate, ara-C, asparaginase, auristatin, autologous cells or tissues, avastin, azacitidine, azaserine, aziridine, AZQ, bacillus, baker's soluble antifol, batimastat, BCG live vaccine, bcl-2 antisense oligonucleotide, BCNU, benzodepa, betamethasone, beta-TGDR, biaomycin, bicalutamide, bisantrene, bleomycin, brequinar, buserelin, busulfan, cactinomycin, calicheamicin, calusterone, campath-1, camptothecin, camptothecin Na salt, capecitabine, carboplain, carboplatin, carboquone, carboxyphthalatoplatinum, carcinoembryonic antigen, carmofur, carmustine, camptothecin derivatives, carubicin, carzinophilin, CBDCA, CCNU, CHIP, chlorabusin, chlorambucil, chlormadinone acetate, chlornaphazine, chlorozotocin, chromomycins, cisplatin, cisplatinum, cladribine, clomesone, colchicine, colchicine derivative, collagen 14-amino acid peptide, cortisol, cortisone, cyanomorpholinodoxorubicin, cyclarabine, cyclocytidine, cyclodisone, cyclophosphamide, cyclothosphamide, cytarabine, cytochalasin B, cytosine arabinoside, dacarbazine, daclinomycin, dactinomycin, dasatinib, daunorubicin, defosfamide, dehydrotestosterone, demecolcine, denopterin, deoxydoxorubicin, dexamethasone, dianhydrogalactitol, diaziquone, dichlorallyl lawsone, diphtheria toxin, distamycin A, docetaxel, dolastatin 10, doxifluridine, doxorubicin, droloxifene, dromostanolone, duocarmycin/CC-1065, ecteinascidins, edatrexate, eflomithine, elliptinium acetate, emetine, emitefur, endostatin, enocitabine, epipodophyllotoxin, epirubicin, epitiostanol, erbitux, erlotinib, esperamicin, estramustine, estrogen, ethidium bromide, etoglucid, etoposide, fadrozole, fenretinide, fibronectin 29 kDa N-terminal proteolytic fragment, fibronectin 40 kDa C-terminal N-terminal proteolytic fragment, florafbr (pro-drug), floxuridhe, floxuridine, fludarabine, fluorodopan, flutamide, folinic acid, formestane, fosfestrol, fotemustine, gallium nitrate, gefitinib, gemcitabine, gemcitibine, gemtuzumab, glucocorticoid, goserelin, gramicidin D, granulocyte monocyte colony stimulating factor, guanazole NSC 1895, guerin, halichondrin B, hepsulfam, hexamethylmelamine, hexestrol, human chorionic gonadotropin, hycanthone, hydroxyurea, idarubicin, Ifosamide, imatinib, improsulfan, inosine glycodialdehyde, interferon, interferon-alpha, interferon-beta, interferon-gamma, interleukin-12, interleukin-15, interleukin-18, interleukin-1, interleukin-2, interleukin-2, interleukin-6, interleukins, irinotecan, iubidazone, kringle 5, L-alanosine, lapatinib, L-asparaginase, lauprolide acetate, lentinan, letrozole, leuprolide, leuprolide acetate (lupron), levamisole, lidocaine, liposomal dihydroxyanthracindione, lomusline, lomustine, lonidamine, lymphokines, lymphotoxin, lysodren, macbecin, macrophage inflammatory protein, m-AMSA, mannomustine, maytansine, mechlorethamine, mechlorethamine oxide hydrochloride, medroxyprogesterone, megestrol acetate, melanocyte lineage proteins, melengestrol, melphalan, menogaril, mepitiostane, mercaptopurine, mesna, methidiumpropyl-EDTA-Fe(I1)), methotrexate, methotrexate derivative, meturedepa, miboplatin, miltefosine, mineral corticoid, mithramycin, mitobronitol, mitoguazone, mitolactol, mitolanc, mitomycin C, mitotane, mitoxantrone, mitozolamide, mopidamol, morpholinodoxorubicin, mutated tumor-specific antigens, mycophenolic acid, N-(phosphonoacety1)-L-aspartate (PALA), N,N-dibenzyl daunomycin, nerve growth factor, nilotinib, nilutamide, nimustine, nitracine, nitrogen mustard, nogalamycin, nonautologous cells or tissues, novembichin, olivomycins, ontak, onyx-015, oxaliplatin, oxanthrazole, paclitaxel, PCNU, pegaspergase, pelomside A, pentoslatin, peplomycin, perfosfamide, phenamet, phenesterine, picamycin, piperazine, piperazinedione, pipobroman, piposulfan, pirarubicin, piritrexim, platelet derived growth factor, platelet factor-4 7.8 kDa proteolytic fragment, platelet factor-4 13 amino acid peptide, plicamycin, podophyllinic acid 2-ethyl-hydrazide, podophyllotoxin, polyestradiol phosphate, porfimir, porfiromycin, prednimustine, prednisone, procabazine, procaine, progestine, prolactin 16 kDa proteolytic fragment, propranolol, pseudomonas exotoxin, PSK, pteropterin, puromycin, pyrazofurin, pyrazoloacridine, pyrazoloimidazole, ranimustine, razoxane, retinoid, rhizoxin, rhizoxinlmaytansine, ricin A, rituxan, rituximab, riuxlmab, roquinimex, serpin (serine protease inhibitor), sizofican, sobuzoxane, sorafenib, SPARC, 20-amino acid peptide, spirogermanium, spirohydantoin mustard, straplozocin, streptonigrin, streptozocin, sunitinib, tamoxifen, taxol, taxol derivative, tegafur, temozoamide, teniposide, tenuazonic acid, teroxirone, testolactone, tetracaine, tetraplatin, thalidomide, thiamiprine, thiocolchicine, thioepa, thiopurine, thio-tepa, thrombospondin I 19 amino acid peptide, tissue plasminogen activator, tomudex, topotecan, toremifene, trastuzutmaban, tretinoin, triaziquone, triethylenemelamine, triethylenephosphoramide, triethylenethiophosphoramide, trilostane, trimetrexate, triptorelin, trityl cysteine, trofosfamide, trontecan, tubercidin, tumor necrosis factor-like cytokine, tumor necrosis factors, ubenimex, uracil mustard, uracil nitrogen mustard, uredepa, urethan, vandetanib (ZD6474), VEGF antisense oligonucleotide, vinblastine, vinblastine sulfate, vincristine, vincristine sulfate, vindesine, vinorelbine, VM-26, VP-16, yoshi-864, zinostatin and/or zorubicin.

In another embodiment, one or more conjugate disclosed herein can be administered in combination with one or more of the following cancer therapies or categories of therapeutic agents, including without limitation, radiation, surgery, gene therapy, agents to control of side effects (eg. antihistaminic agents, anti-nausea agents), cancer vaccines, inhibitors of angiogenesis, immune modulators, anti-inflammatories, immunosuppressants, agents that increase expression of antigen, other agents associated with cancer therapy, chemotherapeutic agents, immunotherapeutics, photosensitizers, tyrosine kinase inhibitors, antibiotics, antimetabolites, agents that acts to disrupt DNA, agents that acts to disrupt tubulin, alkylating agents, topoisomerase I inhibitors, topoisomerase II inhibitors, cytokines, growth factors, hormonal therapies, vinca alkyloids, plant alkaloids, and/or anti-mitotic agents.

The inventors have shown that lactone-containing proteasome inhibitors improve the cytotoxicity of conjugates that traffic through the ER-associated degradation pathway (ERAD), such as conjugates comprising Pseudomonas exotoxin A or a truncated form of Pseudomonas exotoxin A that consists of amino acids 252-608, or variants thereof. Thus, in one embodiment the conjugate is processed through the ERAD pathway and the additional cancer therapeutic comprises a lactone-containing proteasome inhibitor or an analog thereof. For example, lactone-containing proteasome inhibitors include, without limitation, epoxomicin, MG-132, lactacystin, trichostatin A, curcumin, proteasome inhibitor I, chymostatin, lovastatin, simvastatin, FTI-277, GGTI-298, ascorbic acid, acetylsalicyclic acid and butyrolactone (See Efuet and Keomarsi, 2006; Roa, S. et al., 1999). In a further embodiment, the conjugate that is processed through the ERAD pathway comprises Pseudomonas exotoxin A as the effector molecule.

Indeed, administration of an effective amount of a conjugate to a patient in need of such treatment may result in reduced doses of another cancer therapeutic having clinically significant efficacy. Such efficacy of the reduced dose of the other cancer therapeutic may not be observed absent administration with a conjugate. Accordingly, the present application provides methods for treating a tumor or cancer comprising administering a reduced dose of one or more other cancer therapeutics.

Moreover, combination therapy comprising a conjugate to a patient in need of such treatment may permit relatively short treatment times when compared to the duration or number of cycles of standard treatment regimens. Accordingly, the present application provides methods for treating a tumor or cancer comprising administering one or more other cancer therapeutics for relatively short duration and/or in fewer treatment cycles.

Thus, in accordance with the present application, combination therapies comprising a conjugate disclosed herein and another cancer therapeutic may reduce toxicity (i.e., side effects) of the overall cancer treatment. For example, reduced toxicity, when compared to a monotherapy or another combination therapy, may be observed when delivering a reduced dose of conjugate and/or other cancer therapeutic, and/or when reducing the duration of a cycle (i.e., the period of a single administration or the period of a series of such administrations), and/or when reducing the number of cycles.

Accordingly, the application provides a pharmaceutical composition comprising a conjugate disclosed herein and one or more additional anticancer therapeutic, optionally in a pharmaceutically acceptable carrier.

The present application also provides a kit comprising an effective amount of a conjugate disclosed herein, optionally, in combination with one or more other cancer therapeutic, together with instructions for the use thereof to treat cancer. The kit can also include ancillary agents. For example, the kits can include instruments for injecting the conjugate into a subject, such as a syringe; vessels for storing or transporting the conjugate; and/or pharmaceutically acceptable excipients, carriers, buffers or stabilizers.

As stated above, combination therapy with the conjugate may sensitize the cancer or tumor to administration of an additional cancer therapeutic. Accordingly, the present application contemplates combination therapies for preventing, treating, and/or preventing recurrence of cancer comprising administering an effective amount of a conjugate prior to, subsequently, or concurrently with a reduced dose of a cancer therapeutic. For example, initial treatment with a conjugate may increase the sensitivity of a cancer or tumor to subsequent challenge with a dose of cancer therapeutic. This dose is near, or below, the low range of standard dosages when the cancer therapeutic is administered alone, or in the absence of a conjugate. When concurrently administered, the conjugate may be administered separately from the cancer therapeutic, and optionally, via a different mode of administration.

In an alternate embodiment, administration of the additional cancer therapeutic may sensitize the cancer or tumor to the conjugate. In such an embodiment, the additional cancer therapeutic may be given prior to administration of the conjugate.

Combination therapy may thus increase the sensitivity of the cancer or tumor to the administered conjugate and/or additional cancer therapeutic. In this manner, shorter treatment cycles may be possible thereby reducing toxic events. The cycle duration may vary according to the specific cancer therapeutic in use. The application also contemplates continuous or discontinuous administration, or daily doses divided into several partial administrations. An appropriate cycle duration for a specific cancer therapeutic will be appreciated by the skilled artisan, and the application contemplates the continued assessment of optimal treatment schedules for each cancer therapeutic. Specific guidelines for the skilled artisan are known in the art. See, e.g., Therasse et al., 2000, “New guidelines to evaluate the response to treatment in solid tumors. European Organization for Research and Treatment of Cancer, National Cancer Institute of the United States, National Cancer Institute of Canada,” J Natl Cancer Inst. February 2; 92(3):205-16.

It is contemplated that the conjugate may be administered by any suitable method such as injection, oral administration, inhalation, transdermal or intratumorally, whereas any other cancer therapeutic may be delivered to the patient by the same or another mode of administration. Additionally, where multiple cancer therapeutics are intended to be delivered to a patient, the conjugate and one or more of the other cancer therapeutics may be delivered by one method, whereas other cancer therapeutics may be delivered by another mode of administration.

### (G) Diagnostic Methods and Agents

The conjugates disclosed herein bind selectively to target cell, such as cancer cells. Therefore the conjugates can be used in the diagnosis of cancer.

Accordingly, the present application includes diagnostic methods, agents, and kits that can be used by themselves or prior to, during or subsequent to therapeutic methods in order to determine whether or not cancer cells are present.

In one embodiment, the application provides a method of detecting or monitoring cancer in a subject comprising the steps of
(1) contacting a test sample from said subject with a conjugate disclosed herein and that binds specifically to a cancer cell to produce a conjugate-antigen complex;
(2) measuring the amount of conjugate-antigen complex in the test sample; and
(3) comparing the amount of conjugate-antigen complex in the test sample to a control.

The application further includes a kit for diagnosing cancer comprising any one of the conjugates disclosed herein and instructions for the use thereof to diagnose the cancer. The kit can also include ancillary agents. For example, the kits can include additional reagents, such as agents to detect the conjugates disclosed herein directly or indirectly; vessels for storing or transporting the conjugates; positive and/or negative controls or reference standards; and/or other buffers or stabilizers.

For use in the diagnostic applications, the effector molecule is preferably a label as described above.

The above disclosure generally describes the present invention. A more complete understanding can be obtained by reference to the following specific examples. These examples are described solely for the purpose of illustration and are not intended to limit the scope of the invention. Changes in form and substitution of equivalents are contemplated as circumstances might suggest or render expedient. Although specific terms have been employed herein, such terms are intended in a descriptive sense and not for purposes of limitation.

### EXAMPLES

The following non-limiting examples are illustrative of the present invention:

### Example 1

### Engineering an Inactive Cytotoxin

### Site Directed Mutagenesis to Engineer VB6-845-F-de-bouganin-Ala70

VB6-845-F-de-bouganin (SEQ ID NOS: 1 and 2) is an immunotoxin comprising a Fab that binds to Ep-CAM linked to the toxin bouganin through a furin specific linker (F) (SEQ ID NOS: 36 and 37) from which T-cell epitopes have been removed, namely de-bouganin. To allow for the visualization of the VB6-845-F-de-bouganin-Ala70 immunotoxin (SEQ ID NO: 3 and 4) within the cells an inactive version was created containing a point mutation of tyrosine 70 to alanine.

The primers, purchased from Invitrogen™, were:

Primer #1, 5′-SacI-C_{L}-boug,

(SEQ ID NO: 5)

(5′-TACGCCTGCGAAGTCACCCATCAGGGCCTGAGCTCGCCCGTC-3′)
Primer #2, boug-Ala70-3′,

(SEQ ID NO: 6)

(5′-GTCTTGATAACCCACAACTGCCACATCTGTCAC-3′)
to engineer SacI-C_{L}-F-boug-Ala70.
Primer #3, 5′-boug-Ala7o,

(SEQ ID NO: 7)

(5′-GTGACAGATGTGGCAGTTGTGGGTTATCAA-3′)
Primer #4, stop-XhoI-3′,

(SEQ ID NO: 8)

(5′-CTCGAGCTACTATTTGGAGCTTTTAAACTTAAGGATACC-3′)
to engineer boug-Ala70-stop-XhoI.

Primer #2 and primer #3 contain the codon encoding the Tyr70→Ala70 mutation.

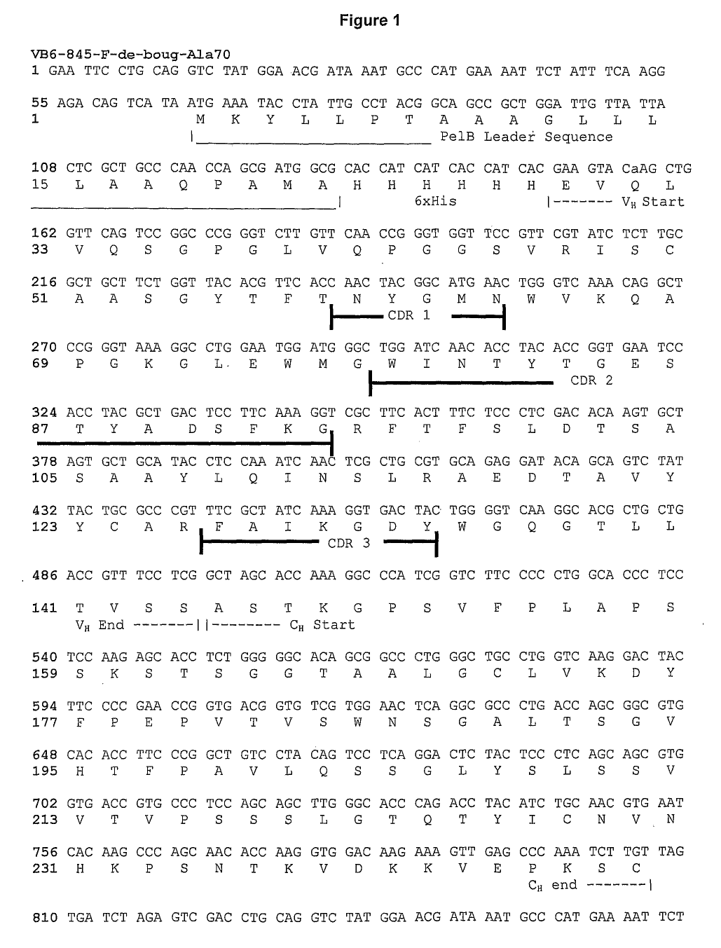

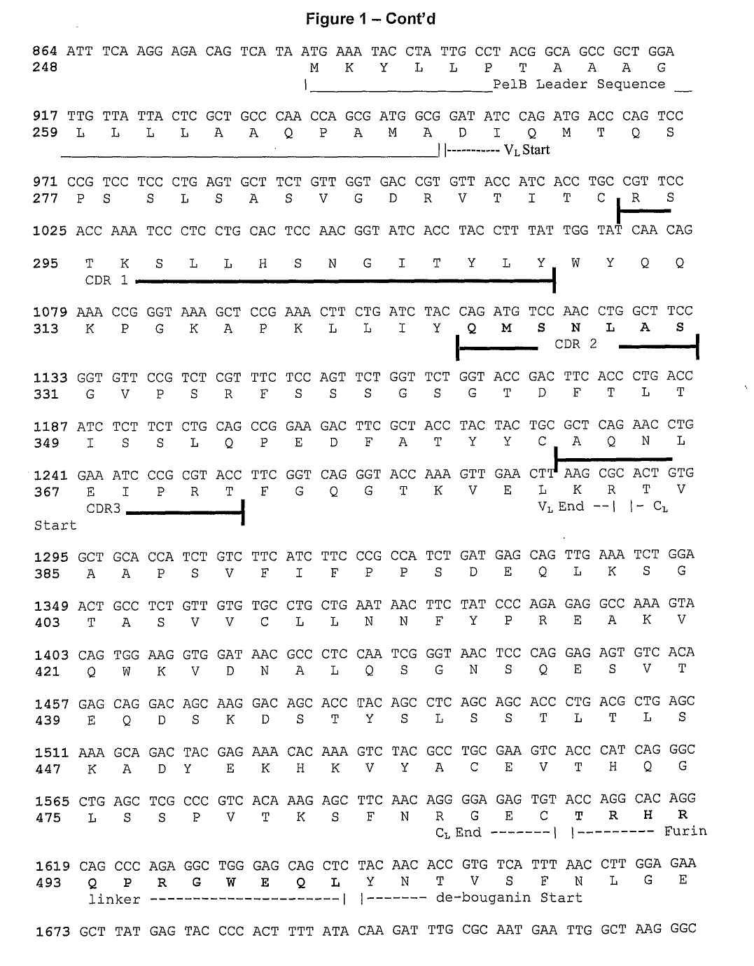

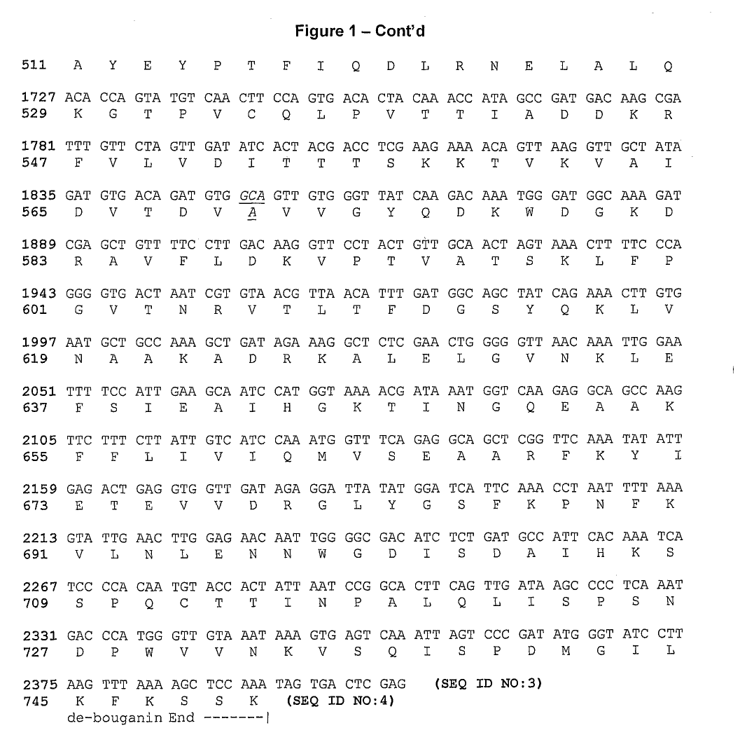

Using VB6-845-F-de-bouganin/pSP73 plasmid as a template, SacI-C_{L}-F-boug-Ala70 and boug-Ala70-stop-XhoI fragments were amplified by Polymerase Chain Reaction (PCR) with the pair of primers #1 and #2 and the pair of primers #3 and #4, respectively. These two fragments were linked through splice overlap extension PCR using external primers #1 and #4 This PCR fragment, SacI-boug-Ala70-XhoI, was purified using QIAquick® Gel Extraction Kit (QIAGEN), inserted into pCR®2.1-TOPO® vector using TOPO TA Cloning® Kit (1:10 ratio) and transformed into chemically competent E. coil 10F cells (selection on Luria-Bertani [LB] agar kanamycin plate, 100 μg/mL). Plasmids of four positive clones were purified using QIAprep® Spin Miniprep kit (QIAGEN) and sequenced using CEQ™ 8000 Genetic Analysis System (Beckman Coulter) as described below. A SacI-boug-Ala70-XhoI/pCR®2.1 clone with a verified sequence was digested with SacI and XhoI (Promega and Invitrogen™, respectively) and purified products of the digestion were ligated with VB6-845-F-de-bouganin/pSP73 predigested with the same enzymes to engineer VB6-845-F-de-bouganin-Ala70/pSP73. VB6-845-F-de-bouganin-Ala70 was digested with EcoRI and XhoI (Invitrogen™) and cloned into pING3302 vector pre-digested with the same restriction enzymes. The resultant, VB6-845-F-de-bouganin-Ala70/pING3302, was transformed into chemically competent E. coli 10F cells (selection on LB agar tetracylcine plate, 25 μg/mL). The extracted VB6-845-F-de-bouganin-Ala70/pING3302 plasmid was then transformed into chemically competent E. coli E104 cells using the previously described CaCl₂ method and sequenced using CEQ™ 8000 Genetic Analysis System (Beckman Coulter) as described below. The nucleotide sequence and amino acid sequences (SEQ ID NO: 3 and 4) of the VB6-845-F-de-bouganin-Ala70 inactive construct are shown in FIG. 1.

### Small-Scale Expression of VB6-845-F-de-bouganin-Ala70

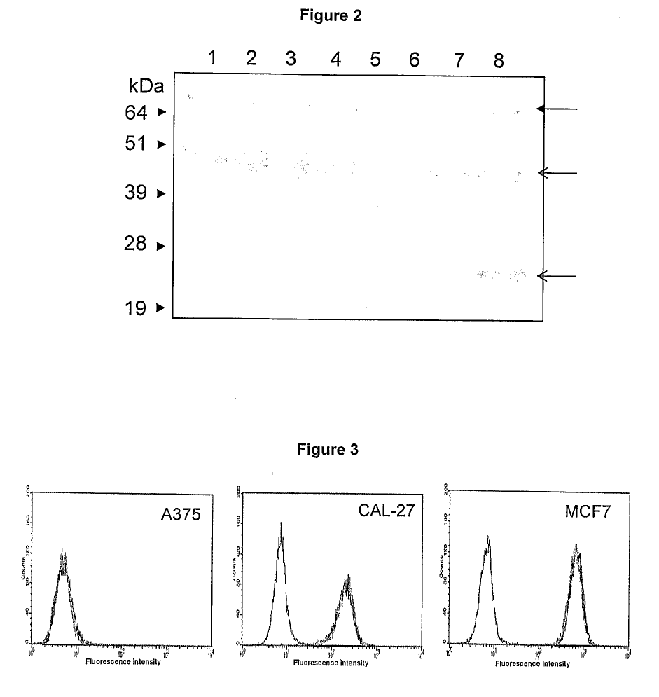

The Western blot analysis under non-reducing conditions of the VB6-845-F-de-bouganin-Ala70 clone showed that a full-length protein, similar to VB6-845-F-de-bouganin was detected with a goat anti-human kappa light chain peroxidase conjugate antibody (FIG. 2, lane 1 and 7). In addition, truncated products with similar size were detected for both constructs. The Western blotting of non-induced E104 culture supernatant revealed no corresponding bands indicating that these proteins are specifically detected with the goat anti-human kappa light chain peroxidase conjugate antibody (FIG. 2, lane 6). To validate the consistency of the master cell bank, size and expression level of the product of three different clones were assessed (FIG. 2, lanes 2 to 4).

### Biological Characterisation of VB6-845-F-de-bouganin-Ala70

EpCam Specific Cell Binding

For the measurement of cell surface Ep-CAM binding, cells (typically 0.3×10⁶ in 2 mL) were resuspended in flow cytometry buffer (10% foetal bovine serum in PBS) and incubated with 130 nM of controls and test antibodies and in presence or absence of 6.25 μM chloroquine for 2 h at 4° C. to prevent internalisation during the staining procedure. After washing twice with the same buffer, cells were incubated with the second step reagent, rabbit anti-bouganin (1:100 dilution in flow cytometry buffer) for an additional 1 h at 4° C. After two washes in 10% foetal bovine serum in PBS, a third step reagent, goat anti-rabbit FITC conjugate immunoglobulin (1:100 dilution in flow cytometry buffer) was added to detect bound rabbit anti-bouganin to VB6-845-F-de-bouganin and VB6-845-F-de-bouganin-Ala70. To measure internalisation, half of the sample was analysed on a BD FACSCalibur™ cytometer while the other half was incubated at 37° C., 5% CO₂ for 1 h followed by a wash and a 1 h incubation at 4° C.

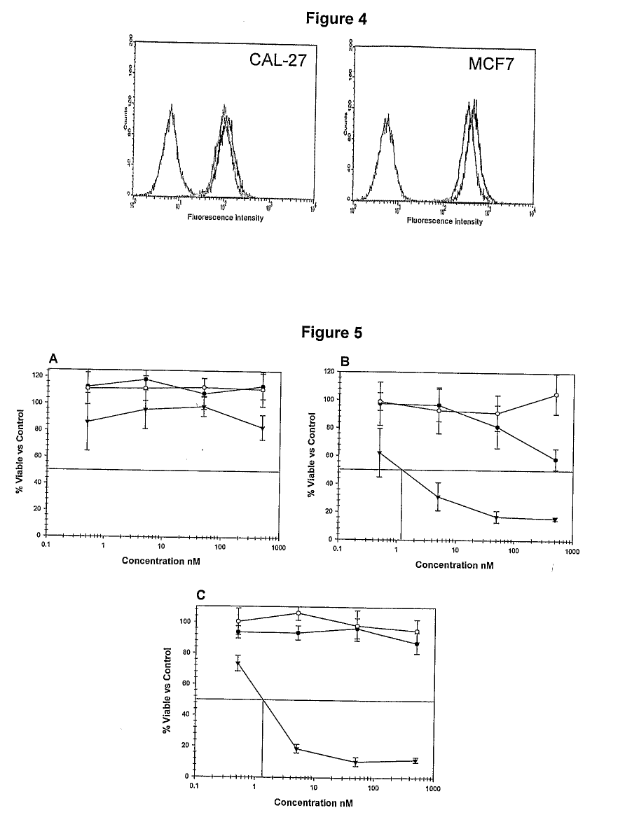

VB6-845-F-de-bouganin binds to the Ep-CAM-positive cell lines, CAL-27 and MCF7 but not to the Ep-CAM-negative cell line, A375. The VB6-845-F-de-bouganin-Ala70 binding activity was compared to VB6-845-F-de-bouganin by flow cytometry at 130 nM. A shift in median fluorescence was observed with both VB6-845-F-de-bouganin proteins incubated at 4° C. with CAL-27 and MCF7 (FIG. 3). Internalisation at 37° C. of VB6-845-F-de-bouganin is 1.4 and 1.9 better than VB6-845-F-de-bouganin-Ala70 for CAL-27 and MCF7, respectively, though both immunocytotoxins show a similar pattern of internalisation (FIG. 4).

Cell Viability Assay

Cells, seeded at 5000 cells per well in flat bottomed 96-well plates (Nunc™), were treated with the appropriate drug and/or cytotoxin in triplicate wells. The plates were then incubated at 37° C. in a 5% CO₂-supplemented atmosphere for 3 days. Cell viability was assessed by reduction of the tetrazolium salt (MTS) to the formazan product by dehydrogenase enzymes found in metabolically active cells (CellTiter 96® Aqueous Non-Radioactive Cell Proliferation Assay, Promega) as described (Mosmann, 1983 and Denizot and Lang, 1986). The data were expressed as a percentage of absorbance at 490 nm observed in untreated cells.

CAL-27, MCF7 and A375 cell lines were incubated with an equimolar concentration of VB6-845-F-de-bouganin-Ala70, VB6-845-F-de-bouganin and de-bouganin ranging from 500 nM to 0.5 nM. After 3 days of incubation, the calculated IC₅₀ of VB6-845-F-de-bouganin was approximately 1.5 nM with CAL-27 and MCF7 (FIGS. 5B and 5C). For comparison, no IC₅₀ was measured with VB6-845-F-de-bouganin-Ala70 and de-bouganin with both cell lines. No cytotoxicity was observed with A375 cells for either cytotoxin (FIG. 5A).

The above experiments verify that while the VB6-845-F-de-bouganin-Ala70 maintains binding and internalization functions through Ep-CAM, it is no longer toxic to the cells and allows for the tracking of the de-bouganin trafficking.

### Example 2

### Mapping Intracellular Trafficking Pathways of Effectors

Colocalisation of VB6-845-F-de-bouganin-Ala70 with Intracellular Markers

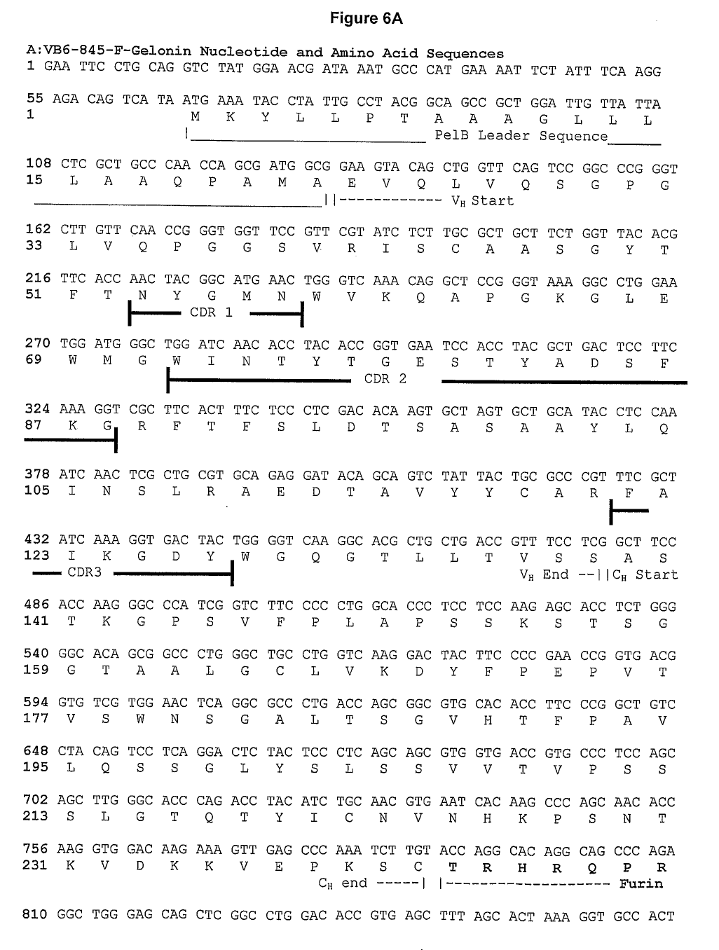

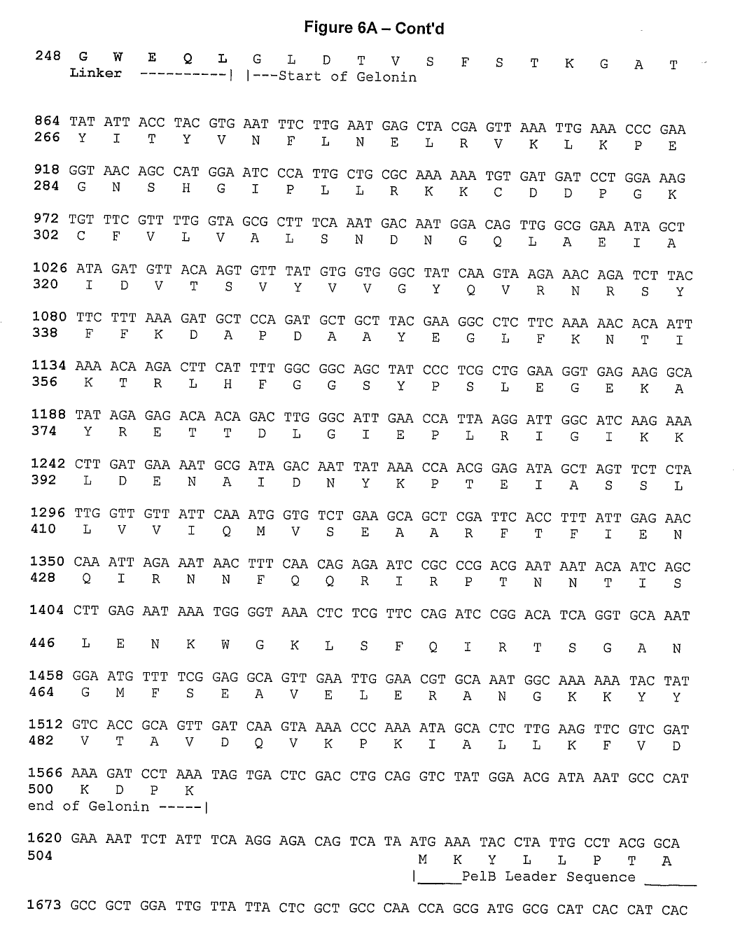

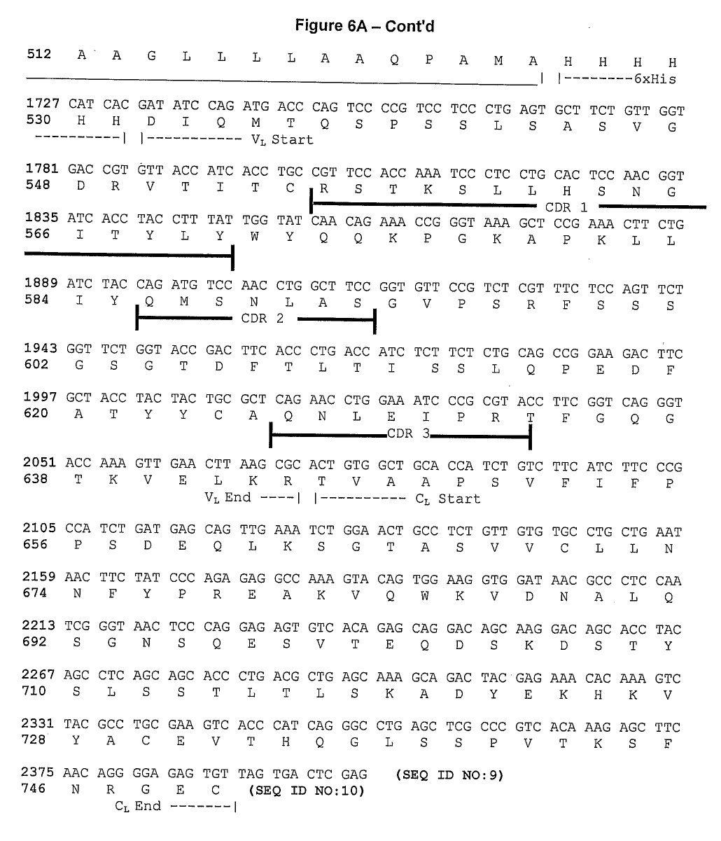

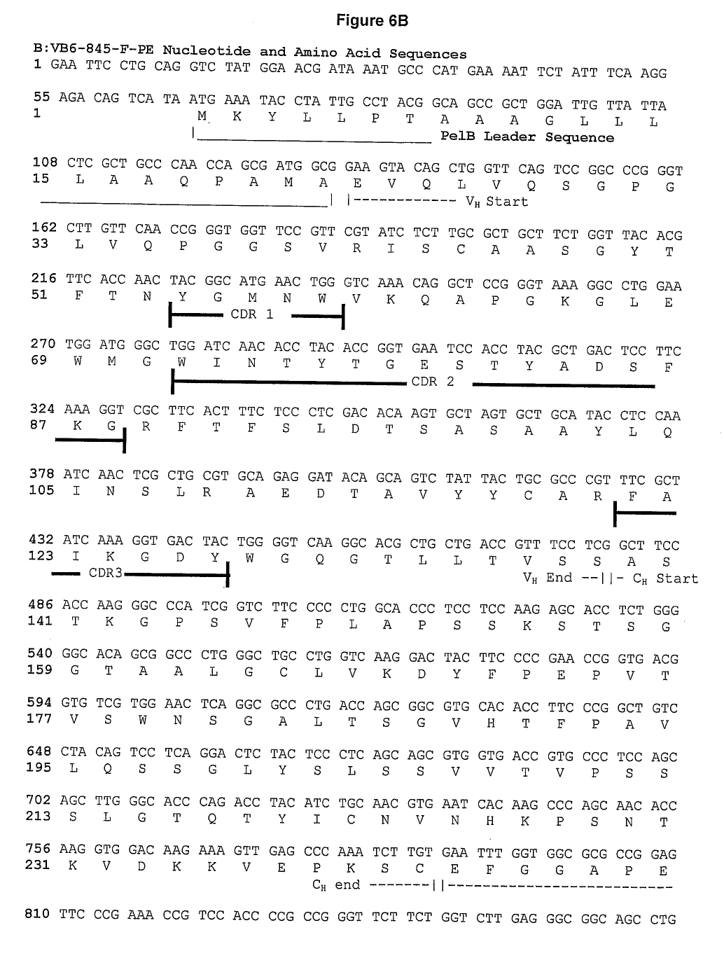

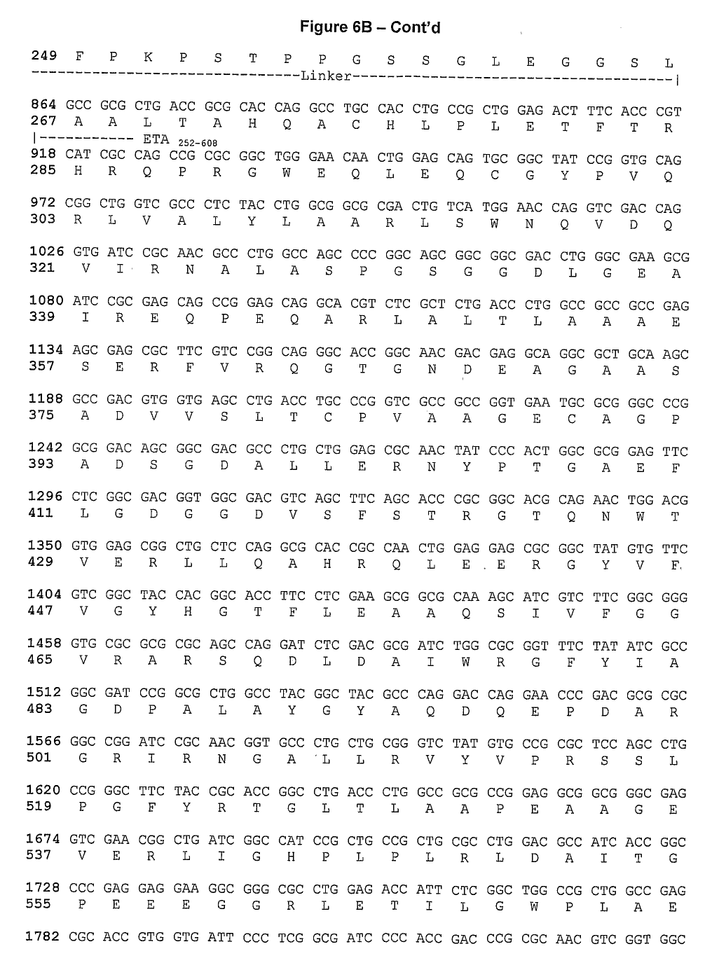

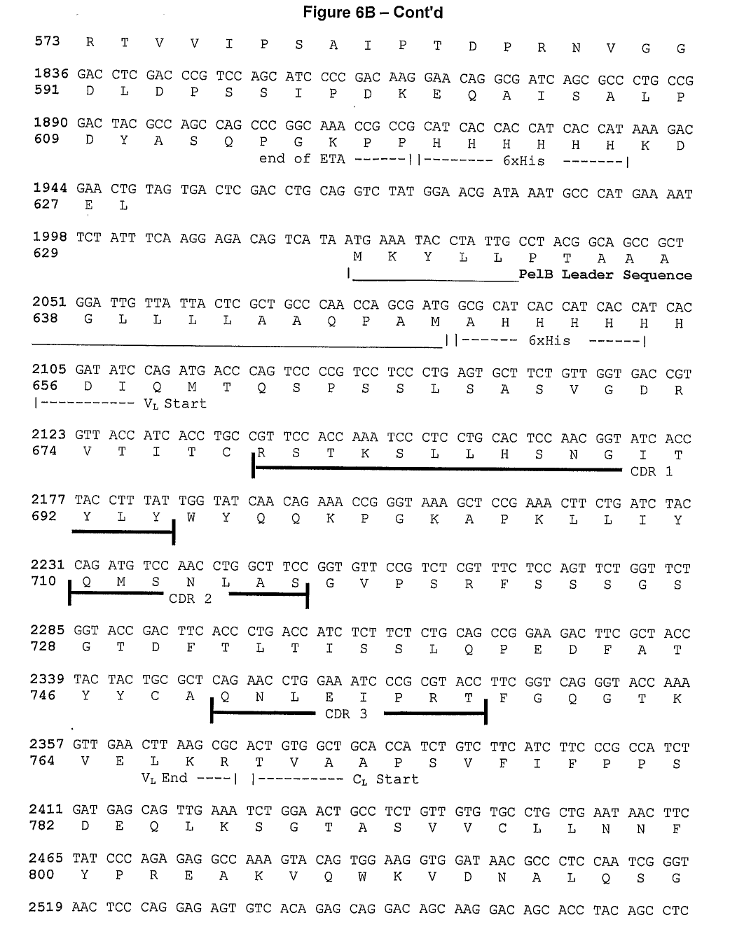

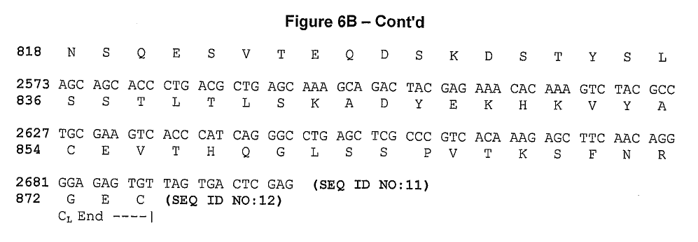

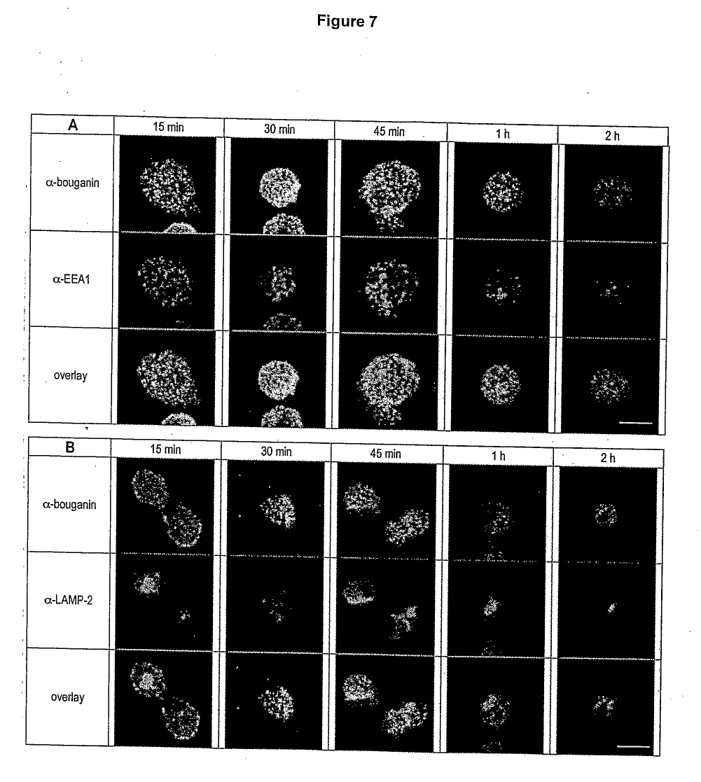

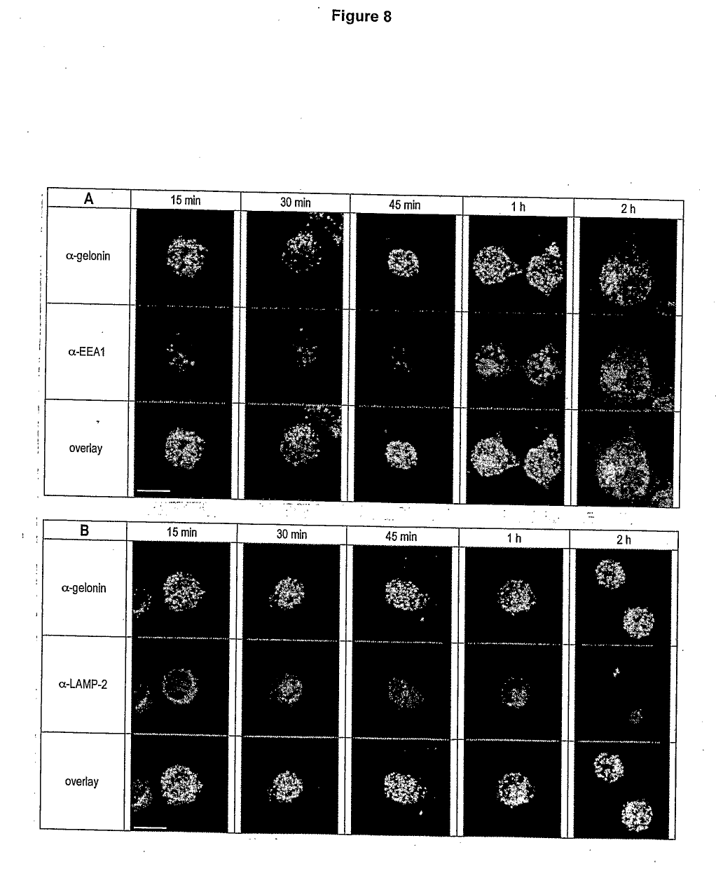

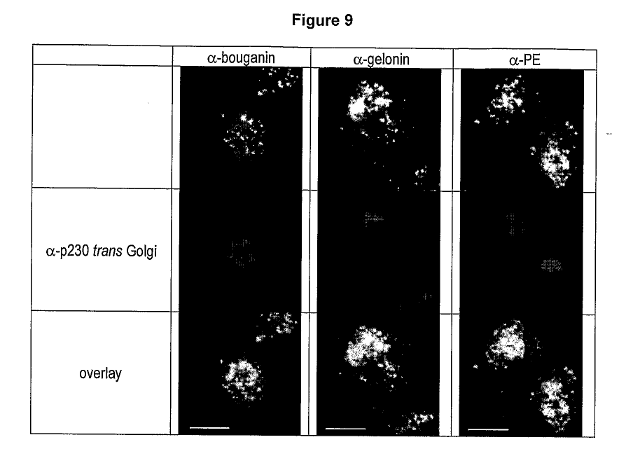

In order to determine de-bouganin trafficking, CAL-27 cells were treated with 5 nM of VB6-845-F-de-bouganin-Ala70 and stained with the anti-bouganin antibody after 15, 30, 45, 60, 120 or 180 minutes. Untreated cells served as a control for the specific staining of anti-bouganin, anti-gelonin and anti-PE antibodies. For time course experiments, 5 coverslips were used in a 6-well plate (Nunc™) to ensure the consistency of the assay. Images were captured on an Olympus IX-70 inverted confocal LASER microscope. The staining of treated cells revealed that de-bouganin was detected intracellularly after only 15 minutes. In addition, a punctate staining was observed suggesting localisation into intracellular compartments. To determine the nature of these compartments, the staining was performed simultaneously with a specific marker of either the endosome (EEA1), lysosome (LAMP-2) or the Golgi apparatus (p230 trans Golgi). The overlay of the green and red staining corresponding to VB6-845-F-de-bouganin-Ala70 and subcellular markers, respectively, showed that de-bouganin colocalised with the endosome after 15 minutes incubation, as indicated by a yellow coloration. The time course revealed that the colocalisation in the endosome was maximal after 45 minutes and decreased after 1 hour (FIG. 7A), as indicated by a decrease in intensity of the yellow coloration. In addition, VB6-845-F-de-bouganin-Ala70 also colocalised within the lysosome after 45 minutes incubation (FIG. 7B), also indicated by a yellow coloration. Similar staining pattern was observed with VB6-845-F-gelonin (SEQ ID NO: 9 and 10) (FIG. 6A) which is known to traffic via an endosome/lysosome pathway (FIGS. 8A and 8B, (Selbo et al., 2000). As a positive control for the colocalisation within the Golgi apparatus, CAL-27 cells were incubated with VB6-845-F-PE (pseudomonas exotoxin A) (SEQ ID NO: 11 and 12) (FIG. 6B) and stained with an anti-PE antibody. Indeed, it is well documented that PE traffics via the Golgi to the ER to reach the cytosol (Kreitman and Pastan, 1995). As shown in FIG. 9 a stronger colocalisation, shown by the yellow coloration, was observed between PE and the p230 trans Golgi marker.

### The Biological Activity of VB6-845-F-de-bouganin is Affected by a pH Increase in the Endosome and Lysosome Compartments

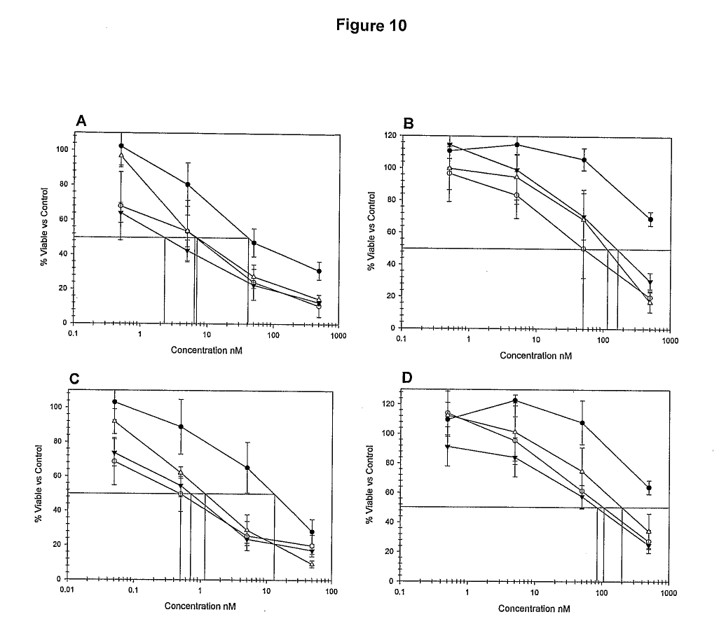

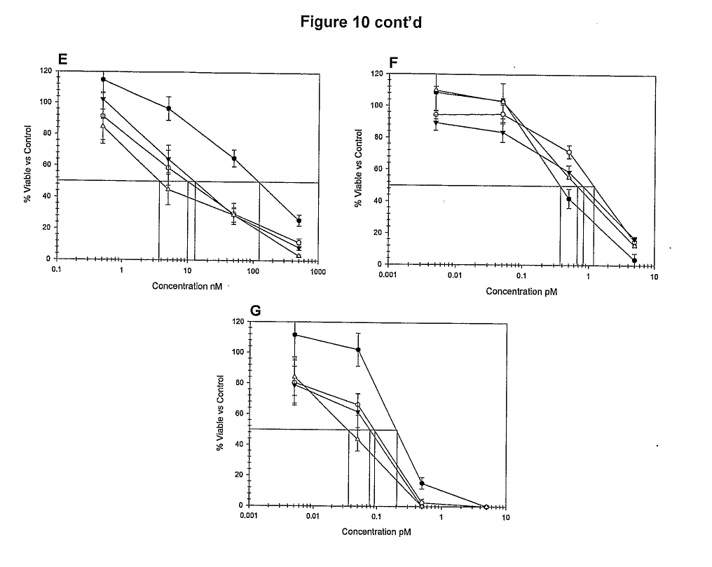

To link the biological activity of VB6-845-F-de-bouganin with endosome and lysosome compartments, cells were treated with VB6-845-F-de-bouganin at different concentrations (range from 0.5 nM to 500 nM) and with drugs that increase endosomal and lysosomal pH. The IC₅₀ was determined after 3 days on CAL-27 cells, a tumour Ep-CAM-positive cell line simultaneously treated with VB6-845-F-de-bouganin or other cytotoxins and with either 6.25 μM chloroquine, 10 mM NH₄Cl or 300 nM monensin (Table 1). The IC₅₀ of VB6-845-F-de-bouganin was reduced 5 to 9 fold with an average of 6.6 fold when cells were treated with chloroquine. VB6-845-F-de-bouganin cytotoxicity improved 10 to 20 fold in the presence of NH₄Cl (15.6 fold in average) and by 5.7 to 13.7 fold in the presence of monensin (FIG. 10A). Similarly, the presence of the drugs enhances de-bouganin cytotoxicity (FIG. 10B). Chloroquine or NH₄Cl treatment on CAL-27 treatment improves VB6-845-F-gelonin cytotoxicity 14.3 to 25 fold while monensin treatment enhances it by 5.6 to 10 fold (FIG. 10C). Gelonin showed an analogous pattern compared to its corresponding VB6-845-F-gelonin format (FIG. 10D). Saporin also showed a 10 fold improvement of its cytotoxicity in CAL-27 cells treated with chloroquine and NH₄Cl and even a 11.1 to 25 fold improvement for monensin (FIG. 10E). Cytotoxicity of VB6-845-F-PE was reduced 2.3 to 4 fold by an increase of endosomal and lysosomal pH by chloroquine and NH₄Cl and 1.1 to 1.8 fold by monensin (FIG. 10F) due to its dependence on pH to translocate through the membrane. PE alone is thought to use both endosomal/lysosomal and Golgi/ER pathway to trigger apoptosis (Smith et al., 2006). Ricin cytotoxicity was improved by drug treatments, although it was in a smaller range: 1.8 to 3.5 fold enhancement for chloroquine and NH₄Cl and 5 to 7 fold for monensin (FIG. 10G).

### VB6-845-F-de-bouganin Cytotoxicity is not Affected by an Irreversible Proteasome Inhibitor, Lactacystin

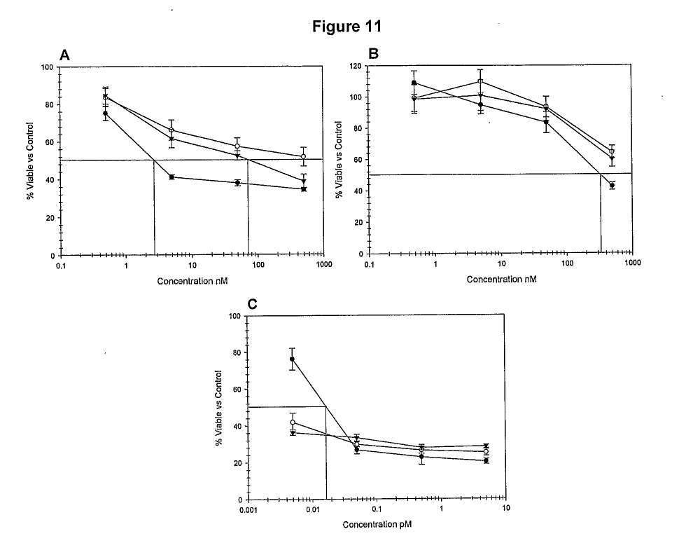

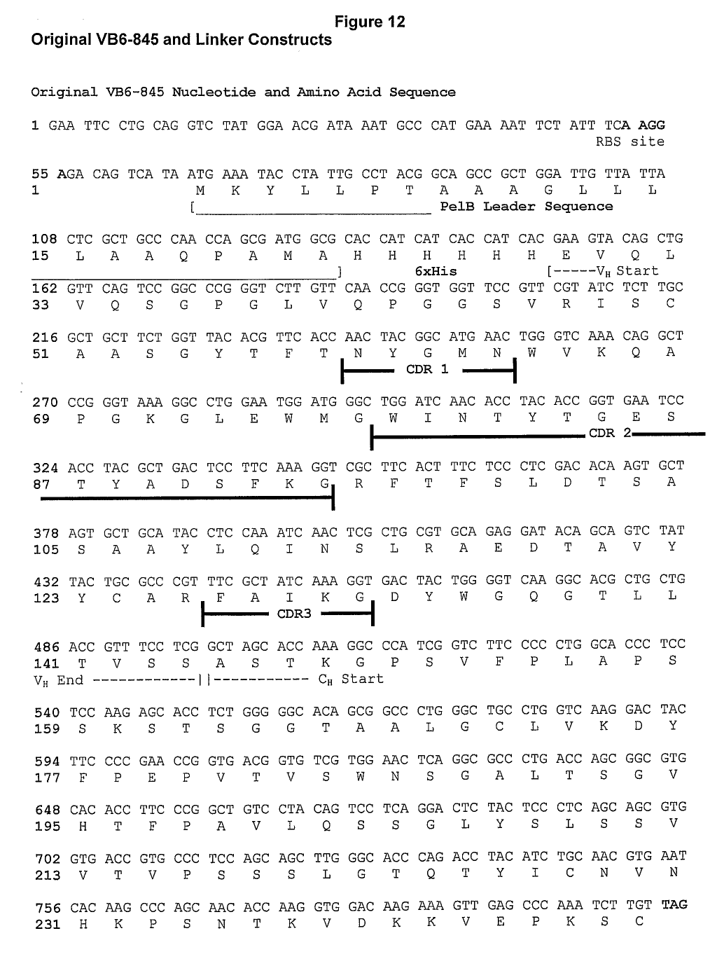

To study whether or not VB6-845-F-de-bouganin uses the ERAD pathway, cells were simultaneously treated with VB6-845-F-de-bouganin and lactacystin, an irreversible proteasome inhibitor. MCF7 viability in presence of lactacystin was greater than 80% in all our assays. After 3 days of treatment of 5 or 10 μM of lactacystin on MCF7 cells, the proteasome inhibitor reduces VB6-845-F-de-bouganin and de-bouganin cytotoxicity (FIGS. 11A and 11B). As PE uses the ERAD pathway (Teter and Holmes, 2002), it was used a positive control. Lactacystin inhibits proteasome degradation following the ERAD pathway, so more VB6-845-F-PE molecules reach the cytosol when co-administrated with lactacystin. VB6-845-F-PE cytotoxicity was improved by lactacystin (FIG. 11C).

### Example 3

### Immunotoxin Containing a Multi-Site Cleavable Linker

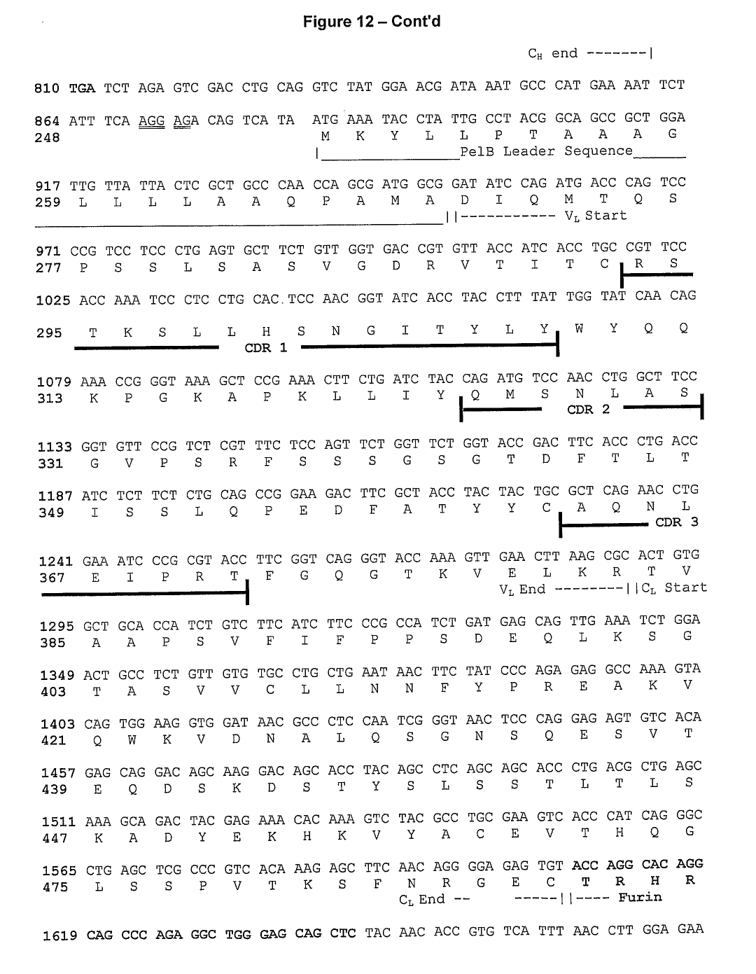

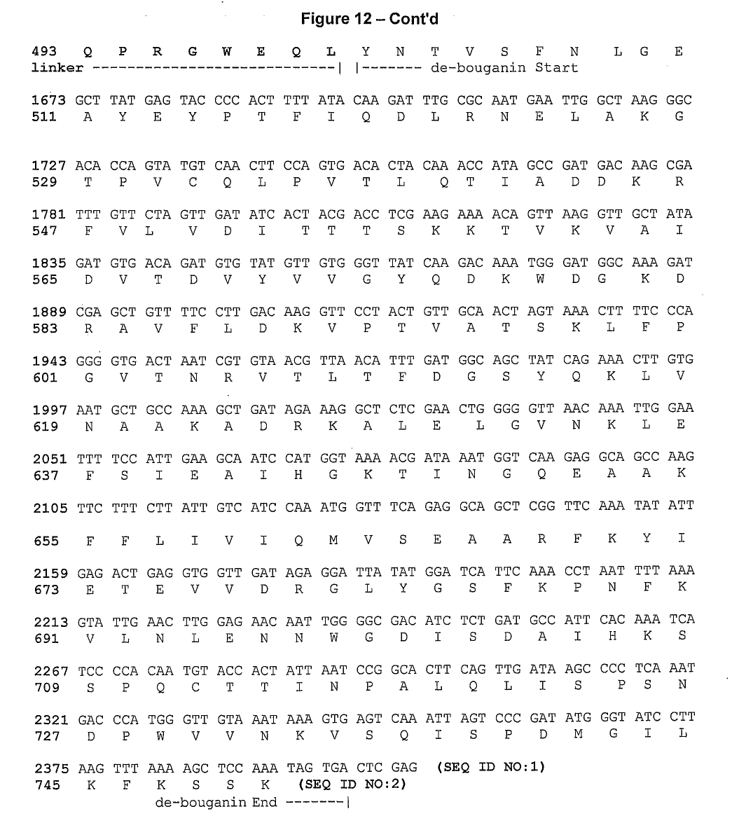

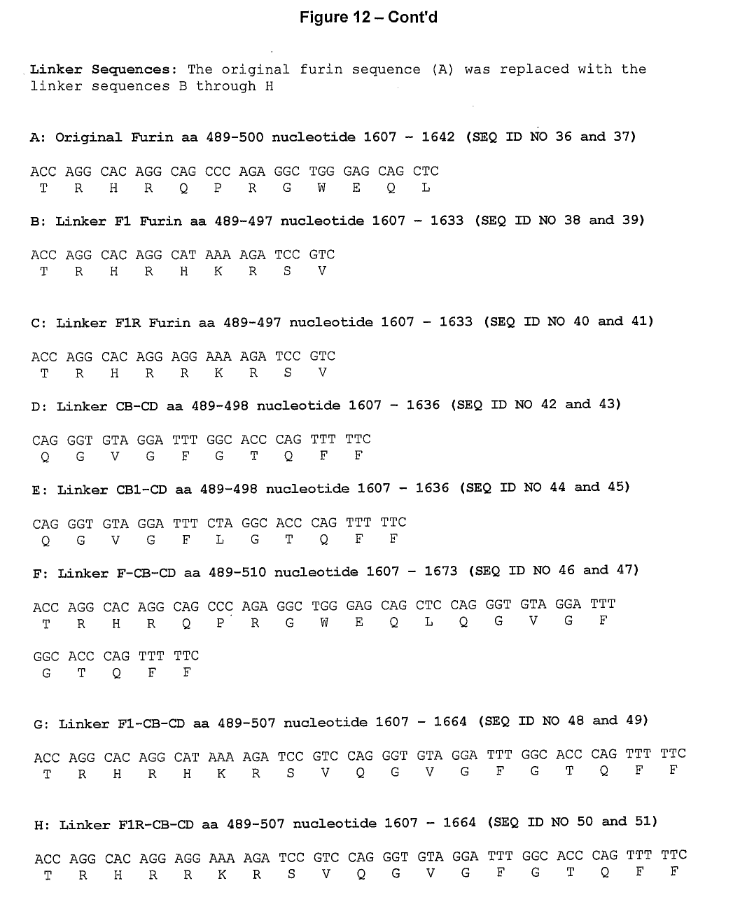

VB6-845-F-de-bouganin was re-engineered and tested with variants of furin specific site linkers (F1 and F1R) (FIG. 12, SEQ ID NOS:38 39 and 40-41 respectively and Table 2 SEQ ID NO: 16 and 19 respectively). The primers used for the engineering are shown in Table 2 (SEQ ID NO:14, and 17, 18 respectively). In addition to furin, tumor cells over-express other proteases such as the cathepsin family members, the urokinases and MMP proteases. Cathepsin D (an aspartic protease) and B (a cysteine protease) are normally found in the late endosome and lysosomes compartments of eukaryotic cells. High levels of cathepsin D and B have been associated with tumor progression and are useful in a pro-drug activation strategy. Using combinatorial amino acid libraries, optimal peptide substrates for both enzymes have been identified. Two cathepsin B and D specific linkers, CB-CD (Table 2 SEQ ID NO: 22 and FIG. 12 SEQ ID NO: 42 and 43) and CB1-CD (Table 2 SEQ ID NO 25 and FIG. 12 SEQ ID NO 44 and 45), were engineered using the primers listed in Table 2 (SEQ ID NO: 20, 21 and SEQ ID NO: 23, 24 respectively).

VB6-845-F-de-bouganin was also re-engineered and tested with protease sensitive sites of cathepsin D and B (CB-CD (SEQ ID NO:22 and SEQ ID NO 42 and 43, Table 2 and FIG. 12 and CB1-CD (SEQ ID NO:25 and SEQ ID NO: 44 and 45, Table 2 and FIG. 12). In order to test a potential additive or synergistic effect, the protease cleavage sites of cathepsin B and D were coupled to a furin specific site and the modified furin specific site linkers using primers listed in Table 2 (SEQ ID NO:26, 27 and 29,30 and 32,33) to create VB6-845-F-CB-CD-de-bouganin, (SEQ ID NO:28 (Table 2) and SEQ ID NO:46 and 47 (FIG. 12)) VB6-845-F1-CB-CD-de-bouganin (SEQ ID NO: 31 (Table 2) and SEQ ID NO: 48 and 49 (FIG. 12)) and VB6-845-F1R-CB-CD-de-bouganin (SEQ ID NO: 34 (Table 2) and SEQ ID NO: 50 and 51 (FIG. 12)).

Molecular Engineering of VB6-845-L-de-bouganin with Various Linkers (L)

The VB6-845-L-de-bouganin constructs were engineered by replacing the SacI-C_{L}-F-de-bouganin-HindIII fragment with SacI-C_{L}-L-de-bouganin containing various linkers (L) using the SacI and HindIII restriction sites. The SacI-C_{L}-L-de-bouganin fragments containing various linkers were assembled by the Splice Overlapping Extension Polymerase Chain Reaction method, SOE-PCR using VB6-845-F-de-bouganin DNA plasmids as templates and the primers listed in Table 2. The level of expression of the full-length VB6-845-de-bouganin with the variant furin linkers was similar to the wild-type. Therefore, only the F1R linker construct was purified and the biological activity was tested by flow cytometry and MTS assay.

### Biological Activity of the VB6-845-L-de-bouganin Linker Variants

EpCam Specific Cell Binding

Flow cytometry was used to demonstrate that the purified VB6-845-L-de-bouganin proteins retained their binding specificity using antigen-positive cell lines (NIH:OVCAR-3 and CAL 27) and an antigen-negative cell line (A-375). As expected, no binding was detected by flow cytometry after incubation with A-375. In contrast, a similar shift in median fluorescence was observed with all VB6-845-L-de-bouganin proteins incubated with CAL 27 and NIH:OVCAR-3 (Table 3). Since the Fab portion was not altered, the different linkers did not affect the binding activity of the VB6 proteins. The IC₅₀ was similar to the wild-type control despite the optimization of the furin cleavage site. The CB1-CD construct binding activity was similar to the wild-type and the IC₅₀ was in the nM range similar to the furin linker, especially for the NIH:OVCAR-3 cell line.

Cell Viabiltiy Assay

An MTS assay was utilized to determine the cytotoxicity of the variants using the antigen positive cell lines, NIH:OVCAR-3 and CAL 27, and antigen negative cell line A-375. A-375, CAL 27 and NIH:OVCAR-3 cell lines were incubated with an equimolar concentration of VB6-845-F-de-bouganin, VB6-845-CB1-CD-de-bouganin and VB6-845-F1R-de-bouganin ranging from 100 nM to 0.1 nM. After 5 days incubation, the calculated IC₅₀ of VB6-845-F-de-bouganin was 0.5 nM and 1.75±0.2 nM with NIH:OVCAR-3 and CAL 27, respectively (Table 3). For comparison, the IC₅₀ of VB6-845-F1R-de-bouganin with NIH:OVCAR-3 and CAL 27 was 0.6 and 1.7±0.8 nM, respectively. An IC₅₀ of 2.5 nM and 12 nM was obtained with VB6-845-CB1-CD-de-bouganin when incubated with NIH:OVCAR-3 and CAL 27. In contrast, no cytotoxicity was observed with A-375 cells.

### Example 4

### Immunotoxin with Optimized Nucleotide Sequence Containing Multi-Site Cleavable Linker

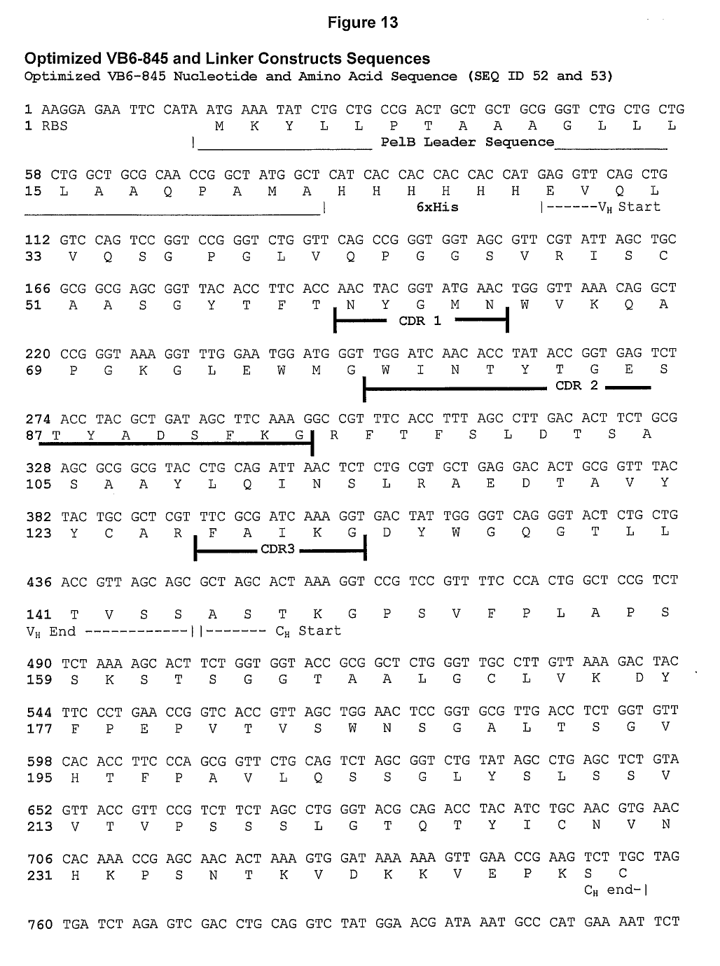

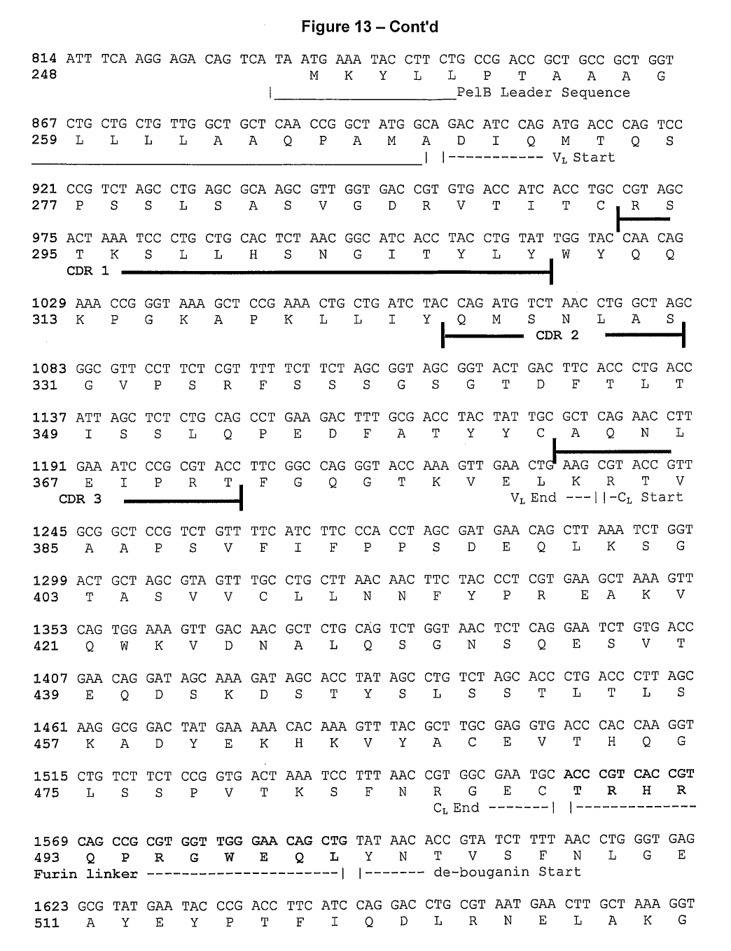

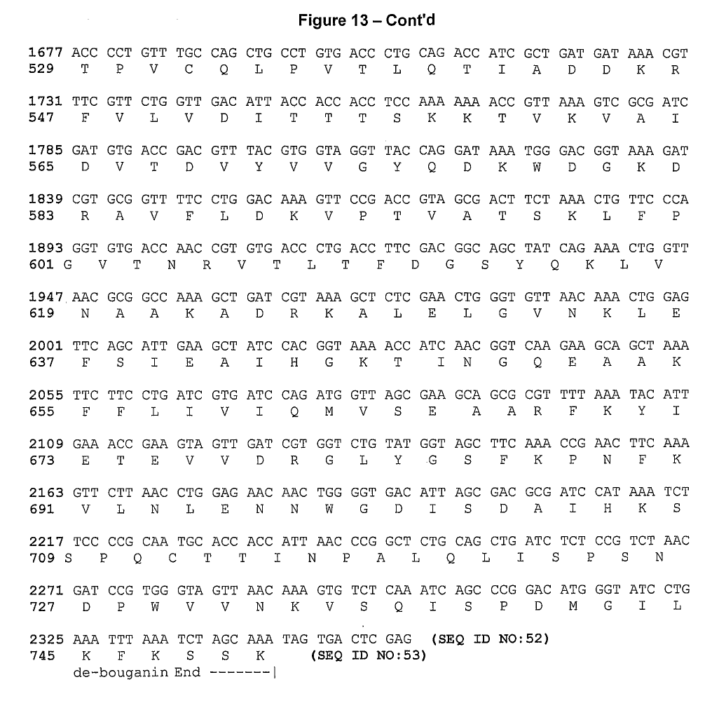

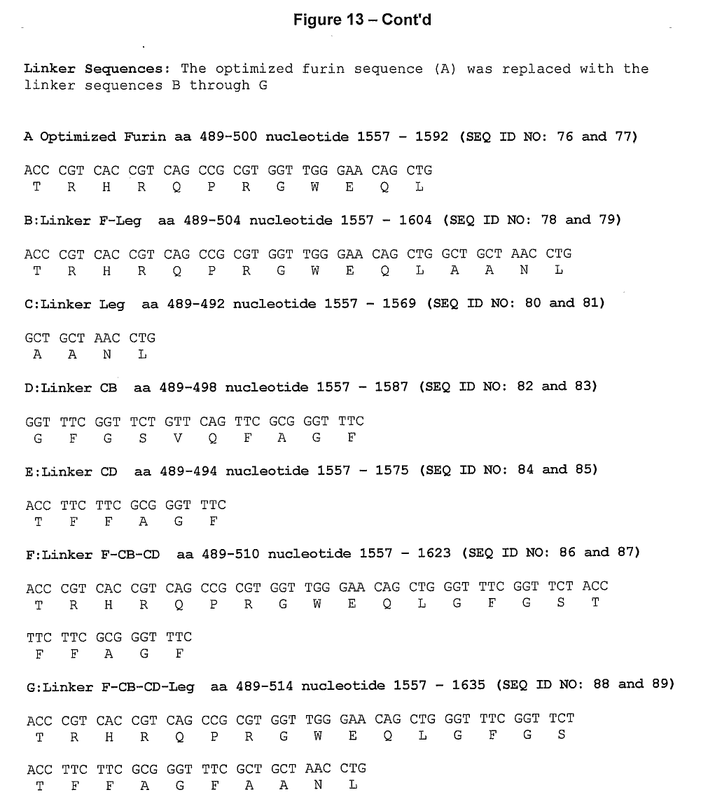

Different protease linker variants of VB6-845-de-bouganin containing multiple proteolytic sites were generated. The optimization of VB6-845-F-de-bouganin nucleotide sequence (SEQ ID NO: 52) has led to a 10 times increased expression level compared to the non-optimized sequence as described in a co-pending application (U.S. Ser. No. 60/912,732). To improve VB6-845-F-de-bouganin cytotoxicity, different variant linkers are inserted into the nucleotide optimized construct of VB6-845-F-de-bouganin (FIG. 13). The linkers will contain the furin site (F) and proteolytic site of proteases which are localized in the endosome/lysosome compartments. Therefore, proteolytic sensitive sites for cathepsin D and B (CD and CB) (GFGSTFFAGF) (SEQ ID NO: 54) which has been validated in vivo are added to the furin linker (DeNardo et al., 2003). In addition, the proteolytic site of the asparaginyl legumain protease (Leg) (AANL) (SEQ ID NO: 55) which is over-expressed in many solid tumors is evaluated as well (Liu et al., 2003). The expression level and IC₅₀ of each construct is determined and compared to that of nucleotide sequence optimized VB6-845-F-de-bouganin.

### Engineering of Nucleotide Sequence Optimized VB6-845-F-Leg-de-bouganin, VB6-845-Leg-de-bouganin, VB6-845-F-CB-CD-de-bouganin, VB6-845-CB-de-bouganin and VB6-845-CD-de-bouganin

The 5′ C_{L}-F-Leg and 3′ F-Leg-dB fragments were obtained by PCR using VB6-845/pING3302 as a template with the pairs of primers 5′ C_{L}-Kappa-CODA-StyI (SEQ ID NO: 56) and 3′ F-Leg (SEQ ID NO: 59) and primers 5′ F-Leg (SEQ ID NO: 58) and 3′Boug-CODA-XhoI (SEQ ID NO: 57) respectively (Table 4). The cycling program used had 20 cycles of 94° C. for 1 min, 62° C. for 1 min, 72° C. for 1 min and then 72° C. for 10 min.

The obtained fragments were linked together by splice overlapping extension PCR (SOE-PCR) method using primers 5′ C_{L}-Kappa-CODA-StyI (SEQ ID NO: 56) and 3′Boug-CODA-XhoI (SEQ ID NO: 57).

The resulting PCR insert C_{L}-F-Leg-dB was purified with QIAquick® Gel Extraction Kit (QIAGEN), cloned into pCR®2.1-TOPO® Vector from TOPO TA Cloning® Kit (1:5 ratio) and transformed into chemically competent E. coli 10F cells (selection on Luria-Bertani [LB] agar kanamycin plate, 100 μg/mL). Two plasmids containing the insert were extracted using QIAprep® Spin Miniprep kit (QIAGEN) and sequenced with CEQ™ 8000 Genetic Analysis System (Beckman Coulter) as described below. A C_{L}-F-Leg-dB/pCR®2.1 clone with verified sequence was digested first with EcoRI (Invitrogen™) and then with StyI and XhoI (New England Biolabs® Inc and Invitrogen™, respectively). The purified product of these digestions was ligated with VB6-845/pSP73 plasmid pre-digested with StyI and XhoI to engineer VB6-845-F-Leg/pSP73 (ligation reaction: Insert: Vector Molar Ratio of 2:1 with a total DNA of 0.5 μg; 4 μL of Ligase Reaction Buffer; 2 units of T4 DNA Ligase (1 unit/μL) Invitrogen™; in 20 μL). The ligation reaction was transformed into chemically competent E. coli 10F cells and the transformants were selected on LB agar plates in the presence of 100 μg/mL ampicillin. To check this ligation efficiency a PCR-screening of the colonies, using primers 5′ C_{L}-Kappa-CODA-StyI (SEQ ID NO: 56) and 3′Boug-CODA-XhoI, (SEQ ID NO: 57) was done and its product, as a VB6-845 control, was digested with Eco01091 and separated on a 10% Acrylamid gel (1.44 M Acrylamid—80 mM Tris Acetate—2 mM EDTA—3.04 mM Persulfate Ammonium—3 mM TEMED—34.4 M H₂O). Plasmid extracted from two positive clones was digested with ScaI (Invitrogen™) then with EcoRI and XhoI. The VB6-845-F-Leg-de-bouganin (SEQ ID NO 52 and 78) and 2.4 Kb fragment was then ligated with the pING3302 vector predigested with EcoRI and XhoI. The ligation reaction was transformed into chemically competent E. coli 10F cells and the transformants were selected on LB agar plates in the presence of 25 μg/mL tetracycline. A pING3302 vector containing the sequenced VB6-845-F-Leg-de-bouganin insert was transformed into E. coli E10⁴ cells. Using the same template and method, the following constructs VB6-845-F-CB-CD-de-bouganin (SEQ ID 52, 53 and 86, 87), VB6-845-F-CB-CD-Leg-de-bouganin (SEQ ID NOS: 52, 53 and 88, 89), VB6-845-Leg-de-bouganin (SEQ ID NOS: 52, 53 and 80, 81), VB6-845-CB-de-bouganin (SEQ ID NOS: 52, 53 and 82, 83), and VB6-845-CD-de-bouganin (SEQ ID NOS:52, 53 and 84, 85), were engineered using primers 5′ C_{L}-Kappa-CODA-StyI (SEQ ID NO: 56) and 3′Boug-CODA-XhoI (SEQ ID NO: 57) and primers listed in Table 4 (SEQ ID NO: 58 to 69). FIG. 13 shows the full nucleotide and amino acid sequence of the VB6-845-L-de-bouganin constructs and linkers respectively (SEQ ID NOS:52, 53 and 76 to 89).

Growth, Expression and Analysis of the Culture Supernatant of VB6-845-F-Leg-de-bouganin, VB6-845-Leg-de-bouganin, VB6-845-F-CB-CB-Leg-de-bouganin, VB6-845-F-CB-CD-de-bouganin, VB6-845-CB-de-bouganin and VB6-845-CD-de-bouganin Clones by Western Blot

To ensure that the various engineered VB6-845-L-de-bouganin linkers did not affect the quality and quantity of expressed soluble material, supernatant from each expressed clone were analyzed by Western blot and full-length protein quantified by ELISA.

Two clones of engineered constructs of VB6-845-L-de-bouganin were propagated in 30 mL of TB media (1% innoculum) in a 250 mL shake flask at 37° C., shaken at 225 rpm for approximately 5 hours until the optical density (O.D. 600 nm) reaches 2. At this time, the culture was induced with a final concentration of 0.1% L-(+) arabinose for 16 hours and incubated at 25° C. Subsequently, the supernatant was collected by centrifugation at 14000 rpm for 5 minutes and analyzed by Western blot using an anti-human kappa light chain under reducing and non-reducing conditions to confirm the presence and size of the immunotoxin A. Under non reducing conditions, the induced supernatant (16 μL) was separated on precast 10% sodium dodecyl sulfate polyacrylamid gel electrophoresis (SDS-PAGE) using NuPAGE® SDS MOPS Running Buffer (Invitrogen™) and transferred to a nitrocellulose membrane with NuPAGE® Transfer Buffer (Invitrogen™) complemented with 20% methanol. The membrane was blocked with 3% BSA in Tris-Buffered Saline (TBS, 50 mM Tris base—150 mM NaCl—pH 7.4) for 1 h at Room Temperature (RT), washed twice in TBS complemented with 0.05% Tween®20 (TBS-T) and incubated for 2 h at RT with a goat anti-human kappa light chain peroxidase conjugate antibody (Sigma®) diluted 1/1000 in TBS-T. Then, the membrane was washed four times for 5 min each in TBS-T and the binding of the goat anti-human kappa light chain peroxidase conjugate antibody to VB6-845-L-de-bouganin proteins was revealed by 3,3′-diaminobenzidine tetrahydrochloride (DAB/Metal concentrate and its buffer) from Pierce according to the manufacturer's instructions. The induced VB6-845/pING3302 supernatant was used as a positive control.

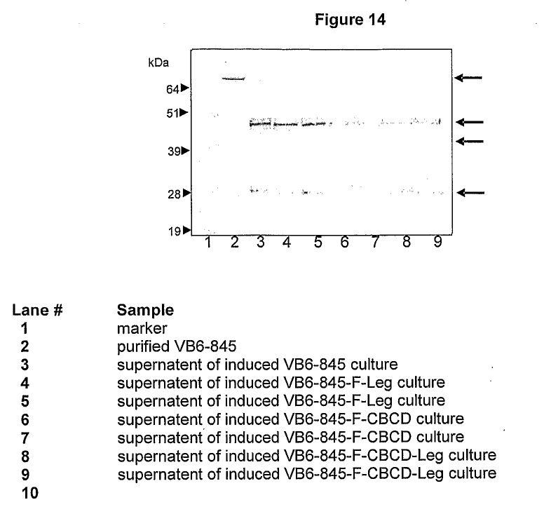

Western blot analysis of the VB6-845-L-de-bouganin linker variants induced supernatant show a lower level of expression than nucleotide sequence optimized VB6-845-F-de-bouganin (FIG. 14).

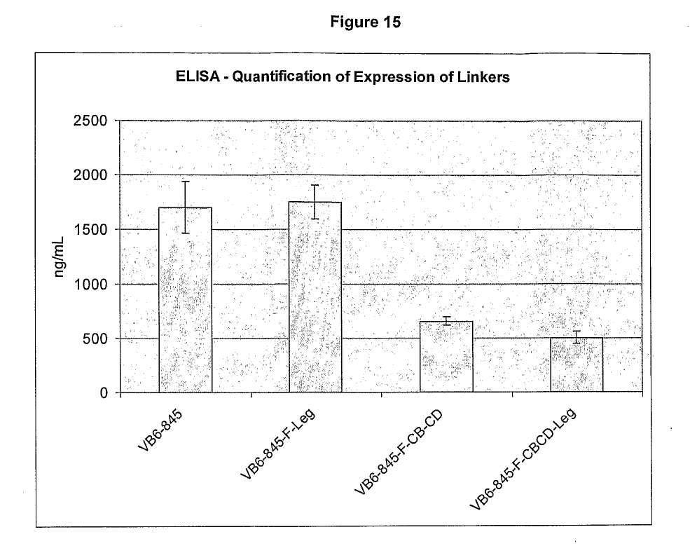

To confirm this data, quantification by ELISA was performed. A 96-well Immunolon® 2 High Binding Flat Bottom Microtiter® Plate (DYNEX Technologies) was coated with 100 μL/well of rabbit anti-bouganin diluted at 10 μg/mL in coating buffer (35 mM sodium bicarbonate—15 mM sodium carbonate—pH 9.6) and incubated overnight at 4° C. After washing three times with 0.5% Tween®20 in Phosphate Buffer Saline (PBS, 136 mM NaCl—8 mM Na₂HPO₄—1.47 mM KH₂PO₄—2.68 mM KCl—pH 7.0), plates were blocked with 200 μL/well of 1% BSA in PBS for 1 h at RT. Diluted VB6-845-F-CB-CD-de-bouganin supernatants starting from 1/300 to 1/2400 in dilution buffer (0.5% Tween®20—0.5% BSA in PBS) were incubated for 2 h at RT. The standard curve was obtained with purified VB6-845-F-de-bouganin protein starting at 50 ng/mL to 0.78 ng/mL. After three washes in 0.5% Tween®20 in PBS, 100 μL/well of a mouse anti-human IgG Fd monoclonal antibody solution (1/4000) was added and incubated at RT for 1 h. After three washes, 100 μL/well of anti-mouse IgG (H+L) antibody biotin conjugated (1/2000) was added and incubated 1 h. Following three washes, 100 μL/well of substrate solution with Horse Radish Peroxidase (HRP) streptavidin conjugated (Pierce) was added per well and the plate was incubated 30 min at RT. After a final wash, 100 μL/well of HRP substrate was added per well and the reaction was stopped with 100 μL/well of a 1 M phosphoric acid solution after 2 min incubation. Absorbance was measured at a wavelength of 450 nm, VB6-845-F-de-bouganin and VB6-845-F-Leg-de-bouganin levels of expression were around 1.7 μg/mL. A significant decrease of 2.1 and 3.8 fold was observed with VB6-845-F-CB-CD-de-bouganin and VB6-845-F-CB-CD-Leg-de-bouganin supernatant, respectively (FIG. 15). Due to poor expression, VB6-845-F-CB-CD-Leg-de-bouganin was not tested further.

### Biological Activity of VB6-845-L-de-bouganin Linker Variants

Flow cytometry was used to demonstrate that the purified VB6-845-L-de-bouganin linker variants retain their binding specificity using antigen-positive cell lines (OVCAR-3 and Cal-27) and an antigen-negative cell line (A-375). Binding was detected using a rabbit anti-bouganin antibody and compared to VB6-845-F-de-bouganin. EpCAM positive tumor cells were resuspended at 0.3×10⁶ per mL in flow cytometry buffer (10% foetal bovine serum in PBS) and transferred into 5 mL polystyrene round-bottom tubes (BD Falcon™). Cells were washed in flow cytometry buffer and incubated with 1 μg/mL of VB6-845-F-de-bouganin control and the test VB6-845-L-de-bouganin linker variants on ice for 2 h with several manual mixes. After being washed twice with the same buffer, cells were incubated for 1 h on ice with a polyclonal rabbit anti-bouganin, obtained from the serum of an immunized rabbit (purified by chromatography affinity using a protein A column) used at 1/100 dilution in flow cytometry buffer. After two washes, a third step reagent, goat anti-rabbit FITC conjugate immunoglobulin (The Binding Site) used at 1/100 dilution in flow cytometry buffer was added to detect the bound rabbit anti-bouganin to VB6-845-F-de-bouganin control, and the VB6-845-L-de-bouganin linker variants. After 30 min incubation on ice, cells were washed. Propidium iodide (Molecular Probes™) was then added to reveal non-viable cells. The cells were then analyzed on a BD FACSCalibur™ cytometer equipped with air-cooled argon-ion LASER, Light Amplification Stimulated Emission of Radiation, (Becton, Dickinson and Company). A minimum of 10000 events were analyzed using BD CellQuest™ software (BD Biosciences). Using the forward scatter (FSC) detector and the intensity of the red fluorescence, FL, (Propidium iodide—FL2 detection), an area containing live cells was defined as R1. For fluorescence analysis, the intensity of the green fluorescence (FITC—FL1 detection) was obtained from the R1 area. Voltage of FL1 was defined to place the negative control cells below 10¹, containing at least 95% of these cells. Markers M1, between 10° and 10¹, and M2, between 10¹ and 10⁴, were determined. M2 values define the percentage of the tested VB6-845-L-de-bouganin bound to EpCAM-positive cells.

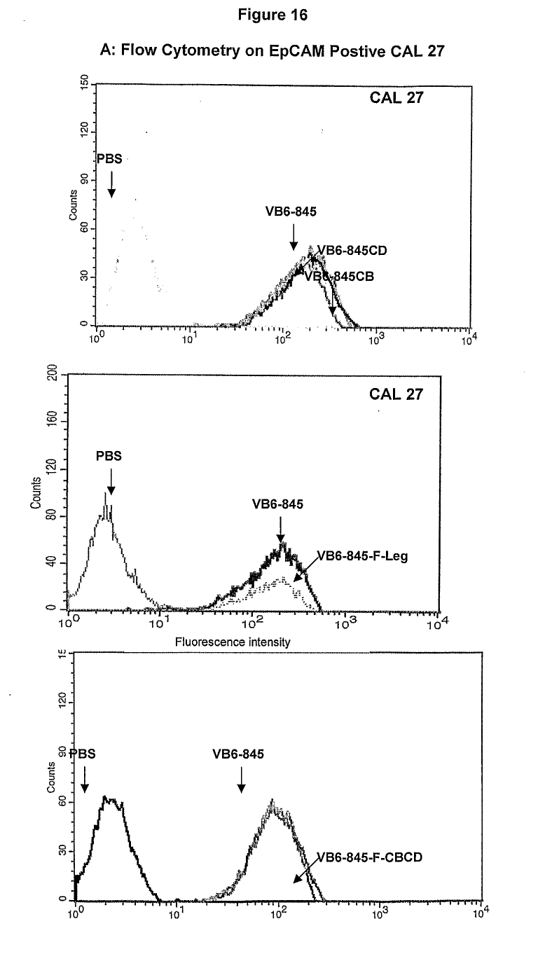

As expected, no binding was detected by flow cytometry after incubation with A-375. In contrast, a similar shift in median fluorescence was observed with all VB6-845-L-de-bouganin linker variant proteins incubated with CAL-27, (FIG. 16).

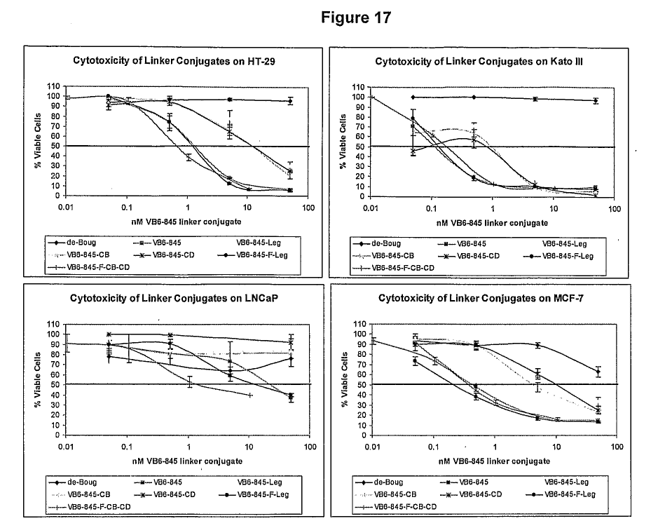

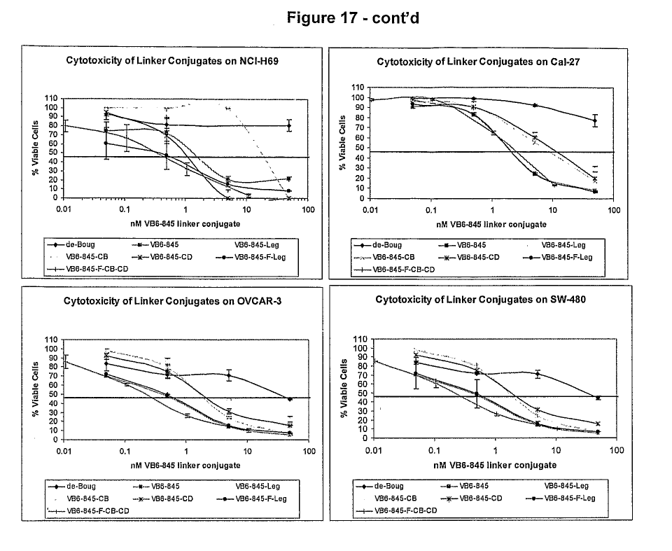

An MTS assay was used to determine the IC₅₀ values of the variants using a variety of EpCAM-positive cell lines from different indications such as Cal-27 (head and neck), HT29 (colon), Kato III (stomach), LNCaP (prostate), MCF-7 (breast), OVCAR-3 (ovarian) and SW-480 (colon). The IC₅₀ of the variants was determined and compared to VB6-845-F-de-bouganin. Cells seeded at 1000 per well in flat bottomed 96-well plates (Nunc™) were incubated with VB6-845-Leg-de-bouganin, VB6-845-F-Leg-de-bouganin, VB6-845-F-CB-CD-de-bouganin, VB6-845-CB-de-bouganin or VB6-845-CD-de-bouganin using 10 fold serial dilutions in triplicate wells. VB6-845-F-de-bouganin and de-bouganin were used as control. The plates were then incubated at 37° C. in a 5% CO₂-supplemented atmosphere for 5 days. Cell viability was assessed by reduction of the tetrazolium salt (MTS) to the formazan product by dehydrogenase enzymes found in metabolically active cells (CellTiter 96® Aqueous Non-Radioactive Cell Proliferation Assay, Promega). The data was expressed as a percentage of absorbance at 490 nm observed in untreated cells. The observed effect of the VB6-845-de-bougain linker variants differed, VB6-845-F-de-bouganin was cytotoxic to each cell line except LNCaP and was more cytotoxic in all the cell lines tested than the any of the constructs with the cathepsin or legumain sites alone. The IC50 for VB6-845-F-CB-CD-de-bouganin construct was not significantly different than that of VB6-845-F-de-bouganin in 3 of the 8 cell lines tested (CAL-27, Kato III and MCF-7). For the other 5 of 8 cell lines (LNCaP, HT-29, NCI-H69, NIH-OVCAR-3 and SW-480) the VB6-845-F-CB-CD-de-bouganin construct was more toxic than VB6-845-F-de-bouganin by at least 1.3 (SW-480) to over 6.2 fold. (LNCaP) (FIG. 17 and Table 5). The construct containing both the furin and the legumain sites was more cytotoxic than that containing just the furin site in 4 of the 8 cell lines tested (LNCaP, MCF-7, NCI-H69 and SW480) by at least 1.1 (LNCaP) to almost 5 fold (NCI-H69) (FIG. 17 and Table 5). It should be noted that linkers containing a furin site with either cathepsin or leguman sites added showed significantly improved cytotoxicity on both LNCaP, (prostate cancer cell line) and on NCI-H69 (small cell lung cancer).

The addition of the legumain or cathepsin B and D sites to the furin linkers have an additive or synergistic cytotoxic effect and broaden the range of efficacy for cancer types. Addition of linkers containing protease sensitive sites specific for the orgnanelles through which a toxin traffics or the region of the cell targeted improves the cytotoxic effect.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

TABLE 1
Effect of alkalinisation of endosomal and lysosomal pH
on VB6-845-F-immunotoxins cytotoxicity on CAL-27 cells.

| | Chloroquine | NH₄Cl | Monensin |
|---|---|---|---|
| VB6-845-F-de- | 6.6 ± 2.1 | 15.6 ± 5.1 | 9.8 ± 4.0 |
| bouganin | | | |
| De-bouganin | N/A | N/A | N/A |
| VB6-845-F- | 22.5 ± 3.5 | 19.7 ± 7.6 | 7.8 ± 3.1 |
| gelonin | | | |
| Gelonin | N/A | N/A | N/A |
| Saporin | 10.0 ± 0.0 | 10.0 ± 0.0 | 18.1 ± 9.8 |
| Ricin | 2.0 ± 0.3 | 3.0 ± 0.7 | 6.0 ± 1.4 |
| VB6-845-F-PE | −3.1 ± 0.7 | −2.8 ± 0.7 | −1.5 ± 0.5 |

Means and standard deviations for two to four independent experiments measuring fold-change in IC₅₀ of different cytotoxins in the presence of either 6.25 μM chloroquine, 10 mM NH₄Cl or 300 nM monensin on CAL-27 cells. N/A: not applicable for de-bouganin and gelonin, the IC₅₀ of the non treated cells are higher than 500 nM and therefore, no IC₅₀ and fold improvement can be determined.

TABLE 2
Primers used to engineer the various linkers (L) for
the non-optimized nucleotide sequence VB6-845-de-
bouganin and corresponding amino acid sequence

| | | Amino acid Sequence |
|---|---|---|
| Name | Nucleotidic sequence of the primers | of Linker |
| 5′ C_{L}-SacI | 1-5′ TAC GCC TGC GAA GTC ACC CAT CAG GGC | |
| | CTG AGC TCG CCC GTC | |
| | (SEQ ID NO: 13) | |
| Linker F1 | 2-5′: ACC AGG CAC AGG CAT AAA AGA TCC | TRHRHKR↓SV |
| | GTC TAC | (SEQ ID NO: 16) |
| | (SEQ ID NO: 14) | |
| | 3-3′: GTT GTA GAC GGA TCT TTT ATG CCT | |
| | GTG CCT | |
| | (SEQ ID NO: 15) | |
| Linker F1R | 2-5′: ACC AGG CAC AGG AGG AAA AGA TCC | TRHRRKR↓SV |
| | GTC TAC AAC ACC GTG TCA TTT AAC CTT GGA | (SEQ ID NO: 19) |
| | GAA | |
| | (SEQ ID NO: 17) | |
| | 3-3′: GAC GGA TCT TTT CCT CCT GTG CCT | |
| | GGT ACA CTC TCC CCT GTT GAA | |
| | (SEQ ID NO: 18) | |
| Linker CB-CD | 2-5′: CAG GGT GTA GGA TTT GGC ACC CAG | QGVGF↓GTQF↓F |
| | TTT TTC TAC AAC ACC GTG TCA TTT AAC CTT | (SEQ ID NO: 22) |
| | GGA | |
| | (SEQ ID NO: 20) | |
| | 3-3′: CTG GGT GCC AAA TCC TAC ACC CTG | |
| | ACA CTC TCC CCT GTT GAA GCT CTT | |
| | (SEQ ID NO: 21) | |
| Linker CB1- | 2-5′: GGA TTT CTA GGC ACC CAG TTT TTC TAC | QGVGF↓LGTQF↓F |
| CD | AAC ACC | (SEQ ID NO: 25) |
| | (SEQ ID NO: 23) | |
| | 3-3′: GAA AAA CTG GGT GCC TAG AAA TCC | |
| | TAC ACC | |
| | (SEQ ID NO: 24) | |
| Linker F-CB- | 2-5′: CAG GGT GTA GGA TTT GGC ACC CAG | TRHRQPR↓GWEQL |
| CD | TTT TTC TAC AAC ACC GTG TCA TTT AAC CTT | QGVGF↓GTQF↓F |
| | GGA | (SEQ ID NO: 28) |
| | (SEQ ID NO: 26) | |
| | 3-3′: CTG GGT GCC AAA TCC TAC ACC CTG | |
| | GAG CTG CTC CCA GCC TCT GGG CTG CCT | |
| | GTG CCT GGT | |
| | (SEQ ID NO: 27) | |
| Linker F1-CB- | 2-5′: CAG GGT GTA GGA TTT GGC ACC CAG | TRHRHKR↓SV |
| CD | TTT TTC TAC AAC ACC GTG TCA TTT AAC CTT | QGVGF↓GTQF↓F |
| | GGA | (SEQ ID NO: 31) |
| | (SEQ ID NO: 29) | |
| | 3-3′: CTG GGT GCC AAA TCC CAT ACC CTG | |
| | GAC GGA TCT TTT ATG CCT GTG CCT GGT | |
| | ACA CTC TCC CCT GTT GAA | |
| | (SEQ ID NO: 30) | |
| Linker F1R- | 2-5′: CAG GGT GTA GGA TTT GGC ACC CAG | TRHRRKR↓SV |
| CB-CD | TTT TTC TAC AAC ACC GTG TCA TTT AAC CTT | QGVGF↓GTQF↓F |
| | GGA | (SEQ ID NO: 34) |
| | (SEQ ID NO: 32) | |
| | 3-3′: CTG GGT GCC AAA TCC TAC ACC CTG | |
| | GAC GGA TCT TTT CCT CCT GTG CCT GGT | |
| | ACA CTC TCC CCT GTT GAA | |
| | (SEQ ID NO: 33) | |
| 3′de | 4-5′ CTC GAG CTA CTA TTT GGA GCT TTT AAA | |
| bouganin- | CTT AAG GAT ACC | |
| stop-XhoI: | (SEQ ID NO: 35) | |

F: Furin, CB-CD: Cathepsin B and D. The arrow indicates the cleavage site.

TABLE 3
Comparison of the Median Fold (MF) increase and
IC₅₀ (nM) of VB6-845-F-de-bouganin, VB6-845-F1R-
de-bouganin and VB6-845-CB1-CD-de-bouganin

| | CAL 27 | | OVCAR-3 | |
|---|---|---|---|---|
| | MF | IC₅₀ (nM) | MF | IC₅₀ (nM) |
| VB6-845_{L}-F- | 63.6 15.2 | 1.75 ± 0.25 | 130.5 ± 9.5 | 0.5 ± 0 |
| de-bouganin | | | | |
| VB6-845-F1R- | 55 ± 11 | 1.7 ± 0.8 | 112 ± 7 | 0.6 ± 0 |
| de-bouganin | | | | |
| VB6-845-CB1-CD- | 68* | 12* | 110* | 2.5* |
| de-bouganin | | | | |

One representative data, otherwise data is from two independent experiments.

TABLE 4
Primers used to engineer the various linkers (L) for
the Nucleotide Sequence Optimized VB6-845-F-de-bouganin
along with the corresponding amino acid sequence.

| | | Amino Acid |
|---|---|---|
| | | Sequence of |
| Name | Primer sequence | Linker |
| 5′ C_{L}-Kappa- | 5′TACGCTTGCGAGGTGACCCACCAAGGTCTG | N/A |
| CODA-Styl | (SEQ ID NO 56) | |
| 3′Boug- | 5′GCGCTCGAGTCACTATTTGCTAGATTTAAATTTCAG | N/A |
| CODA-XhoI | (SEQ ID NO 57) | |
| F-Leg | 1 5′:TGGGAACAGCTGGCTGCTAACCTGTATAACACCGTATCTTTT | TRHRQPR↓G |
| | AACCTG | WEQLAAN↓L |
| | (SEQ ID NO 58) | (SEQ ID NO 70) |
| | 2 3′:CAGGTTAGCAGCCAGCTGTTCCCAACCACGCGGCTG | |
| | (SEQ ID NO 59) | |
| F-CB-CD- | 1 5′:TTCGCTGCTAACGTCTATAACACCGTATCTTTTAACCTG | TRHRQPR↓GWE |
| Leg | (SEQ ID NO 60) | QLG↓FGSTF↓FA |
| | 2 3′:GTTATACAGGTTAGCAGCGAAACCCGCGAAGAAGGTAGAAC | GFAAN↓L |
| | C | (SEQ ID NO: 71) |
| | (SEQ ID NO 61) | |
| CL-Leg: | 1 5′:GAATGCGCTAACCTGTATAACACCGTATCTTTTAACCTG | AAN↓L |
| | (SEQ ID NO 62) | (SEQ ID NO 72) |
| | 2 3′:GTTATACAGGTTAGCAGCGCATTCGCCACGGTTAAAGGATT | |
| | T-3′ | |
| | (SEQ ID NO 63) | |
| F-CB-CD | 1 5′:GGTTCTACCTTCTTCGCGGGTTTCTATAACACCGTATCTTTT | TRHRQPR↓GWE |
| | AACCTG | QLG↓FGSTF↓FA |
| | (SEQ ID NO 64) | GF |
| | 2 3′:ACCCGCGAAGAAGGTAGAACCGAAACCCAGCTGTTCCCAAC | (SEQ ID NO 73) |
| | CACGCGGCTG | |
| | (SEQ ID NO 65) | |
| CL-CB | 1 5′:GGTTTCGGTTCTGTTCAGTTCGCGGGTTTCTATAACACCGTA | G↓FGSVQ↓FAGF |
| | TCTTTTAACCTG | (SEQ ID NO 74) |
| | (SEQ ID NO 66) | |
| | 2 3 :GAAACCCGCGAACTGAACAGAACCGAAACCGCATTCGCCAC | |
| | GGTTAAAGGATTT | |
| | (SEQ ID NO 67) | |
| CL-CD | 1 5′GAATGCACCTTCTTCGCGGGTTTCTATAACACCGTATCTTTTA | TF↓FAGF |
| | ACCTG | (SEQ ID NO 75) |
| | (SEQ ID NO 68) | |
| | 2 3′GTTATAGAAACCCGCGAAGAAGGTGCATTCGCCACGGTTAA | |
| | AGGATTT | |
| | (SEQ ID NO 69) | |

F: Furin, GB-CD: Cathepsin B and D, Leg: Legumain. The arrow indicates the cleavage site.

TABLE 5
IC50 of VB6-845-F-de-bouganin and VB6-845-L-de-bouganin
linker constructs in cell lines

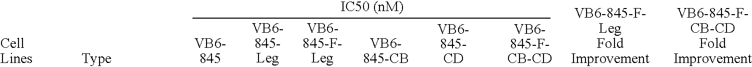

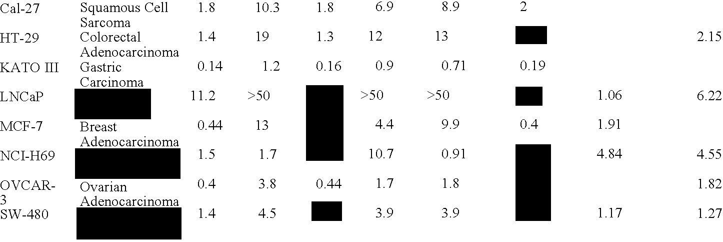

Greyed values indicate IC50 improved in linker with multiple sites. Leg = legumain. F = furin, CB = cathepsin B, CD = cathepsin D.

TABLE 6
Description of Sequences

| SEQ | |
|---|---|
| ID # | Description |
| 1 | Nucleotide sequence of unoptimized VB6-845 |
| 2 | Amino Acid sequence of unoptimized VB6-845 |
| 3 | Nucleotide sequence of unoptimized VB6-845-Ala70 |
| 4 | Amino Acid sequence of unoptimized VB6-845-Ala70 |
| 5 | Primer #1, 5′-SacI-C_{L}-boug, |
| | (5′-TACGCGTGCGAAGTCACCCATCAGGGCCTGAGCTCGGGCGTC-3′) |
| 6 | Primer #2, boug-Ala70-3′, |
| | (5′-GTCTTGATAACCCACAACTGGGACATCTGTCAC-3′) |
| 7 | Primer #3, 5′-boug-Ala70, |
| | (5′-GTGACAGATGTG+E,UN GCA+EE GTTGTGGGTTATGAA-3′) |
| 8 | Primer #4, stop-XhoI-3′, |
| | (5′-GTCGAGCTAGTATTTGGAGCTTTTAAACTTAAGGATAGG-3′) |
| 9 | Nucleotide sequence of unoptimized VB6-845-gelonin |
| 10 | Amino Acid sequence of unoptimized VB6-845-gelonin |
| 11 | Nucleotide sequence of unoptimized VB6-845-pe |
| 12 | Amino Acid sequence of unoptimized VB6-845-pe |
| 13 | 1-5′TAC GCC TGC GAA GTC ACC CAT CAG GGC CTG AGC TCG CCC GTCG |
| 14 | 2-5′:ACC AGG CAC AGG CAT AAA AGA TCC GTC TAC |
| 15 | 3-3′:GTT GTA GAC GGA TCT TTT ATG CCT GTG CCT |
| 16 | Linker F1 TRHRHKR↓SV amino acid sequence |
| 17 | 2-5′:ACC AGG CAC AGG AGG AAA AGA TCC GTC TAC AAC ACC GTG TCA |
| | TTT AAC CTT GGA GAA |
| 18 | 3-3′:GAC GGA TCT TTT CCT CCT GTG CCT GGT ACA CTC TCC CCT GTT |
| | GAA |
| 19 | Linker F1 R TRHRRKR↓SV |
| 20 | 2-5′:CAG GGT GTA GGA TTT GGC ACC CAG TTT TTC TAC AAC ACC GTG |
| | TCA TTT AAC CTT GGA |
| 21 | 3-3′:CTG GGT GCC AAA TCC TAC ACC CTG ACA CTC TCC CCT GTT GAA |
| | GCT CTT |
| 22 | Linker CB-CD QGVGF↓GTQF↓ |
| 23 | 2-5′:GGA TTT CTA GGC ACC CAG TTT TTC TAC AAC ACC |
| 24 | 3-3′:GAA AAA CTG GGT GCC TAG AAA TCC TAC ACC |
| 25 | Linker CB1-CD QGVGF↓LGTQF↓F |
| 26 | 2-5′:CAG GGT GTA GGA TTT GGC ACC CAG TTT TTC TAC AAC ACC GTG |
| | TCA TTT AAC CTT GGA |
| 27 | 3-3′:CTG GGT GCC AAA TCC TAC ACC CTG GAG CTG CTC CCA GCC TCT |
| | GGG CTG CCT GTG CCT GGT |
| 28 | Linker F-CBCGD TRHRQPR↓GWEQL QGVGF↓GTQF↓F |
| 29 | 2-5′:CAG GGT GTA GGA TTT GGC ACC CAG TTT TTC TAC AAC ACC GTG |
| | TCA TTT AAC CTT GGA |
| 30 | 3-3′:CTG GGT GCC AAA TCC CAT ACC CTG GAC GGA TCT TTT ATG CCT |
| | GTG CCT GGT ACA CTC TCC CCT GTT GAA |
| 31 | Linker F1-CB-CD TRHRHKR↓SV QGVGF↓GTQF↓F |
| 32 | 2-5′:CAG GGT GTA GGA TTT GGC ACC CAG TTT TTC TAC AAC ACC GTG |
| | TCA TTT AAC CTT GGA |
| 33 | 3-3′:CTG GGT GCC AAA TCC TAC ACC CTG GAC GGA TCT TTT CCT CCT |
| | GTG CCT GGT AGA CTC TCC CCT GTT GAA |
| 34 | Linker F1R-CB-GD TRHRRKR↓SV QGVGF↓GTQF↓F |
| 35 | 4-5′CTC GAG CTA CTA TTT GGA GCT TTT AAA CTT AAG GAT ACC |
| 36 | Original Furin linker unoptimized nucleotide sequence |
| 37 | Original Furin linker unoptimized amino acid sequence |
| 38 | F1 Furin linker unoptimized nucleotide sequence |
| 39 | F1 Furin linker unoptimized amino acid sequence |
| 40 | F1R Furin linker unoptimized nucleotide sequence |
| 41 | F1R Furin linker unoptimized amino acid sequence |
| 42 | CB-CD linker unoptimized nucleotide sequence |
| 43 | CB-CD linker unoptimized amino acid sequence |
| 44 | CB1-CD linker unoptimized nucleotide sequence |
| 4S | CB1-CD linker unoptimized amino acid sequence |
| 46 | F-CB-CD linker unoptimized nucleotide sequence |
| 47 | F-CB-CD linker unoptimized amino acid sequence |
| 48 | F1-GC-CD linker unoptimized nucleotide sequence |
| 49 | F1-CB-GD linker unoptimized amino acid sequence |
| 50 | F1R-CB-CD linker unoptimized nucleotide sequence |
| 51 | F1R-CB-CD linker unoptimized amino acid sequence |
| 52 | Nucleotide sequence of optimized VB6-845 |
| 53 | Amino Acid sequence of optimized VB6-845 |
| 54 | Gathepsin Site |
| 55 | Legumain Site |
| 56 | 5′CL-Kappa-CODA-Styl 5′TACGCTTGCGAGGTGACCCACCAAGGTCTG |
| 57 | 3′Boug-CODA-XhoI 5′GCGCTCGAGTCACTATTTGCTAGATTTAAATTTCAG |
| 58 | F-Leg 5′:TGGGAAGAGCTGGCTGCTAACCTGTATAACACCGTATCTTTTAACCTG |
| 59 | F-Leg 3′:CAGGTTAGCAGCCAGCTGTTCCCAACCACGCGGCTG |
| 60 | F-CB-CD-Leg 5′:TTCGCTGCTAACGTCTATAACACCGTATCTTTTAACCTG |
| 61 | F-CB-CD-Leg 3′:GTTATACAGGTTAGCAGCGAAACCCGCGAAGAAGGTAGAACC |
| 62 | CL-Leg: 5′:GAATGCGGTAACCTGTATAACACCGTATCTTTTAACCTG |
| 63 | CL-Leg: 3′:GTTATACAGGTTAGCAGCGCATTCGCCACGGTTAAAGGATTT-3′ |
| 64 | F-CB-CD 5′:GGTTCTACCTTCTTCGCGGGTTTCTATAACACCGTATCTTTTAACCTG |
| 65 | F-CB-CD 3′:ACCCGCGAAGAAGGTAGAACCGAAACCCAGCTGTTCCCAACCACGCGGCTG |
| 66 | CL-CB 5′:GGTTTCGGTTCTGTTCAGTTCGCGGGTTTCTATAACACCGTATCTTTTAACCTG |
| 67 | CL-CB 3′:GAAACCCGCGAACTGAACAGAACCGAAACCGCATTCGCCACGGTTAAAGGATTT |
| 68 | CL-CD 5′GAATGCACCTTCTTCGCGGGTTTCTATAACACCGTATCTTTTAACCTG |
| 69 | CL-CD 3′GTTATAGAAACCCGCGAAGAAGGTGCATTCGCCACGGTTAAAGGATTT |
| 70 | TRHRQPR↓G WEQLAAN↓L |
| 71 | TRHRQPR↓GWEQLG↓FGSTF↓FAGFAAN↓L |
| 72 | AAN↓L |
| 73 | TRHRQPR↓GWEQLG↓FGSTF↓FAGF |
| 74 | G↓FGSVQ↓FAGF |
| 75 | TF↓FAGF |
| 76 | Furin linker optimized nucleotide sequence |
| 77 | Furin linker optimized amino acid sequence |
| 78 | F-Leg linker nucleotide sequence |
| 79 | F-Leg linker amino acid sequence |
| 80 | Leg linker nucleotide sequence |
| 81 | Leg linker amino acid sequence |
| 82 | CB linker nucleotide sequence |
| 83 | CB linker amino acid sequence |
| 84 | CD linker nucleotide sequence |
| 85 | CD linker amino acid sequence |
| 86 | F-CB-CD linker nucleotide sequence |
| 87 | F-CB-CD linker amino acid sequence |
| 88 | F-CB-CD-Leg linker nucleotide sequence |
| 89 | F-CB-CD-Leg linker amino acid sequence |
| 90 | De-bouganin |

### FULL CITATIONS FOR REFERENCES REFERRED TO IN THE SPECIFICATION

1. Barrand, M. A., Bagrij, T., and Neo, S. Y. (1997). Multidrug resistance-associated protein: a protein distinct from P-glycoprotein involved in cytotoxic drug expulsion. Gen. Pharmacol. 28, 639-645.
2. Beste, G., Schmidt, F. S., Stibora, T., and Skerra, A. (1999). Small antibody-like proteins with prescribed ligand specificities derived from the lipocalin fold. Proc Natl. Acad. Sci. U.S. A 96, 1898-1903.
3. Blaustein, R. O., Koehler, T. M., Collier, R. J., and Finkelstein, A. (1989). Anthrax toxin: channel-forming activity of protective antigen in planar phospholipid bilayers. Proc Natl. Acad. Sci. U.S. A 86, 2209-2213.
4. Bolognesi, A., Polito, L., Olivieri, F., Valbonesi, P., Barbieri, L., Battelli, M. G., Carusi, M. V., Benvenuto, E., del Vecchio, B. F., Di, M. A., Parente, A., Di, L. M., and Stirpe, F. (1997). New ribosome-inactivating proteins with polynucleotide:adenosine glycosidase and antiviral activities from Basella rubra L. and bougainvillea spectabilis Willd. Planta 203, 422-429.
5. Boquet, P., Silverman, M. S., Pappenheimer, A. M., Jr., and Vernon, W. B. (1976). Binding of triton X-100 to diphtheria toxin, crossreacting material 45, and their fragments. Proc Natl. Acad. Sci. U.S. A 73, 4449-4453.
6. Boss, M. A., Kenten, J. H., Emtage, J. S., and Wood, C. R. Multichain polypeptides or proteins and processes for their production. Celltech, Limited. 000672265[4816397], 1-26. 3-28-1989. U.S. Pat. Nos. 4,403,036, 4,642,334. 11-14-1984.
Ref Type: Patent
7. Bruhn, H., Funk, M., and Henkel, T. NOVEL SYNTHETIC PROTEIN STRUCTURAL TEMPLATES FOR THE GENERATION, SCREENING AND EVOLUTION OF FUNCTIONAL MOLECULAR SURFACES. STEIPE, Boris. EP0002840[9745538], 1-137. 12-4-1997. WO. 5-30-1997.
Ref Type: Patent
8. Cabilly, S., Heyneker, H. L., Holmes, W. E., Riggs, A. D., and Wetzel, R. B. Recombinant immunoglobin preparations. Genentech, Inc. 000483457[4816567], 1-21. 3-28-1989. U.S. Pat. Nos. 4,444,878, 4,512,922, 4,518,584, 4,704,362. 4-8-1983.
Ref Type: Patent
9. Chahal, F. C., MacDonald G. C, and Cizeau, J. Novel Cancer Associated Antigen. Viventia Biotech. WO07071051A1. 6-28-2007.
Ref Type: Patent
10. Deeks, E. D., Cook, J. P., Day, P. J., Smith, D. C., Roberts, L. M., and Lord, J. M. (2002). The low lysine content of ricin A chain reduces the risk of proteolytic degradation after translocation from the endoplasmic reticulum to the cytosol. Biochemistry 41, 3405-3413.
11. den Hartog, M. T., Lubelli, C., Boon, L., Heerkens, S., Ortiz Buijsse, A. P., de, B. M., and Stirpe, F. (2002). Cloning and expression of cDNA coding for bouganin. Eur. J. Biochem. 269, 1772-1779.
12. DeNardo, G. L., Denardo, S. J., Peterson, J. J., Miers, L. A., Lam, K. S., Hartmann-Siantar, C., and Lamborn, K. R. (2003). Preclinical evaluation of cathepsin-degradable peptide linkers for radioimmunoconjugates. Clin Cancer Res 9, 3865S-3872S.
13. Efuet, E. T. and Keyomarsi, K., (2006). Farnesyl and Geranylgeranyl Transferase Inhibitors Induce G₁ Arrest by Targeting the Proteasome. Cancer Res 66, 1040-1051.
14. Eklund, N., Axelsson, L., Uhlen, M., and Nygren, P.-A. (2002). Anti-idiotypic protein domains selected from protein A-based affibody libraries. Proteins: Structure, Function, and Genetics 48, 454-462.
15. Forrer, P., Stumpp, M. T., Binz, H. K., and Pluckthun, A. (2003). A novel strategy to design binding molecules harnessing the modular nature of repeat proteins. FEBS Lett. 539, 2-6.
16. Fuchs, H., Sutherland, M., Bachran, C., Melzig, M., Heisler, I., and Hebestreit, P. A COMPOSITION COMPRISING A PHARMACOLOGICALLY ACTIVE AGENT COUPLED TO A TARGET CELL SPECIFIC COMPONENT, AND A SAPONIN. CHARITE-UNIV and FREIE UNIV. EP0007557[6005581], 1-40. 1-19-2006. WO. 7-12-2005.
Ref Type: Patent
17. Gennaro, A. R. (2000). Remington's Pharmaceutical Sciences. (Easton, Pa.: Mack Publishing Company).
18. Glover, N., MacDonald G. C, Cizeau, J, Entwistle, J., and Chahal, F. C. Cancer Specific Antibody and Cell Surface Proteins. Viventia Biotech. WO06066408A1. 6-29-2006.
Ref Type: Patent
19. Glover, N., MacDonald G. C, Entwistle, J., Cizeau, J, Bosc, D, and Chahal, F. C. Tumor Specific Antibody. Viventia Biotech. WO0512134A1. 12-22-2005. 6-10-2005.
Ref Type: Patent
20. Gotz, M., Hess, S., Beste, G., Skerra, A., and Michel-Beyerle, M. E. (2002). Ultrafast electron transfer in the complex between fluorescein and a cognate engineered lipocalin protein, a so-called anticalin. Biochemistry 41, 4156-4164.
21. Gunneriusson, E., Nord, K., Uhlen, M., and Nygren, P. (1999). Affinity maturation of a Taq DNA polymerase specific affibody by helix shuffling. Protein Eng 12, 873-878.
22. Hansson, M., Ringdahl, J., Robert, A., Power, U., Goetsch, L., Nguyen, T. N., Uhlen, M., Stahl, S., and Nygren, P. A. (1999). An in vitro selected binding protein (affibody) shows conformation-dependent recognition of the respiratory syncytial virus (RSV) G protein. Immunotechnology. 4, 237-252.
23. Heisler, I., Keller, J., Tauber, R., Sutherland, M., and Fuchs, H. (2003). A cleavable adapter to reduce nonspecific cytotoxicity of recombinant immunotoxins. Int. J. Cancer 103, 277-282.
24. Henning, P., Magnusson, M. K., Gunneriusson, E., Hong, S. S., Boulanger, P., Nygren, P. A., and Lindholm, L. (2002). Genetic modification of adenovirus 5 tropism by a novel class of ligands based on a three-helix bundle scaffold derived from staphylococcal protein A. Hum. Gene Ther. 13, 1427-1439.
25. Hogbom, M., Eklund, M., Nygren, P. A., and Nordlund, P. (2003). Structural basis for recognition by an in vitro evolved affibody. Proc Natl. Acad. Sci. U.S. A 100, 3191-3196.
26. John Wiley & Sons (2006). Current Protocols in Molecular Biology. (New York, N.Y.: John Wiley & Sons).
27. Keller, J., Heisler, I., Tauber, R., and Fuchs, H. (2001). Development of a novel molecular adapter for the optimization of immunotoxins. J Control Release 74, 259-261.
28. Kohl, A., Binz, H. K., Forrer, P., Stumpp, M. T., Pluckthun, A., and Grutter, M. G. (2003). Designed to be stable: crystal structure of a consensus ankyrin repeat protein. Proc Natl. Acad. Sci. U.S. A 100, 1700-1705.
29. Kreitman, R. J. and Pastan, I. (1995). Importance of the glutamate residue of KDEL in increasing the cytotoxicity of Pseudomonas exotoxin derivatives and for increased binding to the KDEL receptor. Biochem. J. 307 (Pt 1), 29-37.
30. Lipovsek, D. PROTEIN SCAFFOLDS FOR ANTIBODY MIMICS AND OTHER BINDING PROTEINS. PHYLOS, INC. US0029317[34784], 1-56. 6-15-2000. WO. 12-9-1999.
Ref Type: Patent
31. Lipovsek, D., Wagner, R., and Kuimelis, R. PROTEIN SCAFFOLDS FOR ANTIBODY MIMICS AND OTHER BINDING PROTEINS. PHYLOS, INC. US0006414[164942], 1-67. 9-7-2001. WO. 2-28-2001.
Ref Type: Patent
32. Lipovsek, D., Wagner, R., and Kuimelis, R. PROTEIN SCAFFOLDS FOR ANTIBODY MIMICS AND OTHER BINDING PROTEINS. PHYLOS, INC. US0032233[232925], 1-94. 4-25-2002. WO. 10-16-2001.
Ref Type: Patent
33. Liu, C., Sun, C., Huang, H., Janda, K., and Edgington, T. (2003). Overexpression of legumain in tumors is significant for invasion/metastasis and a candidate enzymatic target for prodrug therapy. Cancer Res 63, 2957-2964.
34. Monzingo, A. F. and Robertus, J. D. (1992). X-ray analysis of substrate analogs in the ricin A-chain active site. J. Mol. Biol. 227, 1136-1145.
35. Morris, K. N. and Wool, I. G. (1992). Determination by systematic deletion of the amino acids essential for catalysis by ricin A chain. Proc Natl. Acad. Sci. U.S. A 89, 4869-4873.
36. Morrison, S. L., Herzenberg, L. A., and Oi, V. T. Chimeric receptors by DNA splicing and expression. THE BOARD OF TRUSTEES OF THE LELAND STANFORD JUNIOR UNIVERSITY. 000305604[173494], 1-26. 3-5-1986. EP. 8-7-1985.
Ref Type: Patent
37. Morrison, S. L., Johnson, M. J., Herzenberg, L. A., and Oi, V. T. (1984). Chimeric human antibody molecules: mouse antigen-binding domains with human constant region domains. Proc Natl. Acad. Sci. U.S. A 81, 6851-6855.
38. Neuberger, M. S, and Rabbitts, T. H. PRODUCTION OF CHIMERIC ANTIBODIES. * CELLTECH LIMITED. 008608827[2177096], 1. 5-17-1989. GB. 4-11-1986.
Ref Type: Patent
39. Nilsson, B., Nygren, P., and Uhlen, M. Bacterial receptor structures. Upjohn, Aktiebolag. 000669360[5831012], 1-29. 11-3-1998. U.S. Pat. Nos. 4,879,213, 4,954,618, 5,084,559. 8-15-1996.
Ref Type: Patent
40. Nilsson, B., Nygren, P., and Uhlen, M. Bacterial receptor structures. Biovitrum, A. B. 000082468[6534628], 1-30. 3-18-2003. U.S. Pat. Nos. 4,879,213, 4,954,618, 5,084,559, 5,229,492, 5,312,901, 5,783,415, 5,831,012, 6,025,166, 6,027,927. 5-21-1998.
Ref Type: Patent
41. Nord, K., Gunneriusson, E., Ringdahl, J., Stahl, S., Uhlen, M., and Nygren, P. A. (1997). Binding proteins selected from combinatorial libraries of an alpha-helical bacterial receptor domain. Nat. Biotechnol. 15, 772-777.
42. Nord, K., Gunneriusson, E., Uhlen, M., and Nygren, P. A. (2000). Ligands selected from combinatorial libraries of protein A for use in affinity capture of apolipoprotein A-1M and taq DNA polymerase. J Biotechnol. 80, 45-54.
43. Nord, K., Nilsson, J., Nilsson, B., Uhlen, M., and Nygren, P. A. (1995). A combinatorial library of an alpha-helical bacterial receptor domain. Protein Eng 8, 601-608.
44. Nord, K., Nord, O., Uhlen, M., Kelley, B., Ljungqvist, C., and Nygren, P. A. (2001). Recombinant human factor VIII-specific affinity ligands selected from phage-displayed combinatorial libraries of protein A. Eur. J. Biochem. 268, 4269-4277.
45. Nygren, P. A. and Uhlen, M. (1997). Scaffolds for engineering novel binding sites in proteins. Curr. Opin. Struct. Biol. 7, 463-469.
46. Pluckthun, A., Honegger, A., and Willuda, J. Method for the Stabilization of Chimeric Immunoglobulins or Immunoglobulin Fragments and Stabilized Anti EGP-2 SCFV Fragment. PCT/EP00/03176, 1-123. 2006. WO. 4-9-1999.
Ref Type: Patent
46. Rajamohan, F., Pugmire, M. J., Kurinov, I. V., and Uckun, F. M. (2000). Modeling and alanine scanning mutagenesis studies of recombinant pokeweed antiviral protein. J. Biol. Chem. 275, 3382-3390.
47. Rao, S., Porter, D. C., Chen, X., Herliczek, T., Lowe, M. and Keyomarsi, K., (1999). Lovastatin-mediated G₁ arrest is through inhibition of the proteasome, independent of hydroxymethyl glutaryl-CoA reductase. Proc. Natl. Acad. Sci. USA, Vol. 96, 7797-7802.
48. Ready, M. P., Kim, Y., and Robertus, J. D. (1991). Site-directed mutagenesis of ricin A-chain and implications for the mechanism of action. Proteins 10, 270-278.
49. Rodighiero, C., Tsai, B., Rapoport, T. A., and Lencer, W. I. (2002). Role of ubiquitination in retro-translocation of cholera toxin and escape of cytosolic degradation. EMBO Rep. 3, 1222-1227.
50. Ronnmark, J., Gronlund, H., Uhlen, M., and Nygren, P. A. (2002a). Human immunoglobulin A (IgA)-specific ligands from combinatorial engineering of protein A. Eur. J. Biochem. 269, 2647-2655.
51. Ronnmark, J., Hansson, M., Nguyen, T., Uhlen, M., Robert, A., Stahl, S., and Nygren, P. A. (2002b). Construction and characterization of affibody-Fc chimeras produced in Escherichia coli. J Immunol Methods 261, 199-211.
52. Sandvig, K. and van Deurs, B. (2002). Membrane traffic exploited by protein toxins. Annu. Rev. Cell Dev. Biol. 18, 1-24.
53. Schlehuber, S., Beste, G., and Skerra, A. (2000). A novel type of receptor protein, based on the lipocalin scaffold, with specificity for digoxigenin. J. Mol. Biol. 297, 1105-1120.
54. Selbo, P. K., Sivam, G., Fodstad, O., Sandvig, K., and Berg, K. (2000). Photochemical internalisation increases the cytotoxic effect of the immunotoxin MOC31-gelonin. Int. J. Cancer 87, 853-859.
55. Skerra, A. (2000a). Engineered protein scaffolds for molecular recognition. J. Mol. Recognit. 13, 167-187.
56. Skerra, A. (2000b). Lipocalins as a scaffold. Biochim. Biophys. Acta 1482, 337-350.
57. Skerra, A. (2001). ‘Anticalins’: a new class of engineered ligand-binding proteins with antibody-like properties. J Biotechnol. 74, 257-275.
58. Smith, D. C., Spooner, R. A., Watson, P. D., Murray, J. L., Hodge, T. W., Amessou, M., Johannes, L., Lord, J. M., and Roberts, L. M. (2006). Internalized Pseudomonas exotoxin A can exploit multiple pathways to reach the endoplasmic reticulum. Traffic. 7, 379-393.
59. Spooner, R. A., Smith, D. C., Easton, A. J., Roberts, L. M., and Lord, J. M. (2006). Retrograde transport pathways utilised by viruses and protein toxins. Virol. J 3, 26.
60. Takeda, S., Naito, T., Hama, K., Noma, T., and Honjo, T. (1985). Construction of chimaeric processed immunoglobulin genes containing mouse variable and human constant region sequences. Nature 314, 452-454.
61. Taniguchi, M., Kurosawa, Y., and Sugita, K-Z. A chimera monoclonal antibody and a production process thereof. Research Development Corporation of Japan. 000102665[171496], 1-22. 2-19-1986. EP. 3-8-1985.
Ref Type: Patent
62. Tavare, J. M., Fletcher, L. M., and Welsh, G. I. (2001). Using green fluorescent protein to study intracellular signalling. J Endocrinol. 170, 297-306.
63. Teter, K. and Holmes, R. K. (2002). Inhibition of endoplasmic reticulum-associated degradation in CHO cells resistant to cholera toxin, Pseudomonas aeruginosa exotoxin A, and ricin. Infect. Immun. 70, 6172-6179.
64. Tsai, B., Rodighiero, C., Lencer, W. I., and Rapoport, T. A. (2001). Protein disulfide isomerase acts as a redox-dependent chaperone to unfold cholera toxin. Cell 104, 937-948.
65. Tsuruo, T., Naito, M., Tomida, A., Fujita, N., Mashima, T., Sakamoto, H., and Haga, N. (2003). Molecular targeting therapy of cancer: drug resistance, apoptosis and survival signal. Cancer Sci. 94, 15-21.
66. Vago, R., Marsden, C. J., Lord, J. M., Ippoliti, R., Flavell, D. J., Flavell, S. U., Ceriotti, A., and Fabbrini, M. S. (2005). Saporin and ricin A chain follow different intracellular routes to enter the cytosol of intoxicated cells. FEBS J 272, 4983-4995.
67. Wahlberg, E., Lendel, C., Helgstrand, M., Allard, P., ncbas-Renqvist, V., Hedqvist, A., Berglund, H., Nygren, P. A., and Hard, T. (2003). An affibody in complex with a target protein: structure and coupled folding. Proc Natl. Acad. Sci. U.S. A 100, 3185-3190.
68. Winter, G. P. Recombinant antibodies and methods for their production Winter, Gregory Paul. 000302620[239400], 1-41. 9-30-1987. EP. 3-26-1987.
Ref Type: Patent

1. A conjugate comprising:
(a) a ligand that binds to a surface molecule on a target cell;
(b) an effector molecule; and
(c) a linker that couples the ligand and the effector molecule, the linker comprising at least one protease cleavage site corresponding to a protease found in an intracellular trafficking pathway of the effector molecule;
wherein cleavage of the linker by the protease uncouples the effector from the ligand.

2. A conjugate according to claim 1 wherein the ligand is an antibody or antibody fragment.

3. A conjugate according to claim 1 wherein the effector molecule is a cancer therapeutic.

4. A conjugate according to claim 3 wherein the cancer therapeutic is a toxin.

5. A conjugate according to claim 4 wherein the toxin is selected from the group comprising agents that disrupt DNA, agents that disrupt tubulin, agents that are antimitotic, topoisomerase I inhibitors, topoisomerase II inhibitors, RNA or DNA antimetabolites and ribosome inactivating proteins.

6. A conjugate of claim 4 wherein the toxin is a ribosome inactivating protein.

7. A conjugate of claim 4, wherein the toxin is selected from the group consisting of gelonin, bouganin, saporin, ricin, ricin A chain, bryodin, diphtheria, restrictocin and Pseudomonas exotoxin A or variants thereof.

8. A conjugate according to claim 6 wherein the toxin is bouganin, modified bouganin or a variant thereof.

9. A conjugate according to claim 8 wherein the modified bouganin is a de-bouganin.

10. A conjugate according to claim 6 wherein the toxin is a truncated form of Pseudomonas exotoxin A that consists of amino acids 252-608 or a variant thereof.

11. A conjugate according to claim 1 wherein the ligand binds to Ep-CAM.

12. A conjugate according to claim 1 wherein the linker comprises a lysosome or endosome protease specific site.

13. A conjugate according to claim 1 wherein the linker comprises a cathepsin specific site.

14. A conjugate according to claim 1 wherein the linker comprises a leugamain specific site.

15. A conjugate of claim 13 wherein the linker further comprises a furin specific site.

16. A conjugate according to claim 1 wherein the linker is cleaved by one or more proteases selected from the group consisting of furin, cathepsins, matrix metalloproteinases and legumain.

17. A conjugate according to claim 1 wherein the linker comprises at least two protease cleavage sites.

18. A conjugate according to claim 17 wherein the at least two protease cleavage sites comprise a furin cleavage site and a cathepsin cleavage site.

19. A conjugate according to claim 1 encoded by a nucleic acid comprising:
the nucleic acid sequence of SEQ ID NO:1 with a substitution at positions 1607-1642 with a nucleic acid sequence consisting of SEQ ID NO:38, 40, 42, 44, 46, 48 or 50; or
the nucleic acid sequence of SEQ ID NO:52 with a substitution at positions 1557-1592 with a nucleic acid sequence consisting of SEQ ID NO:76, 78, 80, 82, 84, 86 or 88.

20. A conjugate according to claim 1 comprising:
the amino acid sequence of SEQ ID NO:2 with a substitution at positions 489-500 with a linker consisting of the amino acid sequence of SEQ ID NOS: 39, 41, 43, 45, 47, 49, or 51; or
the amino acid sequence of SEQ ID NO:53 with a substitution at positions 489-500 with a linker consisting of the amino acid sequence of SEQ ID NOS: 79, 81, 83, 85, 87, or 89.

21. An isolated nucleic acid sequence encoding the conjugate according to claim 1.

22. An isolated nucleic acid sequence comprising:
the nucleic acid sequence of SEQ ID NO:1 with a substitution at positions 1607-1642 with a nucleic acid sequence consisting of SEQ ID NO:38, 40, 42, 44, 46, 48 or 50; or
the nucleic acid sequence of SEQ ID NO:52 with a substitution at positions 1557-1592 with a nucleic acid sequence consisting of SEQ ID NO:76, 78, 80, 82, 84, 86 or 88.

23. A recombinant expression vector comprising the nucleic acid sequence of claim 21.

24. A host cell comprising the recombinant expression vector of claim 23.

25. A pharmaceutical composition comprising a conjugate according to claim 1 with a pharmaceutically acceptable excipient, diluent, carrier, buffer or stabilizer.

26. A method of treating or preventing cancer comprising administering an effective amount of the conjugate according to claim 1 to a subject in need thereof.

27. A method of treating or preventing cancer comprising administering the pharmaceutical composition of claim 25 to a subject in need thereof.

28. The method of claim 26, further comprising the administration of at least one additional anticancer therapy.

29. The method of claim 28, wherein the additional anticancer therapy comprises epoxomicin, MG-132, lactacystin, trichostatin A, curcumin, proteasome inhibitor I, chymostatin, lovastatin, simvastatin, FTI-277, GGTI-298, ascorbic acid, acetylsalicyclic acid or butyrolactone, and the effector molecule comprises Pseudomonas exotoxin A or a truncated form of Pseudomonas exotoxin A that consists of amino acids 252-608.

30-33. (canceled)

34. A kit for treating or preventing cancer comprising the conjugate of claim 1 and directions for the use thereof to treat or prevent cancer.

35. A diagnostic agent comprising the conjugate of claim 1, wherein the effector molecule is a label, which can generate a detectable signal, directly or indirectly.

36. A method of detecting or monitoring cancer in a subject comprising the steps of
(1) contacting a test sample from said subject with the diagnostic agent of claim 35 that binds specifically to a cancer cell to produce conjugate-antigen complex;
(2) measuring the amount of conjugate-antigen complex in the test sample; and
(3) comparing the amount of conjugate-antigen complex in the test sample to a control.

37. (canceled)


SEQUENCE LISTING (90 sequences)

SEQ ID NO: 1 - DNA, 2401 bp, Artificial Sequence
  unoptimized VB6-845
gaattcctgcaggtctatggaacgataaatgcccatgaaaattctatttcaaggagacagtcataatgaaatacctattgcctacggcagccgctggattgttattactcgctgcccaaccagcgatggcgcaccatcatcaccatcacgaagtacagctggttcagtccggcccgggtcttgttcaaccgggtggttccgttcgtatctcttgcgctgcttctggttacacgttcaccaactacggcatgaactgggtcaaacaggctccgggtaaaggcctggaatggatgggctggatcaacacctacaccggtgaatccacctacgctgactccttcaaaggtcgcttcactttctccctcgacacaagtgctagtgctgcatacctccaaatcaactcgctgcgtgcagaggatacagcagtctattactgcgcccgtttcgctatcaaaggtgactactggggtcaaggcacgctgctgaccgtttcctcggctagcaccaaaggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttgatctagagtcgacctgcaggtctatggaacgataaatgcccatgaaaattctatttcaaggagacagtcataatgaaatacctattgcctacggcagccgctggattgttattactcgctgcccaaccagcgatggcggatatccagatgacccagtccccgtcctccctgagtgcttctgttggtgaccgtgttaccatcacctgccgttccaccaaatccctcctgcactccaacggtatcacctacctttattggtatcaacagaaaccgggtaaagctccgaaacttctgatctaccagatgtccaacctggcttccggtgttccgtctcgtttctccagttctggttctggtaccgacttcaccctgaccatctcttctctgcagccggaagacttcgctacctactactgcgctcagaacctggaaatcccgcgtaccttcggtcagggtaccaaagttgaacttaagcgcactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtaccaggcacaggcagcccagaggctgggagcagctctacaacaccgtgtcatttaaccttggagaagcttatgagtaccccacttttatacaagatttgcgcaatgaattggctaagggcacaccagtatgtcaacttccagtgacactacaaaccatagccgatgacaagcgatttgttctagttgatatcactacgacctcgaagaaaacagttaaggttgctatagatgtgacagatgtgtatgttgtgggttatcaagacaaatgggatggcaaagatcgagctgttttccttgacaaggttcctactgttgcaactagtaaacttttcccaggggtgactaatcgtgtaacgttaacatttgatggcagctatcagaaacttgtgaatgctgccaaagctgatagaaaggctctcgaactgggggttaacaaattggaattttccattgaagcaatccatggtaaaacgataaatggtcaagaggcagccaagttctttcttattgtcatccaaatggtttcagaggcagctcggttcaaatatattgagactgaggtggttgatagaggattatatggatcattcaaacctaattttaaagtattgaacttggagaacaattggggcgacatctctgatgccattcacaaatcatccccacaatgtaccactattaatccggcacttcagttgataagcccctcaaatgacccatgggttgtaaataaagtgagtcaaattagtcccgatatgggtatccttaagtttaaaagctccaaatagtgactcgag

SEQ ID NO: 2 - protein, 750 aa, Artificial Sequence
  unoptimized VB6-845
MKYLLPTAAAGLLLLAAQPAMAHHHHHHEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCMKYLLPTAAAGLLLLAAQPAMADIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTRHRQPRGWEQLYNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKADRKALELGVNKLEFSIEAIHGKTINGQEAAKFFLIVIQMVSEAARFKYIETEVVDRGLYGSFKPNFKVLNLENNWGDISDAIHKSSPQCTTINPALQLISPSNDPWVVNKVSQISPDMGILKFKSSK

SEQ ID NO: 3 - DNA, 2404 bp, Artificial Sequence
  unoptimized VB6-845-Ala70
gaattcctgcaggtctatggaacgataaatgcccatgaaaattctatttcaaggagacagtcataatgaaatacctattgcctacggcagccgctggattgttattactcgctgcccaaccagcgatggcgcaccatcatcaccatcacgaagtacagctggttcagtccggcccgggtcttgttcaaccgggtggttccgttcgtatctcttgcgctgcttctggttacacgttcaccaactacggcatgaactgggtcaaacaggctccgggtaaaggcctggaatggatgggctggatcaacacctacaccggtgaatccacctacgctgactccttcaaaggtcgcttcactttctccctcgacacaagtgctagtgctgcatacctccaaatcaactcgctgcgtgcagaggatacagcagtctattactgcgcccgtttcgctatcaaaggtgactactggggtcaaggcacgctgctgaccgtttcctcggctagcaccaaaggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgttagtgatctagagtcgacctgcaggtctatggaacgataaatgcccatgaaaattctatttcaaggagacagtcataatgaaatacctattgcctacggcagccgctggattgttattactcgctgcccaaccagcgatggcggatatccagatgacccagtccccgtcctccctgagtgcttctgttggtgaccgtgttaccatcacctgccgttccaccaaatccctcctgcactccaacggtatcacctacctttattggtatcaacagaaaccgggtaaagctccgaaacttctgatctaccagatgtccaacctggcttccggtgttccgtctcgtttctccagttctggttctggtaccgacttcaccctgaccatctcttctctgcagccggaagacttcgctacctactactgcgctcagaacctggaaatcccgcgtaccttcggtcagggtaccaaagttgaacttaagcgcactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgtaccaggcacaggcagcccagaggctgggagcagctctacaacaccgtgtcatttaaccttggagaagcttatgagtaccccacttttatacaagatttgcgcaatgaattggctaagggcacaccagtatgtcaacttccagtgacactacaaaccatagccgatgacaagcgatttgttctagttgatatcactacgacctcgaagaaaacagttaaggttgctatagatgtgacagatgtggcagttgtgggttatcaagacaaatgggatggcaaagatcgagctgttttccttgacaaggttcctactgttgcaactagtaaacttttcccaggggtgactaatcgtgtaacgttaacatttgatggcagctatcagaaacttgtgaatgctgccaaagctgatagaaaggctctcgaactgggggttaacaaattggaattttccattgaagcaatccatggtaaaacgataaatggtcaagaggcagccaagttctttcttattgtcatccaaatggtttcagaggcagctcggttcaaatatattgagactgaggtggttgatagaggattatatggatcattcaaacctaattttaaagtattgaacttggagaacaattggggcgacatctctgatgccattcacaaatcatccccacaatgtaccactattaatccggcacttcagttgataagcccctcaaatgacccatgggttgtaaataaagtgagtcaaattagtcccgatatgggtatccttaagtttaaaagctccaaatagtgactcgag

SEQ ID NO: 4 - protein, 750 aa, Artificial Sequence
  unoptimized VB6-845-Ala70
MKYLLPTAAAGLLLLAAQPAMAHHHHHHEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCMKYLLPTAAAGLLLLAAQPAMADIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECTRHRQPRGWEQLYNTVSFNLGEAYEYPTFIQDLRNELALQKGTPVCQLPVTTIADDKRFVLVDITTTSKKTVKVAIDVTDVAVVGYQDKWDGKDRAVFLDKVPTVATSKLFPGVTNRVTLTFDGSYQKLVNAAKADRKALELGVNKLEFSIEAIHGKTINGQEAAKFFLIVIQMVSEAARFKYIETEVVDRGLYGSFKPNFKVLNLENNWGDISDAIHKSSPQCTTINPALQLISPSNDPWVVNKVSQISPDMGILKFKSSK

SEQ ID NO: 5 - DNA, 42 bp, Artificial Sequence
  Primer
tacgcctgcgaagtcacccatcagggcctgagctcgcccgtc

SEQ ID NO: 6 - DNA, 33 bp, Artificial Sequence
  Primer
gtcttgataacccacaactgccacatctgtcac

SEQ ID NO: 7 - DNA, 30 bp, Artificial Sequence
  Primer
gtgacagatgtggcagttgtgggttatcaa

SEQ ID NO: 8 - DNA, 39 bp, Artificial Sequence
  Primer
ctcgagctactatttggagcttttaaacttaaggatacc

SEQ ID NO: 9 - DNA, 2401 bp, Artificial Sequence
  unoptimized VB6-845-gelonin
gaattcctgcaggtctatggaacgataaatgcccatgaaaattctatttcaaggagacagtcataatgaaatacctattgcctacggcagccgctggattgttattactcgctgcccaaccagcgatggcggaagtacagctggttcagtccggcccgggtcttgttcaaccgggtggttccgttcgtatctcttgcgctgcttctggttacacgttcaccaactacggcatgaactgggtcaaacaggctccgggtaaaggcctggaatggatgggctggatcaacacctacaccggtgaatccacctacgctgactccttcaaaggtcgcttcactttctccctcgacacaagtgctagtgctgcatacctccaaatcaactcgctgcgtgcagaggatacagcagtctattactgcgcccgtttcgctatcaaaggtgactactggggtcaaggcacgctgctgaccgtttcctcggcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtaccaggcacaggcagcccagaggctgggagcagctcggcctggacaccgtgagctttagcactaaaggtgccacttatattacctacgtgaatttcttgaatgagctacgagttaaattgaaacccgaaggtaacagccatggaatcccattgctgcgcaaaaaatgtgatgatcctggaaagtgtttcgttttggtagcgctttcaaatgacaatggacagttggcggaaatagctatagatgttacaagtgtttatgtggtgggctatcaagtaagaaacagatcttacttctttaaagatgctccagatgctgcttacgaaggcctcttcaaaaacacaattaaaacaagacttcattttggcggcagctatccctcgctggaaggtgagaaggcatatagagagacaacagacttgggcattgaaccattaaggattggcatcaagaaacttgatgaaaatgcgatagacaattataaaccaacggagatagctagttctctattggttgttattcaaatggtgtctgaagcagctcgattcacctttattgagaaccaaattagaaataactttcaacagagaatccgcccgacgaataatacaatcagccttgagaataaatggggtaaactctcgttccagatccggacatcaggtgcaaatggaatgttttcggaggcagttgaattggaacgtgcaaatggcaaaaaatactatgtcaccgcagttgatcaagtaaaacccaaaatagcactcttgaagttcgtcgataaagatcctaaatagtgactcgacctgcaggtctatggaacgataaatgcccatgaaaattctatttcaaggagacagtcataatgaaatacctattgcctacggcagccgctggattgttattactcgctgcccaaccagcgatggcgcatcaccatcaccatcacgatatccagatgacccagtccccgtcctccctgagtgcttctgttggtgaccgtgttaccatcacctgccgttccaccaaatccctcctgcactccaacggtatcacctacctttattggtatcaacagaaaccgggtaaagctccgaaacttctgatctaccagatgtccaacctggcttccggtgttccgtctcgtttctccagttctggttctggtaccgacttcaccctgaccatctcttctctgcagccggaagacttcgctacctactactgcgctcagaacctggaaatcccgcgtaccttcggtcagggtaccaaagttgaacttaagcgcactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgactcgag

SEQ ID NO: 10 - protein, 751 aa, Artificial Sequence
  unoptimized VB6-845-gelonin
MKYLLPTAAAGLLLLAAQPAMAEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCTRHRQPRGWEQLGLDTVSFSTKGATYITYVNFLNELRVKLKPEGNSHGIPLLRKKCDDPGKCFVLVALSNDNGQLAEIAIDVTSVYVVGYQVRNRSYFFKDAPDAAYEGLFKNTIKTRLHFGGSYPSLEGEKAYRETTDLGIEPLRIGIKKLDENAIDNYKPTEIASSLLVVIQMVSEAARFTFIENQIRNNFQQRIRPTNNTISLENKWGKLSFQIRTSGANGMFSEAVELERANGKKYYVTAVDQVKPKIALLKFVDKDPKMKYLLPTAAAGLLLLAAQPAMAHHHHHHDIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - DNA, 2773 bp, Artificial Sequence
  unoptimized VB6-845 pe
gaattcctgcaggtctatggaacgataaatgcccatgaaaattctatttcaaggagacagtcataatgaaatacctattgcctacggcagccgctggattgttattactcgctgcccaaccagcgatggcggaagtacagctggttcagtccggcccgggtcttgttcaaccgggtggttccgttcgtatctcttgcgctgcttctggttacacgttcaccaactacggcatgaactgggtcaaacaggctccgggtaaaggcctggaatggatgggctggatcaacacctacaccggtgaatccacctacgctgactccttcaaaggtcgcttcactttctccctcgacacaagtgctagtgctgcatacctccaaatcaactcgctgcgtgcagaggatacagcagtctattactgcgcccgtttcgctatcaaaggtgactactggggtcaaggcacgctgctgaccgtttcctcggcttccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgaatttggtggcgcgccggagttcccgaaaccgtccaccccgccgggttcttctggtcttgagggcggcagcctggccgcgctgaccgcgcaccaggcctgccacctgccgctggagactttcacccgtcatcgccagccgcgcggctgggaacaactggagcagtgcggctatccggtgcagcggctggtcgccctctacctggcggcgcgactgtcatggaaccaggtcgaccaggtgatccgcaacgccctggccagccccggcagcggcggcgacctgggcgaagcgatccgcgagcagccggagcaggcacgtctcgctctgaccctggccgccgccgagagcgagcgcttcgtccggcagggcaccggcaacgacgaggcaggcgctgcaagcgccgacgtggtgagcctgacctgcccggtcgccgccggtgaatgcgcgggcccggcggacagcggcgacgccctgctggagcgcaactatcccactggcgcggagttcctcggcgacggtggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggatctcgacgcgatctggcgcggtttctatatcgccggcgatccggcgctggcctacggctacgcccaggaccaggaacccgacgcgcgcggccggatccgcaacggtgccctgctgcgggtctatgtgccgcgctccagcctgccgggcttctaccgcaccggcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggtggcgacctcgacccgtccagcatccccgacaaggaacaggcgatcagcgccctgccggactacgccagccagcccggcaaaccgccgcatcaccaccatcaccataaagacgaactgtagtgactcgacctgcaggtctatggaacgataaatgcccatgaaaattctatttcaaggagacagtcataatgaaatacctattgcctacggcagccgctggattgttattactcgctgcccaaccagcgatggcgcatcaccatcaccatcacgatatccagatgacccagtccccgtcctccctgagtgcttctgttggtgaccgtgttaccatcacctgccgttccaccaaatccctcctgcactccaacggtatcacctacctttattggtatcaacagaaaccgggtaaagctccgaaacttctgatctaccagatgtccaacctggcttccggtgttccgtctcgtttctccagttctggttctggtaccgacttcaccctgaccatctcttctctgcagccggaagacttcgctacctactactgcgctcagaacctggaaatcccgcgtaccttcggtcagggtaccaaagttgaacttaagcgcactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagtgactcgag

SEQ ID NO: 12 - protein, 875 aa, Artificial Sequence [sequence omitted]

SEQ ID NO: 13 - DNA, 42 bp, Artificial Sequence; identical to SEQ ID NO: 5
  Primer

SEQ ID NO: 14 - DNA, 30 bp, Artificial Sequence
  Primer
accaggcacaggcataaaagatccgtctac

SEQ ID NO: 15 - DNA, 30 bp, Artificial Sequence
  Primer
gttgtagacggatcttttatgcctgtgcct

SEQ ID NO: 16 - protein, 9 aa, Artificial Sequence
  Linker
TRHRHKRSV

SEQ ID NO: 17 - DNA, 57 bp, Artificial Sequence
  Primer
accaggcacaggaggaaaagatccgtctacaacaccgtgtcatttaaccttggagaa

SEQ ID NO: 18 - DNA, 45 bp, Artificial Sequence
  Primer
gacggatcttttcctcctgtgcctggtacactctcccctgttgaa

SEQ ID NO: 19 - protein, 9 aa, Artificial Sequence
  Linker
TRHRRKRSV

SEQ ID NO: 20 - DNA, 57 bp, Artificial Sequence
  Primer
cagggtgtaggatttggcacccagtttttctacaacaccgtgtcatttaaccttgga

SEQ ID NO: 21 - DNA, 48 bp, Artificial Sequence
  Primer
ctgggtgccaaatcctacaccctgacactctcccctgttgaagctctt

SEQ ID NO: 22 - protein, 10 aa, Artificial Sequence
  Linker
QGVGFGTQFF

SEQ ID NO: 23 - DNA, 33 bp, Artificial Sequence
  Primer
ggatttctaggcacccagtttttctacaacacc

SEQ ID NO: 24 - DNA, 30 bp, Artificial Sequence
  Primer
gaaaaactgggtgcctagaaatcctacacc

SEQ ID NO: 25 - protein, 11 aa, Artificial Sequence
  Linker
QGVGFLGTQFF

SEQ ID NO: 26 - DNA, 57 bp, Artificial Sequence; identical to SEQ ID NO: 20
  Primer

SEQ ID NO: 27 - DNA, 60 bp, Artificial Sequence
  Primer
ctgggtgccaaatcctacaccctggagctgctcccagcctctgggctgcctgtgcctggt

SEQ ID NO: 28 - protein, 22 aa, Artificial Sequence
  Linker
TRHRQPRGWEQLQGVGFGTQFF

SEQ ID NO: 29 - DNA, 57 bp, Artificial Sequence; identical to SEQ ID NO: 20
  Primer

SEQ ID NO: 30 - DNA, 69 bp, Artificial Sequence
  Primer
ctgggtgccaaatcccataccctggacggatcttttatgcctgtgcctggtacactctcccctgttgaa

SEQ ID NO: 31 - protein, 19 aa, Artificial Sequence
  Linker
TRHRHKRSVQGVGFGTQFF

SEQ ID NO: 32 - DNA, 57 bp, Artificial Sequence; identical to SEQ ID NO: 20
  Primer

SEQ ID NO: 33 - DNA, 69 bp, Artificial Sequence
  Primer
ctgggtgccaaatcctacaccctggacggatcttttcctcctgtgcctggtacactctcccctgttgaa

SEQ ID NO: 34 - protein, 19 aa, Artificial Sequence
  Linker
TRHRRKRSVQGVGFGTQFF

SEQ ID NO: 35 - DNA, 39 bp, Artificial Sequence; identical to SEQ ID NO: 8
  Primer

SEQ ID NO: 36 - DNA, 36 bp, Artificial Sequence
  Original Furin linker unoptmized
accaggcacaggcagcccagaggctgggagcagctc

SEQ ID NO: 37 - protein, 12 aa, Artificial Sequence
  Original Furin linker unoptimized
TRHRQPRGWEQL

SEQ ID NO: 38 - DNA, 27 bp, Artificial Sequence
  F1 Furin linker unoptimized
accaggcacaggcataaaagatccgtc

SEQ ID NO: 39 - protein, 9 aa, Artificial Sequence
  F1 Furin linker unoptimized
TRHRHKRSV

SEQ ID NO: 40 - DNA, 27 bp, Artificial Sequence
  F1R Furin linker unoptimized
accaggcacaggaggaaaagatccgtc

SEQ ID NO: 41 - protein, 9 aa, Artificial Sequence
  F1R Furin linker unoptimized
TRHRRKRSV

SEQ ID NO: 42 - DNA, 30 bp, Artificial Sequence
  CB-CD linker unoptimized
cagggtgtaggatttggcacccagtttttc

SEQ ID NO: 43 - protein, 10 aa, Artificial Sequence
  CB-CD linker unoptimized
QGVGFGTQFF

SEQ ID NO: 44 - DNA, 33 bp, Artificial Sequence
  CB1-CD linker unoptimized
cagggtgtaggatttctaggcacccagtttttc

SEQ ID NO: 45 - protein, 11 aa, Artificial Sequence
  CB1-CD linker unoptimized
QGVGFLGTQFF

SEQ ID NO: 46 - DNA, 66 bp, Artificial Sequence
  F-CB-CD linker unoptimized
accaggcacaggcagcccagaggctgggagcagctccagggtgtaggatttggcacccagtttttc

SEQ ID NO: 47 - protein, 22 aa, Artificial Sequence
  F-CB-CD linker unoptimized
TRHRQPRGWEQLQGVGFGTQFF

SEQ ID NO: 48 - DNA, 57 bp, Artificial Sequence
  F1-CB-CD linker unoptimized
accaggcacaggcataaaagatccgtccagggtgtaggatttggcacccagtttttc

SEQ ID NO: 49 - protein, 19 aa, Artificial Sequence
  F1-CB-CD linker unoptimized
TRHRHKRSVQGVGFGTQFF

SEQ ID NO: 50 - DNA, 57 bp, Artificial Sequence
  F1R-CB-CD linker unoptimized
accaggcacaggaggaaaagatccgtccagggtgtaggatttggcacccagtttttc

SEQ ID NO: 51 - protein, 19 aa, Artificial Sequence
  F1R-CB-CD linker unoptimized
TRHRRKRSVQGVGFGTQFF

SEQ ID NO: 52 - DNA, 2354 bp, Artificial Sequence
  Optimized VB6-845
aaggagaattccataatgaaatatctgctgccgactgctgctgcgggtctgctgctgctggctgcgcaaccggctatggctcatcaccaccaccaccatgaggttcagctggtccagtccggtccgggtctggttcagccgggtggtagcgttcgtattagctgcgcggcgagcggttacaccttcaccaactacggtatgaactgggttaaacaggctccgggtaaaggtttggaatggatgggttggatcaacacctataccggtgagtctacctacgctgatagcttcaaaggccgtttcacctttagccttgacacttctgcgagcgcggcgtacctgcagattaactctctgcgtgctgaggacactgcggtttactactgcgctcgtttcgcgatcaaaggtgactattggggtcagggtactctgctgaccgttagcagcgctagcactaaaggtccgtccgttttcccactggctccgtcttctaaaagcacttctggtggtaccgcggctctgggttgccttgttaaagactacttccctgaaccggtcaccgttagctggaactccggtgcgttgacctctggtgttcacaccttcccagcggttctgcagtctagcggtctgtatagcctgagctctgtagttaccgttccgtcttctagcctgggtacgcagacctacatctgcaacgtgaaccacaaaccgagcaacactaaagtggataaaaaagttgaaccgaagtcttgctagtgatctagagtcgacctgcaggtctatggaacgataaatgcccatgaaaattctatttcaaggagacagtcataatgaaataccttctgccgaccgctgccgctggtctgctgctgttggctgctcaaccggctatggcagacatccagatgacccagtccccgtctagcctgagcgcaagcgttggtgaccgtgtgaccatcacctgccgtagcactaaatccctgctgcactctaacggcatcacctacctgtattggtaccaacagaaaccgggtaaagctccgaaactgctgatctaccagatgtctaacctggctagcggcgttccttctcgtttttcttctagcggtagcggtactgacttcaccctgaccattagctctctgcagcctgaagactttgcgacctactattgcgctcagaaccttgaaatcccgcgtaccttcggccagggtaccaaagttgaactgaagcgtaccgttgcggctccgtctgttttcatcttcccacctagcgatgaacagcttaaatctggtactgctagcgtagtttgcctgcttaacaacttctaccctcgtgaagctaaagttcagtggaaagttgacaacgctctgcagtctggtaactctcaggaatctgtgaccgaacaggatagcaaagatagcacctatagcctgtctagcaccctgacccttagcaaggcggactatgaaaaacacaaagtttacgcttgcgaggtgacccaccaaggtctgtcttctccggtgactaaatcctttaaccgtggcgaatgcacccgtcaccgtcagccgcgtggttgggaacagctgtataacaccgtatcttttaacctgggtgaggcgtatgaatacccgaccttcatccaggacctgcgtaatgaacttgctaaaggtacccctgtttgccagctgcctgtgaccctgcagaccatcgctgatgataaacgtttcgttctggttgacattaccaccacctccaaaaaaaccgttaaagtcgcgatcgatgtgaccgacgtttacgtggtaggttaccaggataaatgggacggtaaagatcgtgcggttttcctggacaaagttccgaccgtagcgacttctaaactgttcccaggtgtgaccaaccgtgtgaccctgaccttcgacggcagctatcagaaactggttaacgcggccaaagctgatcgtaaagctctcgaactgggtgttaacaaactggagttcagcattgaagctatccacggtaaaaccatcaacggtcaagaagcagctaaattcttcctgatcgtgatccagatggttagcgaagcagcgcgttttaaatacattgaaaccgaagtagttgatcgtggtctgtatggtagcttcaaaccgaacttcaaagttcttaacctggagaacaactggggtgacattagcgacgcgatccataaatcttccccgcaatgcaccaccattaacccggctctgcagctgatctctccgtctaacgatccgtgggtagttaacaaagtgtctcaaatcagcccggacatgggtatcctgaaatttaaatctagcaaatagtgactcgag

SEQ ID NO: 53 - protein, 750 aa, Artificial Sequence; identical to SEQ ID NO: 2
  Optimized VB6-845

SEQ ID NO: 54 - protein, 10 aa, Artificial Sequence
  Cathepsin proteolytic site
GFGSTFFAGF

SEQ ID NO: 55 - protein, 4 aa, Artificial Sequence
  Legumain proteolytic site
AANL

SEQ ID NO: 56 - DNA, 30 bp, Artificial Sequence
  Primer
tacgcttgcgaggtgacccaccaaggtctg

SEQ ID NO: 57 - DNA, 36 bp, Artificial Sequence
  Primer
gcgctcgagtcactatttgctagatttaaatttcag

SEQ ID NO: 58 - DNA, 48 bp, Artificial Sequence
  Primer
tgggaacagctggctgctaacctgtataacaccgtatcttttaacctg

SEQ ID NO: 59 - DNA, 36 bp, Artificial Sequence
  Primer
caggttagcagccagctgttcccaaccacgcggctg

SEQ ID NO: 60 - DNA, 39 bp, Artificial Sequence
  Primer
ttcgctgctaacgtctataacaccgtatcttttaacctg

SEQ ID NO: 61 - DNA, 42 bp, Artificial Sequence
  Primer
gttatacaggttagcagcgaaacccgcgaagaaggtagaacc

SEQ ID NO: 62 - DNA, 39 bp, Artificial Sequence
  Primer
gaatgcgctaacctgtataacaccgtatcttttaacctg

SEQ ID NO: 63 - DNA, 42 bp, Artificial Sequence
  Primer
gttatacaggttagcagcgcattcgccacggttaaaggattt

SEQ ID NO: 64 - DNA, 48 bp, Artificial Sequence
  Primer
ggttctaccttcttcgcgggtttctataacaccgtatcttttaacctg

SEQ ID NO: 65 - DNA, 51 bp, Artificial Sequence
  Primer
acccgcgaagaaggtagaaccgaaacccagctgttcccaaccacgcggctg

SEQ ID NO: 66 - DNA, 54 bp, Artificial Sequence
  Primer
ggtttcggttctgttcagttcgcgggtttctataacaccgtatcttttaacctg

SEQ ID NO: 67 - DNA, 54 bp, Artificial Sequence
  Primer
gaaacccgcgaactgaacagaaccgaaaccgcattcgccacggttaaaggattt

SEQ ID NO: 68 - DNA, 48 bp, Artificial Sequence
  Primer
gaatgcaccttcttcgcgggtttctataacaccgtatcttttaacctg

SEQ ID NO: 69 - DNA, 48 bp, Artificial Sequence
  Primer
gttatagaaacccgcgaagaaggtgcattcgccacggttaaaggattt

SEQ ID NO: 70 - protein, 16 aa, Artificial Sequence
  Linker
TRHRQPRGWEQLAANL

SEQ ID NO: 71 - protein, 26 aa, Artificial Sequence
  Linker
TRHRQPRGWEQLGFGSTFFAGFAANL

SEQ ID NO: 72 - protein, 4 aa, Artificial Sequence
  Linker
AANL

SEQ ID NO: 73 - protein, 22 aa, Artificial Sequence
  Linker
TRHRQPRGWEQLGFGSTFFAGF

SEQ ID NO: 74 - protein, 10 aa, Artificial Sequence
  Linker
GFGSVQFAGF

SEQ ID NO: 75 - protein, 6 aa, Artificial Sequence
  Linker
TFFAGF

SEQ ID NO: 76 - DNA, 36 bp, Artificial Sequence
  Furin linker optimized
acccgtcaccgtcagccgcgtggttgggaacagctg

SEQ ID NO: 77 - protein, 12 aa, Artificial Sequence
  Furin linker optimized
TRHRQPRGWEQL

SEQ ID NO: 78 - DNA, 48 bp, Artificial Sequence
  F-Leg Linker
acccgtcaccgtcagccgcgtggttgggaacagctggctgctaacctg

SEQ ID NO: 79 - protein, 16 aa, Artificial Sequence
  F-Leg Linker
TRHRQPRGWEQLAANL

SEQ ID NO: 80 - DNA, 12 bp, Artificial Sequence
  Leg linker
gctgctaacctg

SEQ ID NO: 81 - protein, 4 aa, Artificial Sequence
  Leg linker
AANL

SEQ ID NO: 82 - DNA, 30 bp, Artificial Sequence
  CB linker
ggtttcggttctgttcagttcgcgggtttc

SEQ ID NO: 83 - protein, 10 aa, Artificial Sequence
  CB linker
GFGSVQFAGF

SEQ ID NO: 84 - DNA, 18 bp, Artificial Sequence
  CD linker
accttcttcgcgggtttc

SEQ ID NO: 85 - protein, 6 aa, Artificial Sequence
  CD linker
TFFAGF

SEQ ID NO: 86 - DNA, 66 bp, Artificial Sequence
  F-CB-CD linker
acccgtcaccgtcagccgcgtggttgggaacagctgggtttcggttctaccttcttcgcgggtttc

SEQ ID NO: 87 - protein, 22 aa, Artificial Sequence
  F-CB-CD linker
TRHRQPRGWEQLGFGSTFFAGF

SEQ ID NO: 88 - DNA, 78 bp, Artificial Sequence
  F-CB-CD-Leg linker
acccgtcaccgtcagccgcgtggttgggaacagctgggtttcggttctaccttcttcgcgggtttcgctgctaacctg

SEQ ID NO: 89 - protein, 26 aa, Artificial Sequence
  F-CB-CD-Leg linker
TRHRQPRGWEQLGFGSTFFAGFAANL

SEQ ID NO: 90 - protein, 250 aa, Artificial Sequence
  De-bouganin
YNTVSFNLGEAYEYPTFIQDLRNELAKGTPVCQLPVTLQTIADDKRFVLVDITTTSKKTVKVAIDVTDVYVVGYQDKWDGKDRAVFLDKVPTVATSKLFPGVTNRVTLTFDGSYQKLVNAAKADRKALELGVNKLEFSIEAIHGKTINGQEAAKFFLIVIQMVSEAARFKYIETEVVDRGLYGSFKPNFKVLNLENNWGDISDAIHKSSPQCTTINPALQLISPSNDPWVVNKVSQISPDMGILKFKSSK